(19) United States
(12) Patent Application Publication
(10) Pub. No.: US 2005/0002953 A1
(43) Pub. Date: Jan. 6, 2005

(54) SARS-coronavirus virus-like particles and methods of use

(76) Inventor: Jens Herold, Puchheim (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/839,729

(22) Filed: May 4, 2004

Related U.S. Application Data
(60) Provisional application No. 60/468,703, filed on May 6, 2003.

(51) Int. Cl. A61K 39/12; A61K 39/215; C12Q 1/70; C12N 7/00

(52) U.S. Cl. 424/186.1; 424/221.1; 435/5; 435/252.34; 435/235.1

(57) ABSTRACT

The present disclosure describes a system for making SARS-CoV-virus-like particles (SARS-CoV-VLPs) comprising one or more recombinant vectors which express the SARS-CoV E-protein, the SARS-CoV M-protein, and the SARS-CoV S-protein. Additionally, the present disclosure describes methods of inducing an immune response in a subject comprising administering to the subject a nucleic acid encoding the SARS-CoV E-protein, the SARS-CoV M-protein, and the SARS-CoV S-protein. Methods of inducing an immune response in a subject comprising administering to the subject SARS-CoV-VLPs are also disclosed.

### RELATED APPLICATIONS

This application is a nonprovisional application which claims priority under 35 U.S.C. § 119(e) to U.S. Provisional Patent Application Number 60/468,703, entitled SARS-CORONAVIRUS-LIKE PARTICLES AND METHODS OF USE, filed May 6, 2003, the disclosure of which is incorporated herein by reference in its entirety.

### FIELD OF THE INVENTION

The present invention relates to the fields of biotechnology and medicine. In particular, the present invention relates SARS-coronavirus-like particles, methods of making such particles and methods of using these particles to elicit an immune response.

### BACKGROUND

Outbreaks of SARS coronavirus (SARS-CoV) have recently caused a large number of deaths around the world, especially in China, Canada and Vietnam. Fatalities are typically caused by progressive respiratory failure which occurs in up to about ten percent of all SARS cases. Currently, there exists no cure for SARS and no means by which to reduce the rate of mortality associated with this disease. Additionally, there exists no means by which to reliably prevent SARS infection nor is there any means by which to reduce the symptoms associated with such infection. In view of the considerable impact of SARS and the lack of a suitable treatment, compositions and/or methods for ameliorating the effects of this disease are much needed.

### SUMMARY OF THE INVENTION

Some embodiments of the present invention relate to systems, such as recombinant plasmids, viruses and prokaryotes, that express the SARS-CoV membrane-associated proteins M, E and S in cells, such as human cells, both in vitro and in vivo. In some embodiments, the SARS-CoV M, E and S proteins spontaneously form SARS-CoV-virus-like particles (SARS-CoV-VLPs). In such embodiments, the SARS-CoV-VLPs can be secreted by the cell.

According to other embodiments of the present invention, intracellular expression of the SARS-CoV M, E and S proteins and their association to form virus-like particles, which present the viral proteins in their “natural” context, causes the induction of an immune response. As such, some embodiments of the present invention relate to methods of producing an immune response in animals, such as humans and other mammals, by identifying a subject at risk for developing SARS and administering to the subject one or more genetic constructs capable of expressing the SARS-CoV M, E and/or S polypeptides. In some embodiments, the one or more genetic constructs express the SARS-CoV M, E and S polypeptides which spontaneously form SARS-CoV-VLPs. In a preferred embodiment, both, a strong antibody response as well a strong cytotoxic T lymphocyte (CTL) response are induced. In other embodiments, only one of either an antibody response or a CTL response is induced.

Certain embodiments of the present invention relate to the SARS-CoV-VLPs and methods of producing these particles. Other embodiments relate to the administration of SARS-CoV-VLPs to an animal, such as a human or other mammal, so as to generate an immune response in the animal.

In some embodiments of the present invention, VLPs that are produced contain an E protein which is selected from the group consisting of SEQ ID NOs: 2-5 or portions thereof. In other embodiments, VLPs that are produced contain an M protein which is selected from the group consisting of SEQ ID NOs: 6-9 or portions thereof. In still other embodiments, VLPs that are produced contain an S protein which is selected from the group consisting of SEQ ID NOs: 10-13 or portion thereof. Portions of the E protein can include at least about 6 consecutive amino acids, at least about 7 consecutive amino acids, at least about 8 consecutive amino acids, at least about 9 consecutive amino acids, at least about 10 consecutive amino acids, at least about 11 consecutive amino acids, at least about 12 consecutive amino acids, at least about 13 consecutive amino acids, at least about 14 consecutive amino acids, at least about 15 consecutive amino acids, at least about 16 consecutive amino acids, at least about 17 consecutive amino acids, at least about 18 consecutive amino acids, at least about 19 consecutive amino acids, at least about 20 consecutive amino acids, at least about 25 consecutive amino acids, at least about 30 consecutive amino acids, at least about 40 consecutive amino acids, at least about 50 consecutive amino acids, at least about 60 consecutive amino acids, at least about 70 consecutive amino acids or greater than 70 amino acids. Portions of the M protein can include at least about 6 consecutive amino acids, at least about 7 consecutive amino acids, at least about 8 consecutive amino acids, at least about 9 consecutive amino acids, at least about 10 consecutive amino acids, at least about 11 consecutive amino acids, at least about 12 consecutive amino acids, at least about 13 consecutive amino acids, at least about 14 consecutive amino acids, at least about 15 consecutive amino acids, at least about 16 consecutive amino acids, at least about 17 consecutive amino acids, at least about 18 consecutive amino acids, at least about 19 consecutive amino acids, at least about 20 consecutive amino acids, at least about 25 consecutive amino acids, at least about 30 consecutive amino acids, at least about 40 consecutive amino acids, at least about 50 consecutive amino acids, at least about 60 consecutive amino acids, at least about 70 consecutive amino acids, at least about 80 consecutive amino acids, at least about 90 consecutive amino acids, at least about 100 consecutive amino acids, at least about 120 consecutive amino acids, at least about 140 consecutive amino acids, at least about 160 consecutive amino acids, at least about 180 consecutive amino acids, at least about 200 consecutive amino acids, or greater than 200 consecutive amino acids. Portions of the S protein can include at least about 6 consecutive amino acids, at least about 7 consecutive amino acids, at least about 8 consecutive amino acids, at least about 9 consecutive amino acids, at least about 10 consecutive amino acids, at least about 11 consecutive amino acids, at least about 12 consecutive amino acids, at least about 13 consecutive amino acids, at least about 14 consecutive amino acids, at least about 15 consecutive amino acids, at least about 16 consecutive amino acids, at least about 17 consecutive amino acids, at least about 18 consecutive amino acids, at least about 19 consecutive amino acids, at least about 20 consecutive amino acids, at least about 25 consecutive amino acids, at least about 30 consecutive amino acids, at least about 40 consecutive amino acids, at least about 50 consecutive amino acids, at least about 60 consecutive amino acids, at least about 70 consecutive amino acids, at least about 80 consecutive amino acids, at least about 90 consecutive amino acids, at least about 100 consecutive amino acids, at least about 120 consecutive amino acids, at least about 140 consecutive amino acids, at least about 160 consecutive amino acids, at least about 180 consecutive amino acids, at least about 200 consecutive amino acids, at least about 250 consecutive amino acids, at least about 300 consecutive amino acids, at least about 350 consecutive amino acids, at least about 400 consecutive amino acids, at least about 450 consecutive amino acids, at least about 500 consecutive amino acids, at least about 550 consecutive amino acids, at least about 600 consecutive amino acids, at least about 650 consecutive amino acids, at least about 700 consecutive amino acids, at least about 750 consecutive amino acids, at least about 800 consecutive amino acids, at least about 850 consecutive amino acids, at least about 900 consecutive amino acids, at least about 950 consecutive amino acids, at least about 1000 consecutive amino acids, at least about 1050 consecutive amino acids, at least about 1100 consecutive amino acids, at least about 1150 consecutive amino acids, at least about 1200 consecutive amino acids, at least about 1250 consecutive amino acids or greater than 1250 consecutive amino acids.

The systems and methods described herein are useful to reduce the symptoms of SARS-CoV infections.

Additional aspects of the present invention are provided in the following numbered paragraphs:
1. A system for making SARS-CoV virus-like particles (SARS-CoV-VLPs) comprising one or more recombinant vectors which express the SARS-CoV E-protein, the SARS-CoV M-protein and the SARS-CoV S-protein.
2. The system of claim 1, wherein said SARS-CoV E-protein, said SARS-CoV M-protein and said SARS-CoV S-protein are expressed from a single recombinant vector.
3. The system of claim 1, wherein said SARS-CoV E-protein, said SARS-CoV M-protein, and said SARS-CoV S-protein are expressed from a plurality of recombinant vectors.
4. The system of claim 1, wherein said one or more recombinant vectors comprise a plasmid.
5. The system of claim 1, wherein said one or more recombinant vectors comprise a recombinant virus.
6. The system of claim 5, wherein said recombinant virus is a measles virus.
7. A cell which has been engineered to express the SARS-CoV E-protein, the SARS-CoV M-protein, and the SARS-CoV S-protein.
8. The cell of claim 7, wherein said cell is live.
9. The cell of claim 8, wherein said cell is a bacterial cell.
10. The cell of claim 9, wherein said cell is a bacterial cell whose pathogenicity has been attenuated.
11. The cell of claim 10, wherein said cell is a Salmonella cell.
12. A method of inducing an immune response comprising administering to a subject one or more recombinant vectors which express the SARS-CoV E-protein, the SARS-CoV M-protein and the SARS-CoV S-protein.
13. The method of claim 12, wherein said SARS-CoV E-protein, the SARS-CoV M-protein and the SARS-CoV S-protein are expressed from a single recombinant vector.
14. The method of claim 12, wherein said SARS-CoV E-protein, the SARS-CoV M-protein and the SARS-CoV S-protein are expressed from a plurality of recombinant vectors.
15. The method of claim 12, wherein said one or more recombinant vectors comprise a plasmid.
16. The method of claim 12, wherein said one or more recombinant vectors comprise a virus.
17. The method of claim 12, wherein said one or more recombinant vectors comprise a prokaryotic vector.
18. The method of claim 12, wherein said subject is a human.
19. The method of claim 18, wherein said immune response is a cellular immune response.
20. The method of claim 18, wherein said immune response is a humoral immune response.
21. The method of claim 18, wherein said immune response is both a humoral and a cellular immune response.
22. A method of inducing an immune response in a subject comprising administering SARS-CoV-VLPs to said subject.
23. A method of inducing an immune response in a subject comprising administering a nucleic acid encoding the SARS-CoV E-protein, the SARS-CoV M-protein, and the SARS-CoV S-protein to said subject.
24. A SARS-CoV-VLP.
25. An isolated SARS-CoV-VLP of claim 24.

### BRIEF DESCRIPTION OF THE DRAWINGS

FIG. 1 depicts a schematic representation of a coronavirion. The genomic RNA is encapsidated by the nucleocapsid protein N. The membrane protein M, the spike protein S and the E protein are embedded in the lipid bilayer. Several coronaviruses also contain a fourth envelope protein, the hemagglutinin esterase protein HE (not shown).

FIG. 2 shows the genomic organization of coronaviruses. The main open reading frames encoded by the genomic RNA of MHV, HCV 229E, IBV and SARS-CoV are shown. The genes encoding for the integral envelope proteins S, M, and E, are shown as closed boxes.

FIG. 3 depicts coronavirus gene expression. A coterminal nested set of mRNAs is expressed. Only the unique region of an mRNA that is not contained in the next smaller mRNA is translationally active. The genes encoding for the integral envelope proteins S, M, and E, are shown as closed boxes.

FIG. 4A depicts an alignment of E protein amino acid sequences from four SARS-CoV isolates: Tor2, Urbani, HKU-39849 and CUHK-W1.

FIG. 4B depicts an alignment of the M protein amino acid sequences from four SARS-CoV isolates: Tor2, Urbani, HKU-39849 and CUHK-W1. Highlighting indicates residues that are not identical between each of the four isolates.

FIG. 4C depicts an alignment of the S protein amino acid sequences from four SARS-CoV isolates: Tor2, Urbani, HKU-39849 and CUHK-W1. Highlighting indicates resides that are not identical between each of the four isolates.

FIG. 5 depicts a transfected tissue culture cell producing SARS-CoV proteins upon transfection of the plasmids carrying the relevant genes under control of a eukaryotic promoter

FIG. 6 depicts tissue culture cells producing SARS-CoV proteins upon infection with recombinant viruses carrying the relevant genes under control of a eukaryotic promoter

FIG. 7 depicts tissue culture cells producing SARS-CoV proteins upon addition of a prokaryotic vector carrying the SARS-CoV S—, M- and E-genes under control of a eukaryotic promoter

FIG. 8 depicts tissue culture cells producing SARS-CoV-like particles upon expression of the SARS-CoV S—, M- and E-genes. The particles are released into the tissue culture supernatant.

FIG. 9 schematically illustrates the results of density gradient centrifugation of intact virus particle not treated with Triton-X100 (no) and the migration of solubilized proteins (Triton-X100) in a sucrose gradient.

### DETAILED DESCRIPTION

Some aspects of the present invention provide delivery vectors which express the products of the SARS coronavirus (SARS-CoV) M, E and S genes. The delivery vectors can be any type of vector compatible with this purpose. For example, the vectors can be plasmid vectors, viral vectors or prokaryotic vectors. The vectors produce SARS-CoV-virus-like particles (SARS-CoV-VLPs) in vivo and can comprise any of the following:
a. one vector carrying all three genes (M, E, and S), or
b. two vectors, one carrying a combination of any two of the foregoing genes, the other carrying one of the foregoing genes, or
c. three vectors, each carrying one of the foregoing genes.

SARS-CoV-like particles are useful for stimulating an immune response in an animal without producing illness or SARS-related symptoms.

An effective host defense against coronavirus associated infectious diseases may be obtained by stimulating the cellular and/or humoral immune system. In one embodiment, the system disclosed herein will stimulate a T-cell response because of the intracellular expression of viral antigens and the production of highly efficient antibodies via the release of native envelope proteins as components of virus-like particles (VLPs). In another embodiment, the antigens will be produced and presented at the mucosal sites, such as the lung.

The VLPs can be used to induce an immune response in a desired host, such as a human. While a fully protective immune response is desirable, it will be appreciated that an immune response which is not fully protective is also beneficial. Accordingly, the present invention contemplates induction of a fully protective immune response as well as an immune response which is not fully protective.

In some embodiments of the present invention, SARS-CoV-VLPs are produced using genes or polypeptides of the SARS coronavirus. For example, one embodiment of the present invention contemplates the use of vectors for the expression of the SARS-CoV M, E and S genes or portions thereof which are sufficient to produce VLPs. Examples of vectors used for such expression are any vectors suitable for the efficient expression of the encoded proteins in a suitable cell type. Such vectors can include, but are not limited to, plasmid vectors, viral vectors and prokaryotic vectors. By “prokaryotic vector” is meant a microorganism comprising one or more plasmids having one or more genes which encode one or more SARS-CoV-related particles. Examples of suitable cell types for use which such vectors are those from humans and other mammals.

In some embodiments of the present invention, a suitable delivery system for the transfer of DNA into the cells, such as human cells, is used. For example, in one embodiment, naked DNA can be delivered intradermally. In another embodiment, the delivery system can deliver all three genes into lung cells in order to induce mucosal immunity at the site of infection. The genes may be delivered using a single plasmid, multiple plasmids or other systems that are able to transfer several genes at once, for example, prokaryotic or viral gene delivery systems.

In some embodiments of the present invention, the SARS-CoV genes M, E and S are expressed from the vectors supplied to tissue cultures. In such embodiments, the SARS-CoV M, E and S proteins are incorporated into VLPs that are released from the tissue culture cells. In certain embodiments, the SARS-CoV-VLPs expressed from the vectors disclosed in this invention induce a humoral and/or cellular immune response when expressed in vivo.

In other embodiments of the present invention, the SARS-CoV-VLPs produced from cell cultures are isolated, formulated as an immunogen and administered to an subject at risk for becoming infected with SARS thereby inducing a humoral and/or cellular immune response in the subject.

Coronavirus Classification

Coronaviruses are members of the nidovirales, an order that was established at the 10^{th} International Congress of Virology (Jerusalem, 1996). The order consists of the families coronaviridae and arteriviridae, positive-strand RNA viruses that were grouped into the same order because of their similarities in genome organization and their similar replication strategy. Coronaviruses were named after their characteristic appearance in the electron microscope resembling the corona solis, caused by the large spike proteins projecting from the virion surface.

Coronaviruses infect a variety of mammals including man causing primarily respiratory or enteric infections. Examples of coronaviruses that cluster in at least three distinct antigenic groups as well as their respective hosts are given in Table 1. Recent studies suggest that the SARS coronavirus might be a member of the type II coronavirus group.

TABLE 1
Coronaviruses and Their Hosts

| Group | Host | Name | Acronym | Disease |
|---|---|---|---|---|
| I | Mouse | Mouse hepatitis virus | MHV | hepatitis/ |
| | | | | encephalitis/ |
| | | | | enteric |
| | Cattle | Bovine coronavirus | BCV | enteritis |
| | Man | Human coronavirus OC43 | HCoV | enteritis |
| | | | OC43 | |
| | Pig | Porcine heamagglutinating | HEV | respiratory |
| | | encephalomyelitis virus | | infection |
| | Rat | Rat coronavirus | RCV | respiratory |
| | | | | infection |
| | Turkey | Turkey coronavirus | TCV | respiratory |
| | | | | infection |
| II | Dog | Canine coronavirus | CCV | enteritis |
| | Cat | Feline infectious peritonitis | FIPV | respiratory |
| | | virus | | infection |
| | Cat | Feline enteric coronavirus | FECV | enteritis |
| | Man | Human coronavirus 229E | HCoV | respiratory |
| | | | 229E | infection |
| | Pig | Porcine epidemic diarrhea | PEDV | enteritis |
| | | virus | | |
| | Pig | Porcine transmissible | TGEV | enteritis |
| | | gastroenteritis virus | | |
| | Turkey | Turkey coronavirus | TCV | respiratory |
| | | | | infection |
| III | Chicken | Avian infectious bronchitis | IBV | respiratory |
| | | virus | | infection |
| Unas- | Man | Severe acute respiratory | SARS | respiratory |
| signed | | syndrome coronavirus | CoV | infection |

Coronavirus Structure

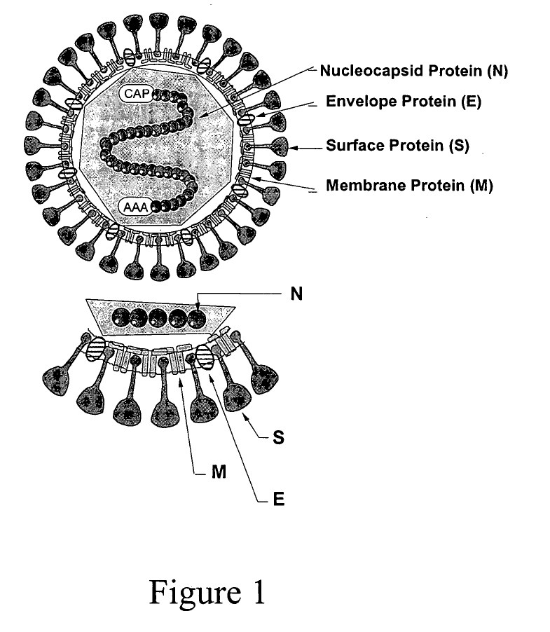

Coronaviruses are enveloped viruses. The virions are 80-200 nm pleomorphic particles and the lipid bilayer of host cell origin surrounds the genomic RNA that is encapsidated by the nucleocapsid protein. Evidence from early studies suggests that the packaging form of the coronavirus nucleocapsid is helical (MacNaughton et al, 1978). Newer data, however, appears to indicate that, at a higher order, the nucleocapsid is packaged in an icosahedral form in the virion (Risco et al, 1996). A schematic representation of a coronavirion is depicted in FIG. 1.

In addition to the nucleocapsid structure, coronaviruses contain various other structural polypeptides. For example, coronaviruses contain the triple membrane spanning M protein (20-25 kD), which is the most abundant envelope protein. Another structural protein is the spike protein, S (180 kD), which forms peplomers on the virion surface. S binds to the coronavirus receptor and induces both cell-to-cell fusion and virus-to-cell fusion as well as neutralizing antibodies. Recently, the small envelope protein, E, was discovered to be part of the virion (Liu and Inglis, 1991). The E protein appears to be necessary for virus assembly (Venemma et al., 1996). Each of the above-mentioned structural features are depicted in FIG. 1.

Coronavirus Genome Organization

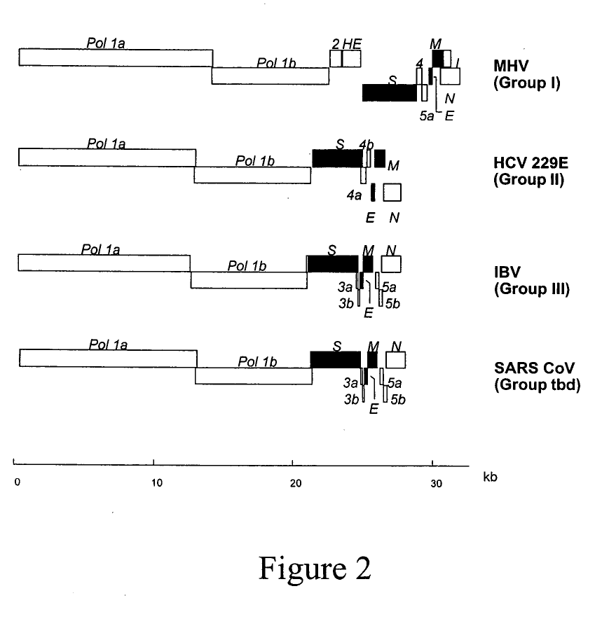

The genomic RNA of coronaviruses encompasses 27-32 kB. The 5′-two thirds of the genome encodes the replicase gene in two large overlapping open reading frames. The structural proteins S, M, E and N are encoded at the 3′-end of the genome (see FIG. 2). Additionally, there are a couple of small open reading frames (ORFs) interspersed between the structural genes. It is not always clear, however, if and how these ORFs are expressed nor is the role of these open reading frames always clear. There has been some speculation that these small ORFs may play some role in viral pathogenesis.

Coronavirus Replication and Gene Expression

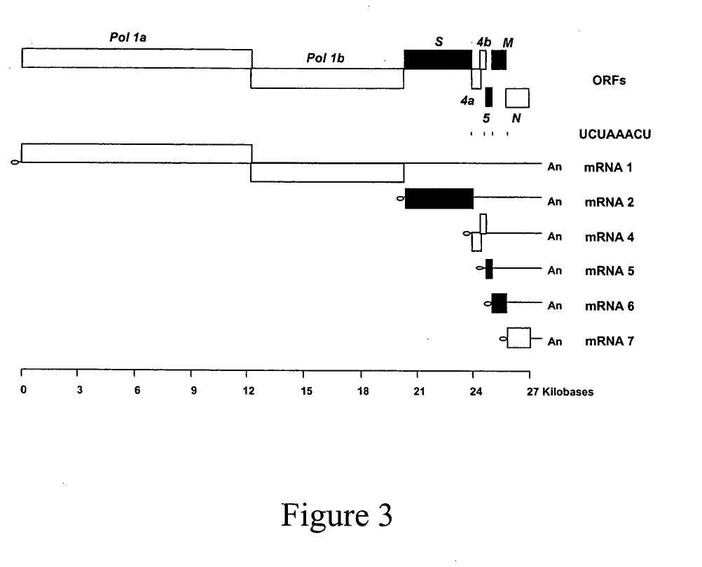

Upon infection, the coronavirus RNA is translated to produce an RNA-dependent RNA polymerase encoded by the overlapping open reading frames 1a and 1b. The latter ORF is only expressed after a (−1) ribosomal frameshifting event which occurs at a frequency of up to about 30%. Since the coronavirus genome is of positive polarity, negative strand RNA synthesis occurs next in the replication cycle. The negative stranded RNA in turn serves as a template for new positive stranded genomic RNA. All genes other than the replicase are translated from a nested set of 3′-coterminal mRNAs which contain a unique region at the 5-end that is not included in the next smaller mRNA and which include one or more ORFs. In general, the coronavirus genome includes a transcription associated sequence (TAS) element which precedes each open reading frame. FIG. 3 shows typical coronavirus genome organization using HCV 229E as an example. For HCV 229E the TAS is UCUAAACU (SEQ ID NO: 1).

The Membrane-Associated Coronaviral Structural Proteins M, E and S

The M Protein

The M protein is the most abundant membrane protein in the coronavirus virion. This protein spans the viral membrane three times such that the N-terminus is situated outside the virion and the C-terminus is inside (Armstrong et al. 1984, Rottier at al., 1986). M has a long cytoplasmic tail of approximately 100 amino acids that is probably embedded in the membrane. The M proteins of most coronaviruses are either N— or O-glycosylated.

The M protein is essential for virion formation (Holmes et al., 1981, Rottier at al., 1981). For example, interaction between M and S is important for insertion of the peplomers into the virions (Opstelten et al., 1994, 1995). Additionally, interaction between M and N is likely to be necessary for incorporation of the core into the budding virion (Sturman et al., 1980).

The Small Membrane Protein E

The small membrane protein E, which is approximately 10 kD, was not recognized as a structural protein until the early 1990s (Liu and Inglis, 1991). E is a highly hydrophobic membrane protein but contains many charged residues in the C-terminus. In TGEV, the C-terminus of this protein has been shown to be located outside of the membrane (Godet et al., 1992). E appears to be neither glycosylated nor phosphorylated. Although it is clear that the E protein is an important protein for virion assembly (Vennema et al., 1996), its definitive function in this process remains to be elucidated.

The S Protein

The spike proteins of coronaviruses are type I glycoproteins of 1100 to 1450 amino acids. S proteins of some coronaviruses are proteolytically cleaved into two subunits, S1 and S2. The role for that cleavage, however, remains to be elucidated. A comparison of the spike protein sequences of different coronaviruses shows that the S2 subunit is much more conserved than the S1 subunit (Cavanagh, 1995). A signal sequence is predicted at the N-terminus of the protein that is predicted to be cleaved upon membrane translocation in the ER. Up to 35 potential N-glycosylation sites exist in the ectodomain of the spike protein but no obvious fusion peptide is detectable. A transmembrane anchor has been identified close to the carboxy terminus of the spike protein.

Using MHV A59 as an example, the maturation and transport of coronavirus spike proteins is described. First, the spike protein is synthesized as a 120 kD protein that is co-translationally glycosylated (Niemann and Klenk, 1981). Additionally, some of the 42 cysteine residues in the ectodomain form intrachain disulfide bridges (Luytyes et al., 1987). The S monomers oligomerize slowly in the ER which probably involves certain heptad repeat regions (Venemma et al., 1990; Delmas and Laude et al., 1990). Prior to oligomerization, some of the S proteins interact with the monomeric M proteins in the ER, which is a prerequisite for later incorporation into the virions (Opstelten et al., 1993, 1994). Upon proper folding, the spike proteins migrate to the intermediate compartment where they become palmitylated. It is the intermediate compartment which is the budding site (Tooze et al., 1987). Since M and E are alone sufficient for virus envelope assembly (Venemma et al, 1996), only S proteins that have bound to M are inserted into the budding virus (Opstelten et al., 1994, 1995). Assembled virions travel to the surface of infected cells using the vesicles of the constitutive pathway (Tooze et al., 1987). The contents of the vesicles are released when the vesicles fuse with the membrane.

The S-protein is the major determinant for host cell tropism. In addition to being responsible for the fusion between virus envelope and cell membrane, S is the viral protein that is recognized by the viral receptor, e.g. hCD13 (Aminopeptidase N) in the case of HCoV 229E.

Epidemiology and Pathogenesis

The epidemiology and pathogenesis of coronavirus is described below using human respiratory and pig enteric coronaviruses as examples. Human coronaviruses have first been described as the cause of acute respiratory diseases in the early 1960s in the US and the UK. In 1965, Tyrell et al. isolated human coronavirus strain B814 from a nasal lavage of a boy having a cold. A year later, in Chicago, strain 229E was adapted to growth in tissue culture (Hamre and Prochnow, 1966). In 1967, Macintosh et al. isolated a series of strains that could only replicate in organ culture. Of these strains, HCV OC43 and HCV 229E, are today recognized as the prototypes of two serologically distinct groups of human coronaviruses.

In a series of clinical studies on the epidemiology of coronaviruses, it has been shown that more than 20% of all acute respiratory diseases are caused by comonaviruses (Cavallaro and Monto, 1970; Macnaughton at al., 1983). Together with Rhino-, Adeno- and Paramyxoviruses they are the most common cause for this type of disease. Usually human coronaviruses infect the epithelial cells of the upper respiratory tract. The clinical symptoms associated with the infection are headache, fever, coughing and sneezing. It has been reported, however, that coronaviruses can also cause respiratory diseases with more severe symptoms (Matsumoto and Kawana, 1992). The main route of transmission is by the aerosols of respiratory secretions or by mechanical transmission. Furthermore, it has been shown that members of Group 1 human coronaviruses are also associated with gastrointestinal diseases (Zhang et al., 1994). Re-infections occur throughout life, indicating that it may be beneficial to frequently vaccinate against coronavirus infection.

The infection of pigs by TGEV and PEDV can cause severe problems in the meat production industry. An immunological study in Switzerland dated from 1987 revealed that 50% of animals with acute enteric problems were seropositive for PEDV. Furthermore, the percentage of seropositive animals per herd ranged from 17-100% (Hofmann and Wyler, 1987). Infectious virus is mainly transmitted through the feces of infected animals. The pathogenic role of these coronaviruses is largely caused by diarrhea in weaned pigs, feeder pigs and fattening swine. The clinical signs of infection are watery diarrhea sometimes preceded by vomiting and depression. The infection and the destruction of epithelium results in dehydration of the infected animals. The infection of young animals can lead to their death. Very similar pathogenic mechanisms of PED and TGE cause the same immunological situation. Protection against virus infection is based on intestinal mucosal immunity, which is limited to a short period after infection. Lactogenic immunity, but not circulating antibodies are protective for suckling piglets. There are no vaccines that are currently available that would protect pigs from infection.

Immune Responses to Coronavirus Infection

Induction of the Humoral Immune Response

Mice infected with MHV produce antibodies primarily against S, but also against M, E and N. Some monoclonal antibodies directed against S neutralize the virus in vitro although viral mutants which escape the neutralizing antibodies develop in tissue culture (Grosse et al. 1993). Kolb et al. demonstrated that antibodies in the milk of recombinant animals as well as those occurring naturally through acquired lactogenic immunity confer protection to the offspring. In mammals, passive immunity is provided by neutralizing antibodies passed to the offspring via the placenta or the milk as immunoglobulin G and secreted immunoglobulin A. Mice have been generated that carry transgenes which encode the light and heavy chains of an antibody that is able to neutralize the neurotropic JHM strain of murine hepatitis virus (MHV-JHM). MHV-JHM causes acute encephalitis and acute and chronic demyelination in susceptible strains of mice and rats. In vitro analysis of milk derived from different transgenic lines revealed a linear correlation between antibody expression and virus-neutralizing activity, indicating that the recombinant antibody is the major determinant of MHV-JHM neutralization in murine milk.

In previous experiments, offspring of transgenic and control mice were challenged with a lethal dose of MHV-JHM. Litters suckling nontransgenic dams succumbed to fatal encephalitis, whereas litters suckling transgenic dams were fully protected against challenge, irrespective of whether they were transgenic. Such experiments demonstrate that a single neutralizing antibody expressed in the milk of transgenic mice is sufficient to completely protect suckling offspring against MHV-JHM-induced encephalitis.

Induction of the Cellular Immune Response

In the past, there have been several reports on the importance of the cellular immune response to clear a coronavirus infection. Most recently, Seo et al. reported that Infectious Bronchitis Virus (IBV) infection and associated illness may be dramatically modified by passive transfer of immune T lymphocytes. In particular, lymphocytes collected 10 days post infection were transferred to naive chicks before challenge with virus. As determined by respiratory illness and viral load, transfer of syngenic immune T lymphocytes protected chicks from challenge infection, whereas no protection was observed in the chicks receiving the MHC compatible lymphocytes from uninfected chicks. Nearly complete elimination of viral infection and illness was observed in chicks receiving cells enriched in alphabeta lymphocytes. In contrast, removal of gammadelta T lymphocytes had only a small effect on their potential to protect chicks. The adoptive transfer of enriched CD8(+) or CD4(+) T lymphocytes indicated that protection was also a function primarily of CD8-bearing cells. These results indicated that alphabeta T lymphocytes bearing CD8(+) antigens are important in protecting chicks from IBV infection.

Taking these data together, it may be beneficial to stimulate both arms of the immune system in order to protect the host from infection and to clear the virus upon infection. However, it will be appreciated that beneficial results may also be obtained by stimulating only one arm of the immune system. Embodiments of the present invention specifically contemplate approaches for the stimulation of one and/or both arms of the immune system.

Current Vaccine Approaches in Veterinary Medicine

Subunit Vaccines

The gene encoding the fusogenic spike protein of the coronavirus causing feline infectious peritonitis has been recombined into the genome of vaccinia virus (Vennema et al., 1990). This recombinant vector induced spike-protein-specific, in vitro neutralizing antibodies in mice. When kittens were immunized with the recombinant virus, however, only low titers of neutralizing antibodies were obtained. As such, no protection was observed.

In a second report by Venemma et al., the effect of similar immunizations with the FIPV membrane (M) and nucleocapsid (N) proteins were evaluated. Vaccinia virus recombinants expressing the cloned genes induced antibodies in immunized kittens. Immunization with the N protein recombinant had no apparent effect on the outcome of challenge. However, three of eight kittens immunized with the M protein recombinant survived the challenge, as compared to one of eight kittens of the control group. Because of the small sample size, however, these numbers are not statistically significant.

Attenuated Viruses

In 1989, Christianson et al. developed a temperature-sensitive (ts) FIPV strain that replicates at 31° C., but not at 39° C. The strain was generated after 99 serial passages in tissue culture and simultaneous UV-irradiation. This ts strain was marketed as an FIPV vaccine by Pfizer in 1991 under the brand name of Primucell. The vaccine is delivered by the intranasal route and since the virus is temperature-sensitive it only replicates weakly in the “cold” upper respiratory tract. This attenuated strain is probably the most effective coronavirus vaccine; however, there have been a number of reports where this vaccine failed to decrease the FIPV infection incidence in the vaccinated group compared to a control group.

Inactivated Viruses

In 1984, Cavanagh et al. reported inoculating chickens with sucrose gradient purified IBV proteins and then challenging the inoculated birds with IBV. Although the S-protein caused antibody production, it was ineffective to impart IBV protection/resistance to the inoculated chickens, as evidenced by their susceptibility to the characteristic IBV respiratory infection.

Rotavec Corona is a marketed combination vaccine containing inactivated bovine rotavirus, bovine coronavirus and E. coli F5 (K99). According to the label, this product is not used to prevent infection, but rather, it is used to reduce virus shedding. Since the product cannot be used to prevent infection, its efficacy as a vaccine is not very high.

In a recent field study, Takamura et al. (2002) used extracts from bovine coronavirus infected cells to inoculate Holstein dairy cows intramuscularly. Not surprisingly, the vaccine was (i) safe and (ii) able to induce an antibody response. However, protection data were not included in the study report.

In summary, it appears that inactivated virus preparations do not seem to be a promising way for the development of an effective preventive measure against coronavirus infection. The present invention provides a more beneficial approach to induce an immune response against the SARS virus.

Severe Acute Respiratory Syndrome (SARS)

Several hundred cases of severe, atypical pneumonia of unknown etiology were reported in Guangdong Province of the People's Republic of China beginning in late 2002. After similar cases were detected in patients in Hong Kong, Vietnam, and Canada during February and March 2003, the World Health Organization (WHO) issued a global alert for the illness, designated “severe acute respiratory syndrome” (SARS). By late April 2003, over 4300 SARS cases and 250 SARS-related deaths were reported to WHO from over 25 countries around the world.

The severity of the effects of SARS is variable. The incubation period for the disease is usually from 2 to 7 days. Infection is usually characterized by fever, which is followed a few days later by a dry, non-productive cough, and shortness of breath. Death from progressive respiratory failure occurs in about 3% to nearly 10% of cases (Poutanen et al., 2003; Lee et al., 2003; Tsang et al., 2003). Attempts to identify the etiology of the SARS outbreak were successful during the third week of March 2003, when laboratories in the United States, Canada, Germany, and Hong Kong isolated a novel coronavirus (SARS-CoV) from SARS patients. Unlike other human coronaviruses, it was possible to propagate SARS-CoV in Vero cells. Evidence of SARS-CoV infection has now been documented in SARS patients throughout the world. SARS-CoV RNA has frequently been detected in respiratory specimens, and convalescent-phase serum specimens from SARS patients contain antibodies that react with SARS-CoV. There is strong evidence that this new virus is etiologically linked to the outbreak of SARS (Ksiazek et al., 2003; Peiris et al., 2003; Drosten et al., 2003). The sequence of two isolates has been reported recently (Rota et al., 2003; Marra et al., 2003). Phylogenetic analyses and sequence comparisons showed that SARS-CoV is not closely related to any of the previously characterized coronaviruses.

Systems and Methods for Reducing the Effects of SARS Infections

Some embodiments of the present invention relate to the use of vectors carrying the SARS-CoV M, E and S genes to induce an immune response. In a preferred embodiment, the vectors induce a response of both arms of the human immune system, the humoral and the cellular parts, but vectors which induce only one arm are also beneficial and are specifically contemplated in some embodiments of the present invention. The immunogenic preparations described herein are safe since there is no chance that dangerous SARS-CoV can be generated from the genes used to form the immunogen. In some embodiments, the DNA is delivered to the cells of a human being where the SARS viral proteins are expressed. The viral proteins produced from the SARS M, E and S genes spontaneously form VLPs which are secreted from the cell just as the virus is secreted during infection. The extracellular presence of the antigen induces the expression of an antibody response thus effectively preparing the immune system for the SARS virus infection. In one embodiment, delivery of the immunogenic SARS-CoV genes induces a cellular immune response.

Plasmid Vectors

One vector which may be used to produce SARS-CoV-VLPs is plasmid DNA. A number of plasmids are suitable for the production of immunogens such as SARS-CoV-VLPs. In general, plasmids used for generating immunogens possess cloning sites for insertion of the DNA used to produce the antigen, DNA sequences necessary for plasmid replication, marker genes for selection in a host cell, such as a bacterial cell, a promoter/enhancer that facilitates the expression of the antigen in eukaryotic cells and a polyadenylation signal. A number of such plasmids are commercially available and most have some or all of these common features. In order to minimize the possibility for chromosomal integration, homology of plasmid DNA sequences to sequences in the human genome is preferably limited. Expression of the antigen(s) can be driven by any suitable promoter or promoter/enhancer combination. For example, in some embodiments, expression is driven by the promoter/enhancer for the immediate early genes of cytomegalovirus (CMV) or the promoter from the Rous sarcoma virus (RSV) long terminal repeat (LTR). In some embodiments, the kanamycin resistance gene may be used for the selection of E. Coli harboring the respective plasmid DNA(s), but any suitable selectable marker can be used. The use of beta-lactam antibiotics, for example, ampicillin is not recommended because of reports of allergic reactions in some individuals. In some vectors, replication of the plasmid DNA(s) in the bacterial hosts is regulated by the pMB1 (ColE1) origin of replication; however, any suitable basic vector origin may be used.

In some embodiments of the present invention, pVAX1 (Invitrogen, Carlsbad, Calif.), which has been specifically designed for the use in the development of DNA vaccines, can be used for the expression of the SARS-CoV M, E and/or S polypeptides. The construction of pVAX1 is consistent with respective guidelines of the Food and Drug Administration (FDA, 1996). Furthermore, this vector's small size makes it suitable for the subcloning of multiple antigen-producing cDNAs in one construct.

It will be appreciated that this invention is not restricted to pVAX1. Other vectors, both plasmid and non-plasmid, can be used. Other exemplary plasmid vectors include, but are not limited to, RapidVACC and pDNA-VACC (Nature Technology Corporation, Lincoln, Nebr.) as well as other eukaryotic expression vectors such as pSVL and pKSV-10 (Pharmacia), pBPV-1/pML2d (International Biotechnologies, Inc.), and pTDT1 (ATCC, #31255).

Viral Vectors

Typically, live attenuated RNA viruses are highly efficient for the purpose of eliciting an immunogenic response. Very successful live attenuated RNA viral vectors include, but are not limited to, Sabin poliovirus, Schwarz measles virus (MV) and the 17D strain of yellow fever virus. The use of these viruses as vaccines has led to a dramatic reduction of the corresponding infections and of their associated pathologies.

For the purpose of vaccination, attenuated RNA viral vectors have a longstanding safety and efficacy record. Additionally, these vectors are easy to produce, inexpensive, and enjoy a wide-ranging system of distribution. When used to generate an immunogenic response, attenuated measles virus induces a strong, life-long humoral and cellular immunity after a single low-dose injection. The MV genome is very stable and reversion of the virus to a pathogenic state has never been observed. MV replicates exclusively in the cytoplasm, and therefore, its genome is never integrated in host DNA. Furthermore, infectious cDNA clones corresponding to the genome of the Edmonston and Schwarz/Moraten strains of MV have been established. A procedure for rescuing the corresponding virus has also been established (EMBO J. 14 5773-5784, 1995). cDNA of up to 5 kb in length have been successfully expressed in these vectors. Accordingly, live attenuated, recombinant MV viral vectors are potentially good vectors for use in eliciting an immunogenic response against both measles and SARS in human populations.

As an alternative to plasmid DNA recombinant viruses can be used as vectors to deliver one or more SARS-CoV genes of interest. For example, in some embodiments of the present invention, recombinant measles viruses are used. Cloned cDNAs, which are prepared as described herein, can be used to generate recombinant measles viruses. In some embodiments of the present invention, the recombinant viruses carry only one of the SARS-CoV cDNAs. In other embodiments, the recombinant viruses carry two or more of the cDNAs which encode the M, S or E polypeptides.

In some embodiments of the present invention, cDNAs corresponding to one or more of the SARS-CoV M, E or S genes are cloned into the pMeasles virus vector, which represents a recombinant cDNA plasmid form of the genomic RNA of the measles virus. Recombinant viruses can be generated from these plasmids by standard rescue experiments (Takeda et al., 2000) in tissue culture.

It will be appreciated that the viral-based embodiments of this invention are not restricted to the use of MV vectors. Other exemplary viral vectors include, but are not limited to, retroviral, adenoviral, adeno associated viral, and lentiviral vectors.

Prokaryotic Vectors

Live attenuated bacteria permit an alternative method for antigen delivery and immunogenic stimulation via the mucosal surfaces and specific targeting to antigen presenting cells located at the inductive sites of the immune system. One approach exploits attenuated intracellular bacteria as a delivery system for eukaryotic antigen expression vectors. Candidate carrier bacteria include, but are not limited to, attenuated strains of Salmonella, Shigella and Listeria species. Certain members of these species have been previously shown to deliver DNA encoding immunogenic antigens to human cells. Delivery of antigen encoding DNA and generation of an immunogenic response, has been demonstrated to be efficacious in several experimental animal models of infectious diseases and tumors.

To be effective, live attenuated prokaryotic strains should maintain a balance between attenuation and immunogenicity. Such strains do not cause any disease or impair normal host physiology, and are at the same time able to colonize the intestine and gut associated lymphoid tissue upon oral administration or other lymphoid organs upon administration by some other route so as to be immunogenic. As antigen carriers, the recombinant Salmonella have been shown to be particularly useful in live vaccines (For review see Curtiss et al. in Essentials of Musocal Immunology, Kagnoff and Kiyono, Eds., Academic Press, San Diego, 1996, pp. 599-611; Doggett and Brown, in Mucosal Vaccines, Kiyono et al., Eds., Academic Press, San Diego, 1996 pp 105-118; see also Hopkins et al. Infect Immun. 63:3279-3286, 1995; Srinavasin et al Vaccines 95, R. N. Chanock et al., Eds., Cold Spring Harbor Laboratory-Press, Plainview, N.Y., p 273-280, 1995). Attenuated strains of Salmonella typhi have been used as human vaccines against typhoid fever as well as against heterologous antigens when used as recombinant antigen delivery vehicles (Forrest, in CRC Press Inc., 1994, pp. 59-80; Levine et al, in New Generation Vaccines Woodrow and Levine, Eds., Marcel Dekker, Inc., New York, 1990, pp. 269-287).

In some embodiments of the present invention, attenuated Salmonella typhi are used to deliver desired genes encoding SARS-CoV antigenic proteins to humans. The method comprises selecting a strain of bacteria such as Salmonella typhi having, (i) an inactivated pro-apoptotic gene, (ii) an inactivated vacuole retaining gene, (iii) one or more inactivating mutations which render the strain attenuated, and (iv) a recombinant gene(s) encoding the SARS-CoV S—, M- and E-polypeptides. The strain is then administered to the human. The one or more inactivating mutations which render the strain attenuated can involve a mutation in one gene or a mutation in each of two or more genes. Additionally, the attenuated Salmonella contain at least one recombinant gene capable of expressing SARS-CoV genes which allows their use as carriers or delivery vehicles of the gene product to subjects, such as humans. By delivery of the desired gene it is meant that a nucleic acid, either DNA or RNA, encoding the SARS-CoV products is delivered to the subject. The Salmonella strains can also deliver RNA corresponding to virus replicons or infectious, attenuated viruses such as, but not restricted to, Sabin poliovirus, yellow fever virus 17D or measles virus. When the viral RNA is delivered in the cytoplasm of the infected cells replication of the virus or replicon starts and as a consequence of the replication the antigenic sequences are further amplified and better expression is observed

In some embodiments of the present invention, the use of Salmonella typhi facilitates invasion and colonization of any of the gut associated lymphoid tissues (GALT), nasal associated lymphoid tissue (NALT) or the bronchial associated lymphoid tissue (BALT) which is collectively called the mucosal associated lymphoid tissue (MALT). Among the several advantages achieved by the use of Salmonella typhi as delivery system is the fact that the bacteria are capable of colonizing and delivering a desired SARS-CoV gene product to the gut associated lymphoid tissue if administered orally, to the nasal associated lymphoid tissue if administered intranasally and to other lymphoid organs if administered by other routes. Additionally, the use of attenuated Salmonella typhi is an efficient and inexpensive method for delivery of a nucleic acid molecule to human cells.

In some embodiments of the present invention, the attenuated Salmonella are able to colonize Peyer's patches or similar tissues which include, for example, other lymphoid tissues of the GALT in humans, without destroying the invaded cells. This action provides a high immunogenicity upon oral administration. Furthermore, the M cells of the follicle-associated lymphoid tissue of the GALT are functionally, morphologically and structurally the same as the M cells associated with other mucosal associated lymphoid tissues (MALT) in the body, such as conjunctiva associated lymphoid tissue (CALT), bronchus associated lymphoid tissue (BALT) and nasal associated lymphoid tissue (NALT), as well as lymphoid tissues in the rectum, an the like. As such, Salmonella is capable of invading and colonizing all of these tissues when administration is by the appropriate route, for example, oral, intranasal and rectal.

Induction of an Immune Response to SARS-CoV-VLPs

In some embodiments of the present invention, an immune response to SARS-CoV-VLPs is induced in an subject at risk for developing SARS. Such subjects include animals such as birds and mammals. Some embodiments of the present invention relate to the induction of an immune response to SARS-VLPs in chickens and other fowl. In some embodiments, an immune response to SARS-CoV-VLPs is induced in mammals including, but not limited to, mice, rats, cats, dogs, pigs, cows, horses, goats, sheep and monkeys. In a preferred embodiment, an immune response to SARS-CoV-VLPs is induced in humans.

It is contemplated that virtually any type of vector, including naked DNA in the form of a plasmid or other nucleic acid vector, can be employed to generate an immune response in conjunction with a wide variety of immunization protocols including, but not limited to, parenteral, mucosal and gene-gun inoculations.

The preparation of immunogens as well as genetic constructs encoding immunogens as active ingredients is generally well understood in the art, as exemplified by U.S. Pat. Nos. 6,716,823; 5,703,057; 4,608,251; 4,601,903; 4,599,231; 4,599,230; 4,596,792; and 4,578,770, the discloses of which are incorporated herein by reference in their entireties. Typically, SARS-CoV-VLPs, genetic constructs encoding SARS-CoV polypeptides or portions thereof which can form SARS-CoV-VLPs and/or prokaryotic vectors comprising such genetic constructs are prepared as injectables, either as liquid solutions or suspensions, or as solid forms suitable for solution or suspension in liquid prior to injection. Such preparations may also be emulsified. The active immunogenic ingredient(s) is often mixed with excipients which are pharmaceutically acceptable and compatible with the active ingredient(s). Suitable excipients are, for example, water, saline, dextrose, glycerol, ethanol, or the like and combinations thereof. In addition, if desired, the preparation may contain minor amounts of auxiliary substances such as wetting or emulsifying agents, pH buffering agents, or adjuvants which enhance the immunogenic potential of the preparation.

Immunogenic preparations may be conventionally administered parenterally, by injection, for example, either subcutaneously or intramuscularly. Additional formulations which are suitable for other modes of administration include powders for nasal administration, oral formulations and suppositories.

Powders for nasal administration are prepared by suspending insoluble nucleic acid constructs or VLPs in an aqueous solution of the hydrophilic excipient and drying the solution to produce a powder comprising particles of the nucleic acid construct or VLPs dispersed within the dried excipient material, usually in the presence of excess powdered excipient. The weight ratio of nucleic acid construct or VLP to hydrophilic excipient in the initial solution is any ratio consistent with the intended use. Preferably the weight ratio of nucleic acid construct or VLP to hydrophilic excipient in the initial solution is from 1:1 to 1:10. The solution may be dried by spraying droplets into a flowing gas stream (spray drying) or by vacuum drying to produce a crude powder followed by grinding to produce a final powder. In the case of particles intended for lung delivery, particles having a size from 0.5 μm to 5 μm are desirable.

Oral formulations include such normally employed excipients as, for example, pharmaceutical grades of mannitol, lactose, starch, magnesium stearate, sodium saccharine, cellulose, magnesium carbonate and the like. These compositions can take the form of solutions, suspensions, tablets, pills, capsules, sustained release formulations or powders and, in some embodiments, contain about 10 to about 95% of active ingredient, preferably about 25 to about 70%.

For suppositories, traditional binders and carriers may include, for example, polyalkalene glycols or triglycerides. Such suppositories may be formed from mixtures containing the active ingredient. In some embodiments, the suppositories are formed from mixtures containing the active ingredient in the range of about 0.5% to about 10%, preferably about 1 to about 2%.

In some embodiments of the present invention, immunogenic preparations are administered in a manner compatible with the dosage formulation and in such amount as to be immunogenic and therapeutically effective. The quantity to be administered depends on the subject to be treated, including, for example, the capacity of the subject's immune system to synthesize antibodies, and the degree of protection desired. Precise amounts of active ingredient required to be administered depend on the judgment of the practitioner. However, in preferred embodiments, suitable dosage ranges are of the order of several hundred micrograms active ingredient per dose. Suitable regimes for initial administration and booster doses are also variable, but are typified by an initial administration followed by subsequent inoculations or other administrations.

In many instances, it will be desirable to have multiple administrations of the immunogenic preparations. In some embodiments, administration will normally be at from two to twelve week intervals but more usually from three to five week intervals. Periodic boosters at intervals of 1-5 years, usually three years, are desirable to maintain protective levels of immunogenic response. The course of the immunization can be followed by assays for antibodies to the target antigens. The assays can be performed by labeling with conventional labels, such as radionuclides, enzymes, fluorescents, and the like. These techniques are well known and may be found in a wide variety of patents, such as U.S. Pat. Nos. 3,791,932; 4,174,384 and 3,949,064, the disclosures of which are incorporated herein by reference in their entireties.

### EXAMPLES

Some embodiments of this invention are further illustrated by the following examples which should not be construed as limiting. It will be appreciated by those of skill in the art that the techniques disclosed in the examples which follow represent techniques that function well in the practice of the embodiments of the invention described herein, and thus, can be considered to constitute preferred modes for the practice of these embodiments. Those of skill in the art will, however, in light of the present disclosure, will appreciate that many changes can be made in the specific embodiments which are disclosed herein and still obtain a like or similar result without departing from the spirit and scope of the invention.

### Example 1

### Comparison of SARS-CoV M, E and S Polypeptides from Four SARS Isolates

An analysis of the relationship between the M, E and S polypeptides of known pathogenic strains of SARS-CoV was performed to determine the potential for developing a broadly applicable SARS-CoV-VLP that would be useful in producing an immunogenic response to most or all the isolates of SARS-CoV.

SARS-CoV isolates used in this analysis were Tor2, Urbani, HKU-39849 and CUHK-W1. SARS-CoV strain Tor2 was isolated at the Genome Sciences Centre, British Columbia Cancer Research Centre, 600 West 10th Avenue, Vancouver, BC V5Z 4E6, Canada. The sequence of the genome was determined and then deposited in Genbank under the Accession Number: AY274119 (complete genome, 29751 bp) (SEQ ID NO: 14), the disclosure of which is incorporated herein by reference in its entirety. SARS-CoV strain Urbani was isolated at the Division of Viral and Rickettsial Diseases, Centers for Disease Control and Prevention, 1600 Clifton RD, NE, Atlanta, Ga. 30333, USA. The sequence of the genome was determined and then deposited in Genbank under the Accession Number: AY278741 (complete genome, 29727 bp) (SEQ ID NO: 15), the disclosure of which is incorporated herein by reference in its entirety. SARS-CoV strain HKU-39849 was isolated at the Department of Zoology, The University of Hong Kong, Pokfulam Road, Hong Kong, HK 00000, China. The sequence of the genome was determined and then deposited in Genbank under the Accession Number: AY278491 (complete genome, 29742 bp) (SEQ ID NO: 16), the disclosure of which is incorporated herein by reference in its entirety. SARS-CoV strain CUHK-W1 was isolated at the Department of Biochemistry, Chinese University of Hong Kong, MMW Bldg. Rm 608, Shatin, NT SAR, China. The sequence of the genome was determined and then deposited in Genbank under the Accession Number: AY278554 (complete genome, 29736 bp) (SEQ ID NO: 17), the disclosure of which is incorporated herein by reference in its entirety.

Additional strains of SARS-CoV have been isolated but their genomes have been only partially sequenced. Such strains were not included in this analysis but are described as follows: SARS-CoV strains BJ01, BJ02, BJ03, BJ04 were isolated at the Institute of Microbiology and Epidemiology, Academy of Military Medical Sciences/Beijing Genomics Institute, Chinese Academy of Sciences, Beijing 101300, China. The genomes of each of these strains were partially sequenced and deposited in Genbank under the Accession Numbers: AY278488, AY278487, AY278490, AY279354, respectively, the disclosures of which are incorporated herein by reference in their entireties. SARS-CoV strain GZ01 was isolated at the Institute of Microbiology and Epidemiology, Academy of Military Medical Sciences/Beijing Genomics Institute, Chinese Academy of Sciences, Beijing, Beijing 101300, China. The genome of this strain was partially sequenced and deposited in Genbank under the Accession Number: AY278489, the disclosures of which is incorporated herein by reference in its entirety.

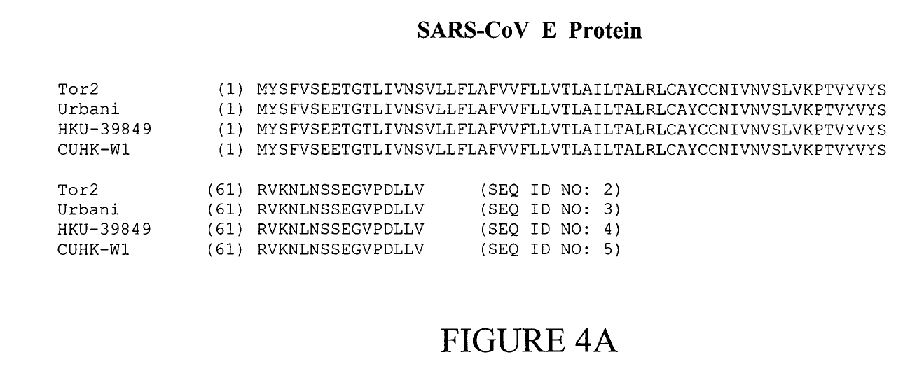

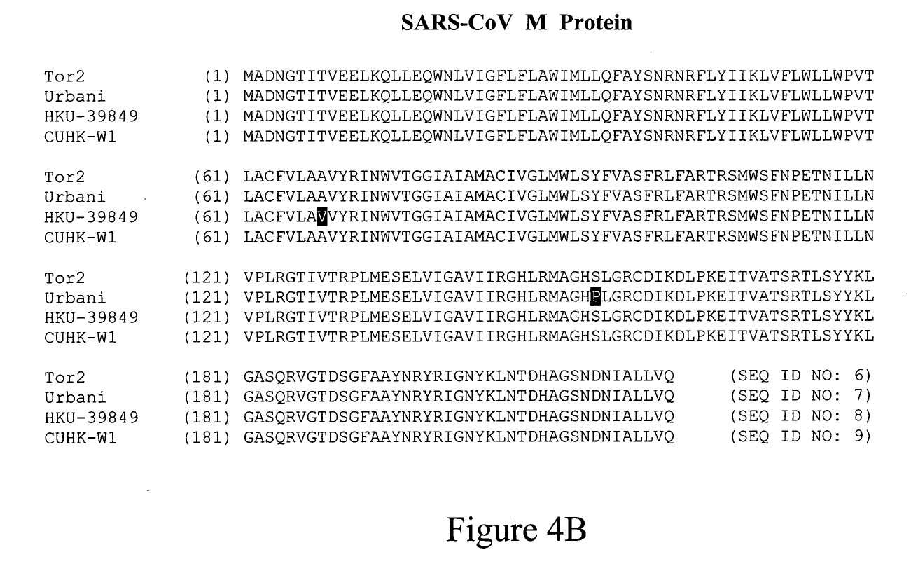

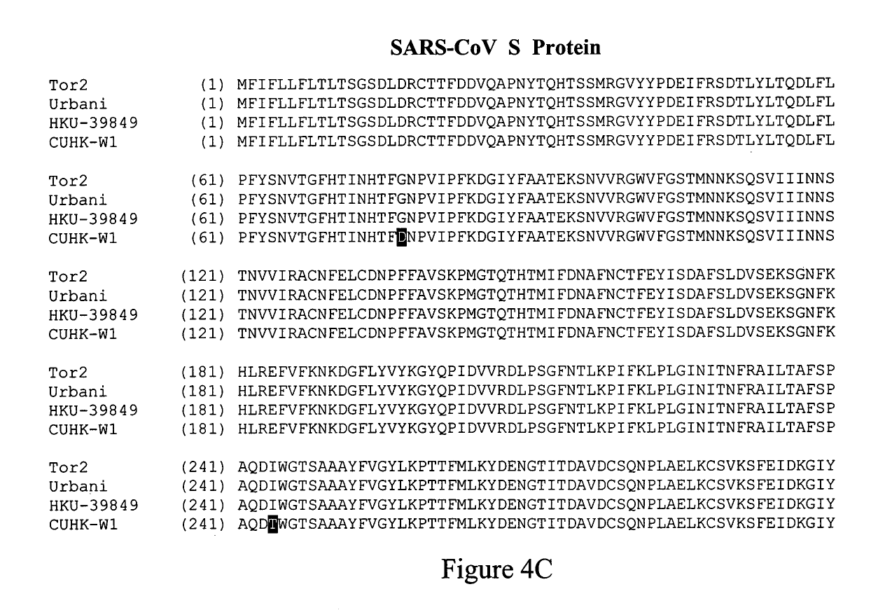

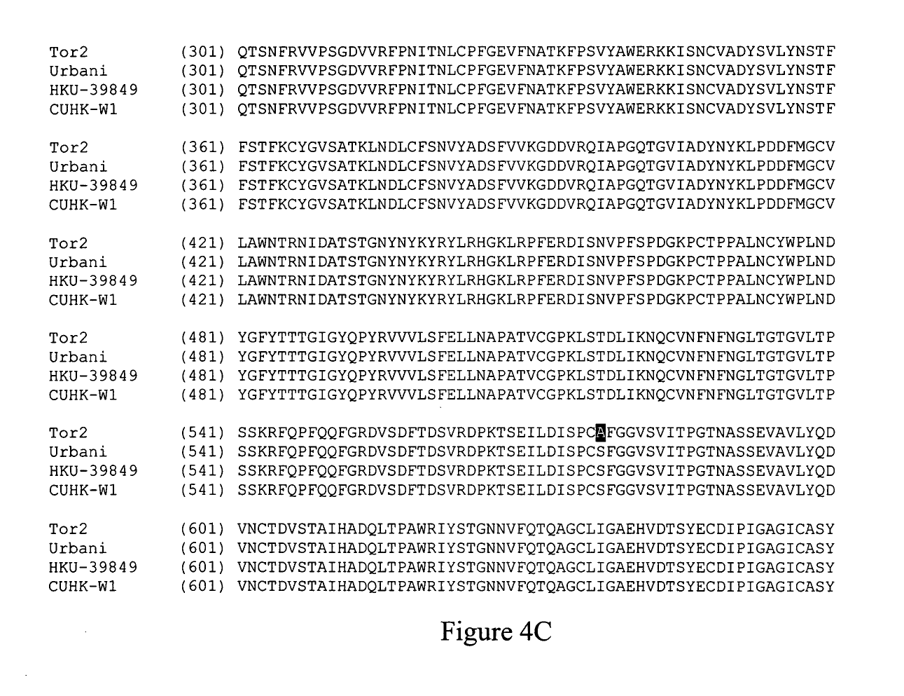

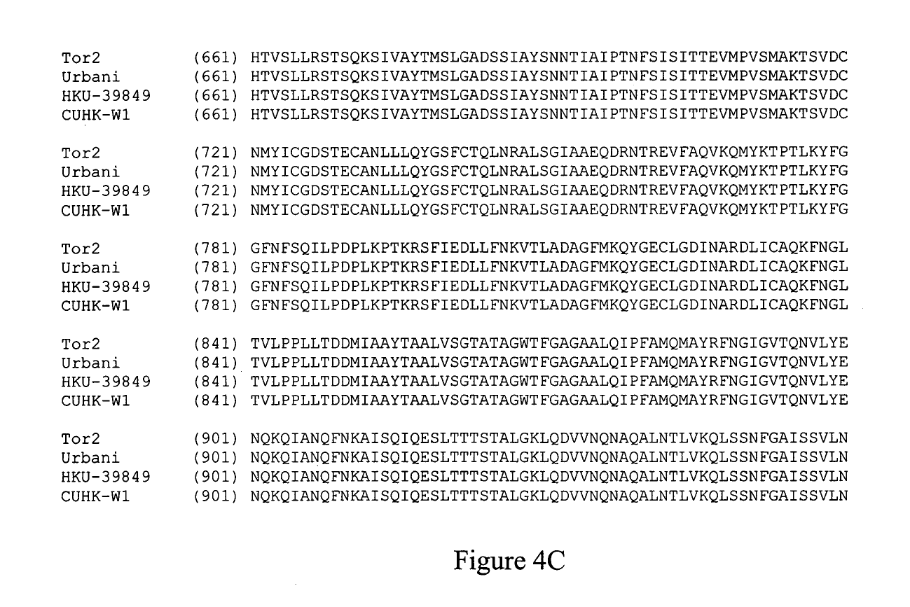

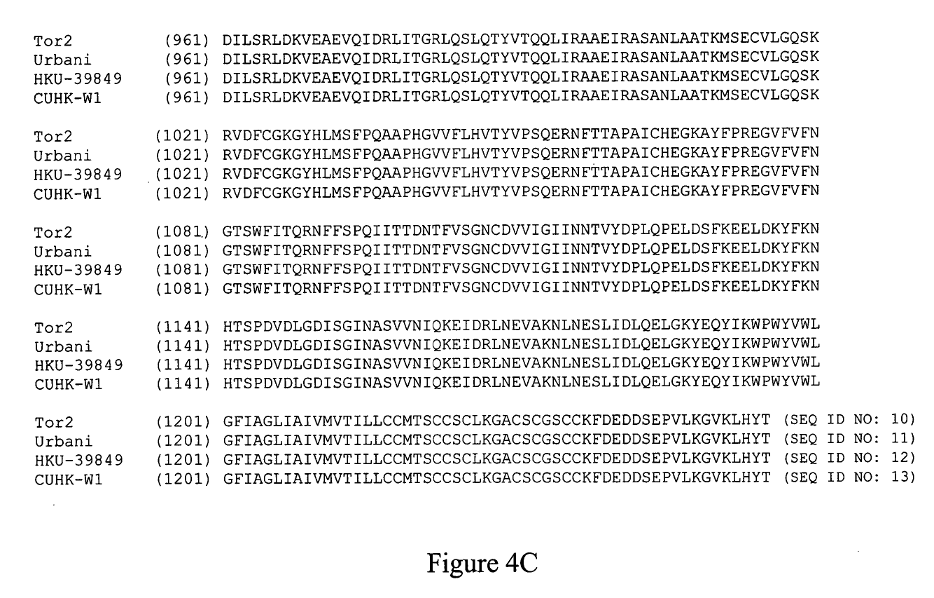

The amino acid sequences for the M, E and S polypeptides were determined from the genomic sequence by ORF analysis and homology comparison with other known coronaviruses. For each of the four strains, the sequences of the M, E and S polypeptides were compared using the sequence alignment program AlignX with a blosum62mt2 matrix, an opening penalty of 10 and a gap extension penalty of 0.05. Sequence alignments for the E, M and S polypeptides are shown in FIG. 4A-C, respectively.

The results of the sequence alignments show that the M, E and S polypeptides are highly conserved among the four SARS-CoV strains tested here. Comparison of the E polypeptide sequence shows that the sequences are 100 percent identical for each of the four SARS-CoV isolates (see FIG. 4A). The M polypeptide sequence shows only minor variation among the four strains. In particular, strain HKU-39849 differs from the other strains by having valine at position 67 rather than alanine. Strain Urbani differs from the other strains at position 154 by containing proline rather than serine (see FIG. 4B). The S polypeptide also shows only minor variation. In particular, strain CUHK-W1 contains aspartate rather than glycine at position 77 and threonine rather than isoleucine at position 244. Strain Tor2 contains an alanine at position 577 rather than a serine (see FIG. 4C).

The result of the sequence comparisons indicate that a VLP formed from the M, E and S polypeptide from anyone one of the strains tested here would generate an immune response specific not only to the strain from which the VLP was constructed but to a number of different SARS-CoV strains.

While some embodiments of the present invention are directed to VLPs constructed using the genes from a single SARS-CoV strain, it will be appreciated that other embodiments contemplate the use of genes from a plurality of SARS-CoV strains to generate VLPs. Additionally, other embodiments of the present invention, are directed to mixtures comprising VLPs corresponding to a plurality of SARS-CoV strains, wherein each VLP is produced by using the genes of a single SARS-CoV strain.

### Example 2

### Preparation of SARS-CoV cDNA

In this Example and the Examples that follow below, SARS-CoV strain Urbani cDNAs are reversely transcribed from an RNA preparation of the SARS-CoV strain Urbani genomic RNA. Methods for reverse transcription are well known in the art. The prepared cDNA is used as a template for PCR reactions and other applications described herein. Table 2 lists the sequence identification numbers (SEQ ID NO) for the cDNAs used herein.

TABLE 2

cDNAs Used in Plasmid Construction

| | |
|---|---|
| cDNA | SEQ ID NO: |

| | |
|---|---|
| cDNA for S-protein | SEQ ID NO: 18 |
| cDNA for M-protein | SEQ ID NO: 19 |
| cDNA for E-protein | SEQ ID NO: 20 |
| cDNA for IRES | SEQ ID NO: 21 |
| cDNA for RSV-LTR Promoter | SEQ ID NO: 22 |
| cDNA for SV40 Polyadenylation Signal | SEQ ID NO: 23 |

### Example 3

### Preparation of a Plasmid for the Expression of SARS-CoV M, E and S Polypeptides

In this Example, a single plasmid for the expression of all three SARS-CoV polypeptides is constructed.

A 411 bp DNA fragment containing the Rous sarcoma virus (RSV) long terminal repeat (LTR) promoter is amplified by the polymerase chain reaction (PCR) (Sambrook et al., 2001) using the following primers:

(SEQ ID NO: 24)

| | |
|---|---|
| RSVBACK: | 5′-AATAACTGCAGCGATGTACGGGCCAGATATAC-3′; |
| and | |

(SEQ ID NO: 25)

| | |
|---|---|
| RSVFOR: | 5′-AATAAGCGGCCGCGGAGGTGCACACCAATGTGG-3′. |

The primers are engineered such that, a PstI-site will be present in the 5′-end of the resulting PCR product and a NotI-site will be present in the 3′-end. These restriction sites permit the RSV-LTR promoter to be inserted into the unique PstI- and NotI-sites in the multiple cloning site of pVAX1 (Invitrogen, Carlsbad, Calif.) (SEQ ID NO: 26).

In addition to the RSV-LTR promoter, a 240 bp DNA fragment harboring the simian virus (SV) 40 polyadenylation signal is amplified by PCR using the following primers:

(SEQ ID NO: 27)

| | |
|---|---|
| SV40BACK: | 5′-TTATTAAGCTTATGTACTCATTCGTTTCGGAAG-3′; |
| and | |

(SEQ ID NO: 28)

| | |
|---|---|
| SV40FOR: | 5′-TATTGGTACCGACCAGAAGATCAGGAACTCC-3′. |

Amplification using the above primers adds a HindIII-site to the 5′-end and a KpnI-site to the 3′-end of the resulting SV40 polyadenylation signal. This PCR fragment is then inserted into the unique HindIII and KpnI-sites of the pVAX1-derivative, which already contains the RSV-LTR promoter. The resulting plasmid is referred to as pCSRB.

A 3792 bp cDNA fragment (SARS_S) encoding for the spike (S) glycoprotein of SARS-CoV strain Urbani (Genbank Accession No: AY278741) is amplified by PCR using the following primers:

| | |
|---|---|
| SARS-S-BACK: | |
| 5′-TAATATCTAGAGCCGCCGCCATGTTTATTTTCTTATTATTTCTTACTCTCAC-3′; | (SEQ ID NO: 29) |
| and | |
| SARS-S-FOR: | |
| 5′-TAATAGTTTAAACTTATCATGTGTAATGTAATTTGACACCC3′. | (SEQ ID NO: 30) |

Using the above primers, an XbaI-site and a Kozak (Kozak, 1987) consensus sequence are engineered into to the 5′-end of the SARS_S cDNA PCR product. The Kozak sequence is a short recognition sequence, which is found in most eukaryotic mRNAs, that greatly facilitates initial mRNA binding to the small subunit of the ribosome. The consensus sequence for initiation of translation in vertebrates is (GCC)GCC^{A}/_{G}CCATGG (SEQ ID NO: 31). At the 3′-end of the SARS_S cDNA two translation termination codons and a PmeI-site are added thereby allowing insertion of this sequence as an XbaI/PmeI-fragment into the unique XbaI- and PmeI-sites downstream of the RSV-LTR promoter in pCSRB.

Finally, cDNAS encoding for the SARS-CoV M polypeptide (SARS_M cDNA) and E polypeptide (SARS_E cDNA) and a fragment containing the Mahoney strain poliovirus Type 1 internal ribosome entry site (IRES) are amplified by PCR using the following primers:

| | |
|---|---|
| SARS-M-BACK: | (SEQ ID NO: 32) |

5′-TAATAGCTAGCGCCGCCGCCATGGCAGACAACGGTACTATTAC-3′;

| | |
|---|---|
| SARS-M-FOR1: | (SEQ ID NO: 33) |

5′-GAGCTGTTTTAATCATTACTGTACTAGCAAAGCAATATTGTC-3′;

| | |
|---|---|
| IRES-BACK: | (SEQ ID NO: 34) |

5′-GCTAGTACAGTTAAAACAGCTCTGGGGTTGTAC-3′;

| | |
|---|---|
| IRES-FOR: | (SEQ ID NO: 35) |

5′-CGAATGAGTACATTATGATACAATTGTCTGATTG-3′;

| | |
|---|---|
| SARS-E-BACK1: | (SEQ ID NO: 36) |

5′-TTGTATCATAATGTACTCATTCGTTTCGGAAG-3′;
and

| | |
|---|---|
| SARS-E-FOR1: | (SEQ ID NO: 37) |

5′-TATTACTTAAGTTATCAGACCAGAAGATCAGGAACTCC-3′.

The primers are designed so as to introduce ˜25 bp overlaps between the 3′-end of SARS_M cDNA and the 5′-end of IRES as well as between the 3′-end of IRES and the 5′-end of SARS_E CDNA. Additionally, an NheI-site and a Kozak consensus sequence are engineered into the 5′-end of the SARS_M cDNA product. Two translation termination codons are added to the 3′-end of the SARS_M cDNA product and two translation termination codons and an AflII-site are introduced at the 3′-end of the SARS_E cDNA product. In a final PCR reaction, these three fragments are ligated by overlap extension resulting in a fragment referred to here as M_IRES_E. Using restriction enzymes NheI and AflII, M_IRES_E is positioned downstream of the CMV promoter in the pCSRB-derivative which already contains the SARS-S cDNA. The resulting construct is referred to as pMES.

### Example 4

### Preparation of a Plasmid for the Expression of SARS-CoV M and E Polypeptides

In this Example, a plasmid for the expression of the SARS-CoV M and E polypeptides is constructed using pVAX1. The resulting plasmid is named pME. pME is constructed by inserting a bicistronic construct that encodes for the M and E proteins from SARS-CoV into the polylinker region of pVAX1. The M/E bicistronic construct is made by reamplifying the M_IRES_E fragment from pMES (see Example 3) using the following primers:

| | |
|---|---|
| SARS-M-BACK: | (SEQ ID NO: 38) |

5′-TAATAGCTAGCGCCGCCGCCATGGCAGACAACGGTACTATTAC-3′;
and

| | |
|---|---|
| SARS-E-FOR2: | (SEQ ID NO: 39) |

5′-TATTAGTTTAAACTTATCAGACCAGAAGATCAGGAACTCC-3′.

Amplification with the SARS-M-BACK and SARS-E-FOR2 generate an M_IRES_E fragment having an NheI-site is engineered into its 5′-end and a PmeI-site included at its 3′-end. These sites permit the fragment to be inserted into the unique NheI- and the 3′-most PmeI-sites in the multiple cloning site of pVAX1 downstream of the CMV promoter, thereby producing pME.

### Example 5

### Preparation of a Plasmid for the Expression of SARS-CoV S Polypeptide

In this Example, a plasmid for the expression of the SARS-CoV S polypeptide is constructed using pVAX1. This construct is named pS. pS is constructed by inserting the SARS_S cDNA (generated as in Example 3) as an XbaI/PmeI-fragment into the NheI- and PmeI-sites downstream of the CMV promoter in pVAX1. Note that XbaI and NheI produce compatible overhangs.

### Example 6

### Preparation of a Plasmid for the Expression of SARS-CoV M Polypeptide

In this Example, a plasmid for the expression of the SARS-CoV M polypeptide is constructed using pVAX1. This construct is named pM. To generate pM, the SARS_M cDNA is amplified by PCR using the following primers:

| | |
|---|---|
| SARA-M-BACK: | (SEQ ID NO: 40) |

5′-TAATAGCTAGCGCCGCCGCCATGGCAGACAACGGTACTATTAC-3′;
and

| | |
|---|---|
| SARS-M-FOR2: | (SEQ ID NO: 41) |

5′-TAATAGTTTAAACTCATTACTGTACTAGCAAAGCAATATTGTC-3′.

The SARS_M cDNA product is then ligated as an NheI/PmeI-fragment into the multiple cloning site of pVAX1 downstream of the CMV promoter, thereby producing plasmid, pM.

### Example 6

### Preparation of a Plasmid for the Expression of SARS-CoV E Polypeptide

In this Example, a plasmid for the expression of the SARS-CoV E polypeptide is constructed using pVAX1. This construct is named pE. To generate pE, the SARS_E cDNA is amplified by PCR using the following primers:

| | |
|---|---|
| SARS-E-BACK2: | (SEQ ID NO: 42) |

5′-TTATTGCTAGCATGTACTCATTCGTTTCGGAAG-3′;
and

| | |
|---|---|
| SARS-E-FOR2: | (SEQ ID NO: 43) |

5′-TATTAGTTTAAACTTATCAGACCAGAAGATCAGGAACTCC-3′.

The SARS_E cDNA product is then ligated as an NheI/PmeI-fragment into the multiple cloning site of pVAX1 downstream of the CMV promoter, thereby producing plasmid, pE.

### Example 7

### Preparation of a Virus for the Expression of SARS-CoV Polypeptides

This Example describes the construction of recombinant measles virus (MV) expressing SARS-CoV genes.

MV recombinant plasmids are derived from plasmid p(+)MV, which carries the antigenomic MV tag Edmonston B or Schwarz/Moraten vaccine strain of MV sequence. Two additional transcription units (ATU) containing unique restriction sites for insertion of open reading frames (ORFs) are introduced in MV cDNAs. A first ATU is located downstream the P gene and a second ATU is located downstream the H gene. These engineered plasmids are used for inserting the SARS-CoV genes which encode the M and E polypeptides.

The SARS-CoV cDNAs encoding the S protein and the M/E proteins (see Example 3) are amplified by PCR using Pfu polymerase and primers that contain unique BsiWI and BssHII sites for subsequent cloning into the MV vectors. The primers also encode artificial start and stop codons. In additional nucleotides are included after the stop codon in order to comply with the “rule of six,” which requires that the number of nucleotides of the MV genome must be a multiple of six. SARS CoV structural genes are introduced in the pMV vector in the first and second ATU sites.

Recombinant MV-SARS-CoV viruses are recovered from plasmids using the helper-cell-based rescue system, described by Radecke et al. (EMBO J. 14 5773-5784, 1995). Briefly, human helper cells stably expressing T7 RNA polymerase and measles N and P proteins are co-transfected using the calcium phosphate procedure with the different pMV/SARS-CoV genes plasmids (5 μg) and a plasmid expressing the MV polymerase L gene. After overnight incubation at 37° C., the transfection medium is replaced by fresh medium and the cells are heat shocked (43° C. for two hours). After two days of incubation at 37° C., transfected cells are transferred onto a 70% confluent Vero cells layer and incubated at 37° C. Syncytia appear in Vero cells after 2-5 days of culture. Single syncytia are transferred to 35-mm-diameter wells of Vero cells. Infected cells are then expanded to T-75 or T-150 flasks. When syncytia reach 80-90%, viruses are harvested by scraping the cells in 3 ml of MEM medium, followed by one round of freezing and thawing. Supernatants are then clarified from cell debris by centrifugation and kept at −80° C.

### Example 8

### Construction of a Prokaryotic Vector for the Expression of SARS-CoV Polypeptides

In this Example, attenuated Salmonella typhi is used to delivery SARS-CoV M, E and S genes. The cloned cDNAs in pS, pM and pE described above are used to generate plasmids carrying different Salmonella-specific origins of replication (ori) as well as selectable markers, e.g. kanamycin. The recombinant bacteria are then tested in tissue culture for the production of the SARS-CoV specific proteins. The attenuated Salmonella typhi strain is then used to deliver the SARS-CoV M, E and S genes to humans.

### Example 9

### Antibodies Against SARS-CoV Polypeptides

This Example describes the production of polyclonal antibodies capable of binding the SARS-CoV M, E and/or S polypeptides. In particular, the amino terminal and carboxy terminal 15 amino acids of each of the mature SARS-CoV M, E and S polypeptides are chemically synthesized and subsequently used to immunize rabbits. The sequences that are used are as follows:

| | | |
|---|---|---|
| S Protein/N-term: | DLDRCTTFDDVQAPN | (SEQ ID NO: 44) |
| S Protein/C-term: | DDSEPVLKGVKLHYT | (SEQ ID NO: 45) |
| M Protein/N-term: | MADNGTITVEELKQL | (SEQ B) NO: 46) |
| M Protein/C-term: | TDHAGSNDNIALLVQ | (SEQ ID NO: 47) |
| E Protein/N-term: | MYSFVSEETGTLIVN | (SEQ ID NO: 48) |
| E Protein/C-term: | VKNLNSSEGVPDLLV | (SEQ ID NO: 49) |

The immunized animals are boosted once a month for a total of three times with 200 μg peptide each time. The resulting sera are used as a tool to detect the M, E and S polypeptides in subsequent experiments.

### Example 10

### Expression of SARS-CoV Polypeptides

The cloning and expression of SARS-CoV polypeptides from each of the constructs described herein are verified in vitro. For plasmid constructs in pVAX1, the bacteriophage T7 promoter facilitates in vitro synthesis of mRNAs encoding the SARS-CoV antigenic polypeptides by T7 RNA polymerase. The mRNAs encoding the SARS-CoV antigens are transcribed in vitro then used as a template in a reticulocyte lysate in vitro translation reaction in the presence of ³⁵S-methionine and/or ³⁵S-cysteine. Radioactively labeled translated SARS-CoV antigens are separated by standard polyacrylamide gel electrophoresis (PAGE) and visualized by fluorography. Alternatively, the radiolabelled antigens are subjected to immunoprecipitation using the antisera generated against the M, E, and/or S polypeptides (see Example 9). Immune complexes are precipitated by protein A or protein G agarose, separated by PAGE and visualized by fluorography.

The functionality of the plasmid constructs described herein can be verified in tissue culture. In such experiments, tissue culture cells are transfected with any combination of genetic constructs which encode the polypeptides necessary to produce SARS-CoV-VLPs. For example, cells can be transfected with pMES; cotransfected with pME and pS; or cotransfected pM, pE, and pS. In such experiments, approximately 2×10⁶ cells are transfected using Lipofectamine (Invitrogen) according to the manufacturers protocol. After 24 hours, the cells are lysed in SDS-containing polyacrylamide gel sample buffer. The proteins are then separated on commercially available 8-20% PAA gels and subjected to western blotting. After transfer to nitrocellulose the rabbit antisera described in the Example 9 are used to detect the SARS-CoV polypeptides expressed in the tissue culture cells.

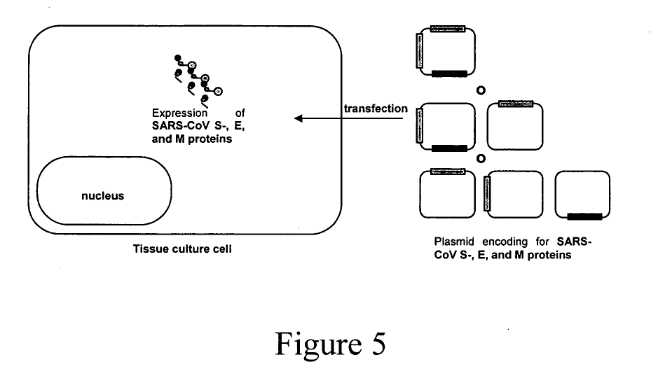

FIG. 5 shows that transfected tissue culture cells produce the SARS-CoV M, E and S polypeptides upon transfection of plasmids carrying these SARS-CoV genes under control of a eukaryotic promoter.

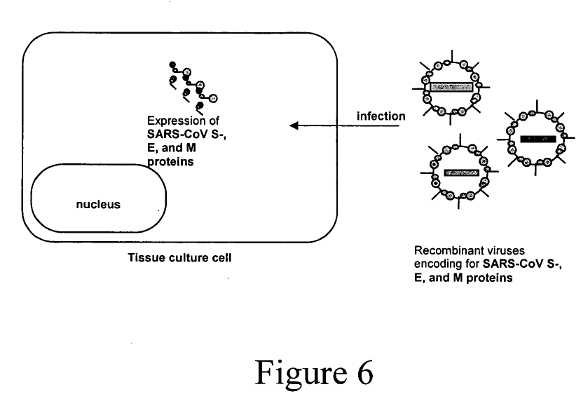

Viral constructs comprising SARS-CoV polypeptides can be used to infect cell cultures and expression of these polypeptides can be tested as described above. FIG. 6 illustrates tissue culture cells producing the SARS-CoV M, E and S polypeptides upon infection with a recombinant measles virus carrying these SARS-CoV genes.

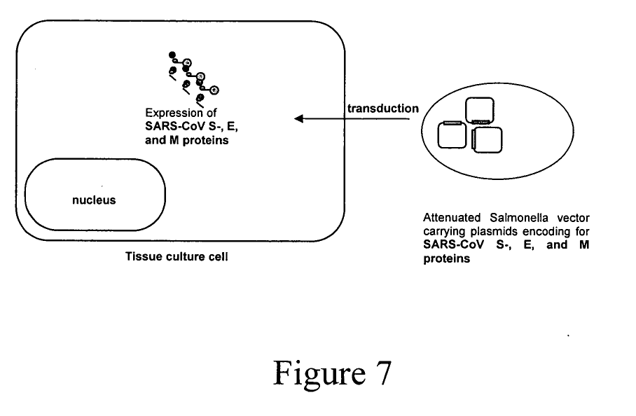

Prokaryotic delivery vector constructs comprising genetic constructs, such as the plasmids described herein, can also be tested for the ability to induce the expression of SARS-CoV proteins in tissue cell culture. FIG. 7 illustrates tissue culture cells which produce SARS-CoV proteins upon transduction by an attenuated Salmonella strain carrying genetic construct(s) having the SARS-CoV M, E and S genes under control of a eukaryotic promoter.

### Example 11

### Production of SARS-CoV-VLPs

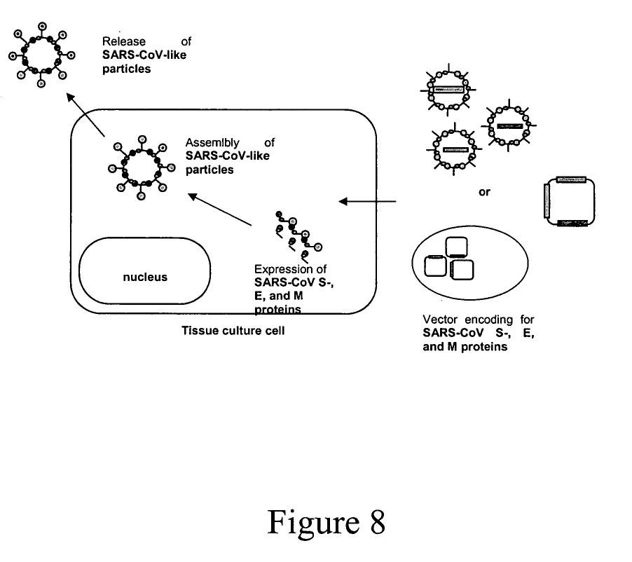

This Example demonstrates that the M, E and S proteins are sufficient to generate SARS-CoV like particles that are secreted from the cell and that the VLP is comprised of all three proteins. Approximately, 2×10⁷ tissue culture cells are transfected with plasmid DNA or infected with recombinant viruses or transduced with recombinant bacteria carrying the SARS-CoV M, E and S genes as described in the previous Example. The cell cultures are incubated for approximately 24 hours then the supernatants are collected. During the 24 hour incubation period, the SARS-CoV polypeptides are produced by the cells, virus-like particles are formed, budding occurs and the VLPs are released by the cells. FIG. 8 illustrates tissue culture cells which produce SARS-CoV-VLPs upon expression of the SARS-CoV M, E and S genes and then release these VLPs into the tissue culture supernatant.

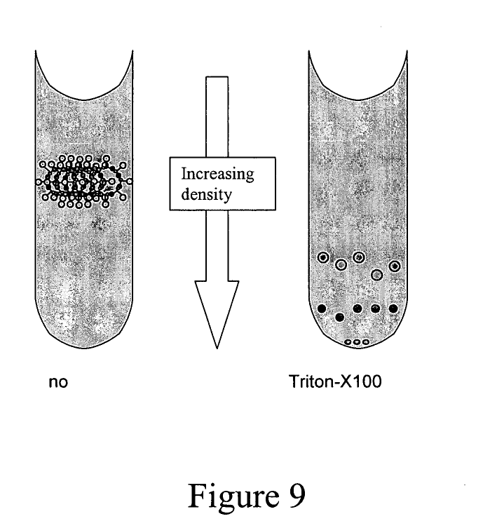

To demonstrate that the M, E and S polypeptides are all part of the VLPs, density centrifugation of untreated and solubilized cell supernatant preparations is used. For solubilized preparations, Triton-X 100 is added to an aliquot of the supernatant to a final concentration of 1% thus effectively solubilizing the viral envelope. After solubilization, the M, E and S polypeptides are free to migrate through the gradient according to their own molecular weight. In contrast, the proteins in untreated material migrate according to the higher molecular weight of the VLP. FIG. 9 illustrates a density gradient centrifugation of intact virus particle not treated with Triton-X100 (no) and the migration of solubilized proteins (Triton-X100) in a sucrose gradient.

Fractions corresponding to individual areas of the gradient are collected and analyzed via western blotting as described above. This result shows: (i) that SARS-CoV-VLPs are produced by eukaryotic cells transfected with the SARS-CoV genes M, E and S; and (ii) that all three of the SARS-CoV polypeptides are part of the SARS-CoV-VLP.

It will be appreciated that SARS-CoV-VLPs can be isolated from tissue culture medium and formulated as an immunogenic pharmaceutical preparation using methods well known in the art. Alternatively, all or a portion of the genes encoding the polypeptides necessary for the production of the SARS-CoV-VLP can be administered to subject via the methods described herein. Expression of the genes within the subject will permit the production of immunogenic SARS-CoV-VLPs.

### Example 12

### Formulation of Vectors for Delivery to a Subject

Formulation of Plasmid Vectors

Plasmid DNA can be prepared for delivery by precipitation using ethanol and collecting the plasmid by centrifugation. Subsequently the plasmid is extensively washed using 70% ethanol and briefly dried. Any suitable formulation may be used to administer the DNA. In one embodiment, the DNA is solubilized in phosphate-buffered saline and can then be used for injection.

As an alternative, plasmid DNA can be formulated as a dry powder. Dry powder nucleic acid compositions include insoluble nucleic acid constructs (typically small particles) dispersed within a matrix of hydrophilic excipient material to form large aerosol particles. The powdered aerosol particles have an average particle size usually in the range from 0.5 μm to 5 μm for lung delivery with larger sizes being useful for delivery to other moist target locations.

Dry powder nucleic acid compositions are prepared by suspending insoluble nucleic acid constructs in an aqueous solution of the hydrophilic excipient and drying the solution to produce a powder comprising particles of the nucleic acid construct dispersed within the dried excipient material, usually in the presence of excess powdered excipient. The weight ratio of nucleic acid construct to hydrophilic excipient in the initial solution is preferably from 1:1 to 1:10, and the solution may be dried by spraying droplets into a flowing gas stream (spray drying) or by vacuum drying to produce a crude powder followed by grinding to produce a final powder.

In the case of particles intended for lung delivery, having a particle size from 0.5 μm to 5 μm, each particle usually contains from 10³ to 10⁴ nucleic acid constructs. The constructs can be uniformly or non-uniformly dispersed in each particle, and the particles in turn will often be present in excess powdered excipient, usually at a weight ratio (nucleic acid construct:excipient powder free from nucleic acids) usually in the range from 1:10 to 1:500.

Methods for delivering nucleic acid constructs comprise directing the dry powder containing the nucleic acid constructs to a moist target location in a host, where the hydrophilic excipient matrix material of the particles will dissolve when exposed to the moist target location, leaving the much smaller nucleic acid construct particles to freely interact with cells. In a preferred embodiment, the target location is the lung and the particles are directed to the lung by inhalation.

Dry powder compositions are particularly advantageous since the hydrophilic excipient will stabilize the nucleic acid constructs for storage. Excess powdered hydrophilic excipient can also enhance dispersion of the dry powders into aerosols and, because of its high water solubility, facilitate dissolution of the composition to deposit the nucleic acid constructs into intimate contact with the target membranes, such as the lung surface membrane of the host.

Formulation of Viral Vectors

Measles virus vectors are purified by size exclusion chromatography using an Äkta FPLC system. The buffer used for chromatography is phosphate-buffer saline (PBS). The recombinant viruses can then directly be used to infect animals by different routes, for example, intranasally or orally.

Formulation of Prokaryotic Vectors

Bacteria are grown in selective media until reaching a growth plateau (e.g. 24 h in several 1 L shake flasks after a 1:500 inoculation). The cells are pelleted by centrifugation and the supernatant is discarded. The cell pellet is washed five times with 3 liters of PBS and then resuspended in 200 ml. The solution is ready for in vivo transduction via various routes (for example, intranasally or orally).

### Example 13

### Immunization Protocols

This Example describes immunization protocols that are used throughout the subsequent Examples. Mice are immunized with plasmids, virus vectors or bacterial vectors expressing full-length SARS-CoV M, E and S structural proteins. Alternatively, mice are immunized with SARS-CoV-VLPs isolated from cell culture medium. Immunization of mice is either orally, intravenously, or intraperitoneally with up to 100 μg of plasmids up to three times every two weeks. In the case of viral vectors, approximately 10⁷ to 10¹⁰ infectious virus are administered up to three times every two weeks. Approximately 10⁵ to 10⁶ prokaryotic delivery vectors, which comprise one or more of the SARS-CoV polypeptide-expressing constructs described herein, are administered up to three times every two weeks. For SARS-CoV-VLPs, concentrations of between about 1 μg and about 10 mg are administered up to three time every two weeks.

Cell-mediated immune responses to immunization are assayed by one or more of the following assays:

Lymphocyte Proliferation Assay

Spleenocytes are pulsed with Con A or non-stimulated, to measure mitogen-driven proliferation by ³H-thymidine incorporation. To measure antigen-specific proliferation, lymphocytes are cultured as above, but pulsed with 0.1 to 10 μg/ml of SARS-CoV recombinant proteins, or partially purified SARS-CoV-VLPs, with or without IL-2, and harvested after 7 days. Spontaneous proliferation is assayed in cultures without any antigen.

Lymphokine Responses

To determine production of stimulatory lymphokines, spleenocytes (5-10×10⁵) are incubated with or without specific antigens or Con A in triplicate cultures. Culture medium is RPMI containing 1% normal mouse serum and antibiotic. After 48 hours of incubation at 37° C., 100 μl aliquots of medium is removed and frozen. The content of lymphokine in the culture medium is assayed with HT2 cells that respond to lymphokine stimulation.

ELISpot

Populations of CD4⁺-CD8⁻ cells are purified using a cell sorter. ELISpot assays are performed to assess the contributions of each T-cell compartment. Assays use to detect SARS-CoV cellular responses include T cell proliferation, IL-4 and IFN-gamma ELISPOT. Methods of for such assays are well known in the art.

CTLs

CTL assays are performed using spleenocytes. CTL activity is measured by a conventional ⁵¹chromium-release assay. Secondary CTL responses are measured by re-stimulation in vitro as previously described. EL4 and EG7 cells transfected with plasmids that express SARS antigens are used as targets. To determine the CTL precursor levels we use specific tetrameric MHC class 1 molecules or ELIspot. This technique permits the direct quantification of the number of T-cell precursors produced by immunization with SARS-CoV immunogenic preparations described herein.

SARS Proteins as Tumor Rejection Antigen

The rejection of tumors expressing viral antigens is primarily mediated by cell-mediated immune responses. The cell-mediated immune response against tumors expressing virally encoded tumor antigens likely involves both CD4⁺ and CD8⁺ T cells.

The SARS-CoV immunogenic preparations described herein are used to induce protective tumor immunity in mice. Mice are immunized intraperitoneally with plasmids, virus vectors or bacterial vectors expressing SARS M, E or S antigens. Seven days after last inoculation, animals are challenged mid-flank bilaterally with 1×10⁵ of B16 expressing the corresponding SARS antigen, 10 times the dose lethal to 50% of the animals (LD₅₀). B16, EL4 and EG7 cell lines are constructed by infecting B16 with murine retrovirus vectors expressing SARS-CoV antigens. Local tumor growth and mouse survival is determined. In a parallel experiment, it is determined whether levels of CTL activities correlate with tumor rejection. Additionally, to correlate tumor rejection activities with an immunization regime, the dependence of tumor rejection activity on the number of doses and the size of inoculations is determined.

### Example 14

### Administration of SARS-CoV Immunogens

Preparations of isolated SARS-CoV-VLPs or preparations of genetic constructs described in the previous Examples, which are capable of producing SARS-CoV-VLPs, are inoculated into C57blk/6 mice. The preparations can be inoculated orally, intravenously or intraperitoneally. Induction of specific antibodies in mice is analyzed by enzyme-linked immunosorbent assay (ELISA). Sera from inoculated animals is analyzed every two weeks for 10 weeks to determine the evolution of the antibody response.

In other experiments, animals are inoculated with increasing amounts of preparations of isolated SARS-CoV-VLPs or preparations of genetic constructs described in the previous Examples, which are capable of producing SARS-CoV-VLPs, to establish whether the size of the inoculum can determine the level of specific antibody production. Furthermore, to determine preferred infection regimes for immunization, mice are inoculated intraperitoneally on one, two, three or four occasions with a fixed amount of the immunogenic preparations. Titers of antibodies in serum that react with SARS virus structural proteins is determined using a standard ELISA assay.

The approach described herein permits development of a strategy that enables simultaneous vaccination against multiple antigenic determinants through preparation of recombinant vectors expressing different SARS-CoV antigenic proteins. To test this possibility mice are inoculated with plasmids expressing individual as well as multiple SARS-CoV polypeptide. The titer of antibodies recognizing each of the SARS virus proteins can be determined by ELISA. This approach is useful in determining whether individually expressed SARS-CoV antigens can induce a protective immune response against the SARS virus. This approach can also be used to compare the efficacy of a single antigen approach with the efficacy of a multiple antigen approach. Regardless of which approach is determined superior, it will be appreciated that it is beneficial to induce any level of immune response against the SARS virus, and the induction of an immune response which is not fully protective is within the scope of the present invention.

### Example 15

### Induction of an Immune Response to SARS-CoV

It is well accepted that neutralizing antibodies are important components of an immune response that protect against pathogens that gain access to the subject through the mucosal surface. For example, it has been shown that immunity can be transferred by the delivery of neutralizing antibodies through the milk of lactating recombinant animals (Kolb et al., 2001).

The following experiments demonstrate that antibodies which are selected based on an in vitro assay are beneficial in animals. In these experiments, mice are inoculated with the vectors described in the above Examples which enable the formation of VLPs. In particular, mice are inoculated several times to induce high titers of antibodies and bled after 4 to 6 weeks. Sera from vaccinated animals is used to carry out SARS-CoV neutralizing assays.

Anti-SARS-CoV antibodies that do not neutralize the virus in tissue culture can have a beneficial effect in vivo, particularly at a mucosal surface. For example, antibodies that bind to native virions can facilitate virus clearance, virus destruction mediated by complement, inhibit transport or transcytosis to target tissues, or simply reduce the mobility of the virus through the mucus layers (Robert-Guroff, 2000). Each of these effects reduce the ability of the virus to cause SARS.

Mice are incoculated with recombinant vectors which express SARS-CoV structural proteins so as to induce high titers of antibodies capable of binding to intact SARS-CoV virions. In one experiment, sera from the immunized mice is collected an used to immunoprecipitate SARS-CoV-VLPs. Western blots are performed to detect SARS-CoV structural proteins in the precipitated virion fraction. Particular SARS-CoV antigen combinations which are identified as inducing antibodies that bind to native virions are further examined for disease protection in nonhuman primate models.

Cytotoxic T Lymphocytes (CTLs) kill neoplastic or virally infected cells after recognizing on their surface antigenic peptides bound to the major histocompatibility complex class I molecule. Immunizations with killed pathogens or their proteins normally do not generally elicit CTLs.

The role of CTL in the protective immune response against viruses is not completely understood. However, because CTLs are important in eliminating a wide variety of intracellular pathogens, including other coronaviruses, stimulating CTL production is beneficial in the induction of an immune response against SARS-CoV. Furthermore, in the protection against a respiratory disease, CTLs play a significant role in the protection of the respiratory mucosa against viruses. Protection can also be conferred to naive animals by transfer of CTL from vaccinated animals.

The following experiments demonstrate the ability of the immunogenic SARS-CoV preparations described herein to elicit an effective CTL response. After inoculation of mice and macaques with the immunogenic SARS-CoV preparations, high levels of CTL activity as well as high levels of CTL precursors are shown. In addition, it is demonstrated that immunization protects mice from subsequent challenge with tumors transfected with SARS antigens.

The methods, compositions, and systems described herein are presently representative of preferred embodiments and are exemplary and are not intended as limitations on the scope of the invention. Changes therein and other uses will occur to those skilled in the art which are encompassed within the spirit of the invention and are defined by the scope of the disclosure. Accordingly, it will be apparent to one skilled in the art that varying substitutions and modifications may be made to the invention disclosed herein without departing from the scope and spirit of the invention.

The terms “comprise,” “comprises,” and “comprising” as used in the claims below and throughout this specification do not limit the claimed invention to exclude any variants or additions. Rather, the terms “comprise,” “comprises,” and “comprising” mean “including, but not necessarily limited to.” For example, a method, apparatus, molecule or other item which contains A, B, and C may be accurately said to comprise A and B. Likewise, a method, apparatus, molecule or other item which “comprises A and B” may include any number of additional steps, components, atoms or other items as well.

As used in the claims below and throughout this disclosure, by the phrase “consisting essentially of” is meant including any elements listed after the phrase, and limited to other elements that do not interfere with or significantly contribute to the activity or action specified in the disclosure for the listed elements. Thus, the phrase “consisting essentially of” indicates that the listed elements are required or mandatory, but that other elements are optional and may or may not be present depending upon whether or not they significantly affect the activity or action of the listed elements.

The citation of references in this specification does not imply that any of these references are prior art to the present invention or that the content of any of these references constitutes common or general knowledge to those of ordinary skill in the art.

The disclosures of each of the references cited herein, including the following references, are incorporated herein by reference in their entireties.

### REFERENCES

Armstrong et al., 1984, Nature, 308, 751-752
Cavallaro and Monto, 1970, J. Infect. Dis., 122, 272-279
Cavanagh et al., 1984, Avian Pathology 13, 573-583
Cavanagh, 1995, In S. G. Siddell, The coronoviridae. Plenum Press, New York, London
Christianson et al., 1989, Arch. Virol., 109, 185-196
Delmas and Laude, 1990, J. Virol., 64, 5367-5375
Drosten et al., 2003, N. Engl. J. Med., online release
Eljamal et al., 1995, Assignee: Inhale Therapeutics, patent application Ser. No. 422,563
FDA, 1996 FDA CBER Website.
Godet et al., 1992, Virology, 188, 666-675
Grosse et al. 1994, Virology, 202, 814-824
Hamre and Prochnow, 1966, Proc. Soc. Exp. Biol. Med., 121, 190-193
Hofmann and Wyler, 1987, Schweiz. Arch. Tierheilk., 129, 437-442
Holmes et al., 1981, Virology, 115, 334-344
Kolb et al., 2001, J Virol., 75, 2803-2809
Kozak, 1987, Nucleic Acids Res., 15, 8125-48.
Ksiazek et al., 2003, N. Engl. J. Med., 348, 1947
Lee et al., 2003, N. Engl. J. Med., 348, 1986-94.
Liu and Inglis, 1991, Virology 185, 911-917
Luytyes et al., 1987, Virology, 161, 479-487
MacNaughton at al., 1978, J. Gen. Virol., 39, 545-549
MacNaughton et al, 1983, Infect. Immun., 38, 419-423
Matsumoto and Kawana, 1992, Kansenshogaku Zasshi, 66, 319-326
Niemann and Klenk, 1981, J. Mol. Biol., 153, 993-1010
Opstelten et al., 1993, J. Virol., 67, 7394-7401
Opstelten et al., 1994, Adv. Exp. Med. Biol., 342, 189-195
Opstelten et al., 1995, Adv. Exp. Med. Biol., 380, 291-297
Peiris et al., 2003, Lancet, 361, 1319
Poutanen et al., 2003, N. Engl. J. Med., 348, 1995-2005
Risco et al, 1996, J. Virol., 70, 4773-4777
Robert-Guroff, 2000, Nat Med., 6, 129-130.
Rottier at al., 1981, J. Virol., 40, 350-357
Rottier at al., 1986, Biochemistry, 25, 1335-1339
Sambrook et al., 2001, A laboratory manual
Seo et al., 2000, Virology, 269, 183-189
Sturman et al., 1980, J. Virol., 33, 449-462
Takamura et al., 2002, Can J Vet Res., 66, 278-281.
Takeda et al., 2000, J Virol., 74, 6643-6647
Tooze et al., 1987, J. Cell. Biol., 105, 1215-1226
Tsang et al., 2003, N. Engl. J. Med., 348, 1977-1985
Tyrell et al., 1965, Br. Med. J., 1, 1467-1470
Venemma et al., 1990, J. Virol., 64, 330-346
Vennema et al., 1991, Virology, 181, 327-335
Venemma et al., 1996, EMBO J., 15, 2020-2028
Zhang et al., 1994, J Med Virol. 44, 152-161
Marra et al., 2003, Science, 300, 1399-1405
Rota et al., 2003, Science, 300, 1394-1399

1. A system for making SARS-CoV virus-like particles (SARS-CoV-VLPs) comprising one or more recombinant vectors which express the SARS-CoV E-protein, the SARS-CoV M-protein and the SARS-CoV S-protein.

2. The system of claim 1, wherein said SARS-CoV E-protein, said SARS-CoV M-protein and said SARS-CoV S-protein are expressed from a single recombinant vector.

3. The system of claim 1, wherein said SARS-CoV E-protein, said SARS-CoV M-protein, and said SARS-CoV S-protein are expressed from a plurality of recombinant vectors.

4. The system of claim 1, wherein said one or more recombinant vectors comprise a plasmid.

5. The system of claim 1, wherein said one or more recombinant vectors comprise a recombinant virus.

6. The system of claim 5, wherein said recombinant virus is a measles virus.

7. A cell which has been engineered to express the SARS-CoV E-protein, the SARS-CoV M-protein, and the SARS-CoV S-protein.

8. The cell of claim 7, wherein said cell is live.

9. The cell of claim 8, wherein said cell is a bacterial cell.

10. The cell of claim 9, wherein said cell is a bacterial cell whose pathogenicity has been attenuated.

11. The cell of claim 10, wherein said cell is a Salmonella cell.

12. A method of inducing an immune response comprising administering to a subject one or more recombinant vectors which express the SARS-CoV E-protein, the SARS-CoV M-protein and the SARS-CoV S-protein.

13. The method of claim 12, wherein said SARS-CoV E-protein, the SARS-CoV M-protein and the SARS-CoV S-protein are expressed from a single recombinant vector.

14. The method of claim 12, wherein said SARS-CoV E-protein, the SARS-CoV M-protein and the SARS-CoV S-protein are expressed from a plurality of recombinant vectors.

15. The method of claim 12, wherein said one or more recombinant vectors comprise a plasmid.

16. The method of claim 12, wherein said one or more recombinant vectors comprise a virus.

17. The method of claim 12, wherein said one or more recombinant vectors comprise a prokaryotic vector.

18. The method of claim 12, wherein said subject is a human.

19. The method of claim 18, wherein said immune response is a cellular immune response.

20. The method of claim 18, wherein said immune response is a humoral immune response.

21. The method of claim 18, wherein said immune response is both a humoral and a cellular immune response.

22. A method of inducing an immune response in a subject comprising administering SARS-CoV-VLPs to said subject.

23. A method of inducing an immune response in a subject comprising administering a nucleic acid encoding the SARS-CoV E-protein, the SARS-CoV M-protein, and the SARS-CoV S-protein to said subject.

24. A SARS-CoV-VLP.

25. An isolated SARS-CoV-VLP of claim 24.


SEQUENCE LISTING (49 sequences)

SEQ ID NO: 1 - RNA, 8 nt, SARS Coronavirus
  misc_RNA (0)...(0): Transcription Associated Sequence (TAS)
ucuaaacu

SEQ ID NO: 2 - protein, 76 aa, SARS Coronavirus
MYSFVSEETGTLIVNSVLLFLAFVVFLLVTLAILTALRLCAYCCNIVNVSLVKPTVYVYSRVKNLNSSEGVPDLLV

SEQ ID NO: 3 - protein, 76 aa, SARS Coronavirus; identical to SEQ ID NO: 2

SEQ ID NO: 4 - protein, 76 aa, SARS Coronavirus; identical to SEQ ID NO: 2

SEQ ID NO: 5 - protein, 76 aa, SARS Coronavirus; identical to SEQ ID NO: 2

SEQ ID NO: 6 - protein, 221 aa, SARS Coronavirus
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 7 - protein, 221 aa, SARS Coronavirus
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHPLGRCDIKDLPKEITVATSRTLSYYKLGASQRVGTDSGFAAYNRYRIGNYKLNTDHAGSNDNIALLVQ

SEQ ID NO: 8 - protein, 221 aa, SARS Coronavirus
MADNGTITVEELKQLLEQWNLVIGFLFLAWIMLLQFAYSNRNRFLYIIKLVFLWLLWPVTLACFVLAVVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 9 - protein, 221 aa, SARS Coronavirus; identical to SEQ ID NO: 6

SEQ ID NO: 10 - protein, 1255 aa, SARS Coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 11 - protein, 1255 aa, SARS Coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 12 - protein, 1255 aa, SARS Coronavirus; identical to SEQ ID NO: 11

SEQ ID NO: 13 - protein, 1255 aa, SARS Coronavirus
MFIFLLFLTLTSGSDLDRCTTFDDVQAPNYTQHTSSMRGVYYPDEIFRSDTLYLTQDLFLPFYSNVTGFHTINHTFDNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDTWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 14 - DNA, 29751 bp, SARS Coronavirus
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgtgagctcaatggaggtgcagtcactcgctatgtcgacaacaatttctgtggcccagatgggtaccctcttgattgcatcaaagattttctcgcacgcgcgggcaagtcaatgtgcactctttccgaacaacttgattacatcgagtcgaagagaggtgtctactgctgccgtgaccatgagcatgaaattgcctggttcactgagcgctctgataagagctacgagcaccagacacccttcgaaattaagagtgccaagaaatttgacactttcaaaggggaatgcccaaagtttgtgtttcctcttaactcaaaagtcaaagtcattcaaccacgtgttgaaaagaaaaagactgagggtttcatggggcgtatacgctctgtgtaccctgttgcatctccacaggagtgtaacaatatgcacttgtctaccttgatgaaatgtaatcattgcgatgaagtttcatggcagacgtgcgactttctgaaagccacttgtgaacattgtggcactgaaaatttagttattgaaggacctactacatgtgggtacctacctactaatgctgtagtgaaaatgccatgtcctgcctgtcaagacccagagattggacctgagcatagtgttgcagattatcacaaccactcaaacattgaaactcgactccgcaagggaggtaggactagatgttttggaggctgtgtgtttgcctatgttggctgctataataagcgtgcctactgggttcctcgtgctagtgctgatattggctcaggccatactggcattactggtgacaatgtggagaccttgaatgaggatctccttgagatactgagtcgtgaacgtgttaacattaacattgttggcgattttcatttgaatgaagaggttgccatcattttggcatctttctctgcttctacaagtgcctttattgacactataaagagtcttgattacaagtctttcaaaaccattgttgagtcctgcggtaactataaagttaccaagggaaagcccgtaaaaggtgcttggaacattggacaacagagatcagttttaacaccactgtgtggttttccctcacaggctgctggtgttatcagatcaatttttgcgcgcacacttgatgcagcaaaccactcaattcctgatttgcaaagagcagctgtcaccatacttgatggtatttctgaacagtcattacgtcttgtcgacgccatggtttatacttcagacctgctcaccaacagtgtcattattatggcatatgtaactggtggtcttgtacaacagacttctcagtggttgtctaatcttttgggcactactgttgaaaaactcaggcctatctttgaatggattgaggcgaaacttagtgcaggagttgaatttctcaaggatgcttgggagattctcaaatttctcattacaggtgtttttgacatcgtcaagggtcaaatacaggttgcttcagataacatcaaggattgtgtaaaatgcttcattgatgttgttaacaaggcactcgaaatgtgcattgatcaagtcactatcgctggcgcaaagttgcgatcactcaacttaggtgaagtcttcatcgctcaaagcaagggactttaccgtcagtgtatacgtggcaaggagcagctgcaactactcatgcctcttaaggcaccaaaagaagtaacctttcttgaaggtgattcacatgacacagtacttacctctgaggaggttgttctcaagaacggtgaactcgaagcactcgagacgcccgttgatagcttcacaaatggagctatcgttggcacaccagtctgtgtaaatggcctcatgctcttagagattaaggacaaagaacaatactgcgcattgtctcctggtttactggctacaaacaatgtctttcgcttaaaagggggtgcaccaattaaaggtgtaacctttggagaagatactgtttgggaagttcaaggttacaagaatgtgagaatcacatttgagcttgatgaacgtgttgacaaagtgcttaatgaaaagtgctctgtctacactgttgaatccggtaccgaagttactgagtttgcatgtgttgtagcagaggctgttgtgaagactttacaaccagtttctgatctccttaccaacatgggtattgatcttgatgagtggagtgtagctacattctacttatttgatgatgctggtgaagaaaacttttcatcacgtatgtattgttccttttaccctccagatgaggaagaagaggacgatgcagagtgtgaggaagaagaaattgatgaaacctgtgaacatgagtacggtacagaggatgattatcaaggtctccctctggaatttggtgcctcagctgaaacagttcgagttgaggaagaagaagaggaagactggctggatgatactactgagcaatcagagattgagccagaaccagaacctacacctgaagaaccagttaatcagtttactggttatttaaaacttactgacaatgttgccattaaatgtgttgacatcgttaaggaggcacaaagtgctaatcctatggtgattgtaaatgctgctaacatacacctgaaacatggtggtggtgtagcaggtgcactcaacaaggcaaccaatggtgccatgcaaaaggagagtgatgattacattaagctaaatggccctcttacagtaggagggtcttgtttgctttctggacataatcttgctaagaagtgtctgcatgttgttggacctaacctaaatgcaggtgaggacatccagcttcttaaggcagcatatgaaaatttcaattcacaggacatcttacttgcaccattgttgtcagcaggcatatttggtgctaaaccacttcagtctttacaagtgtgcgtgcagacggttcgtacacaggtttatattgcagtcaatgacaaagctctttatgagcaggttgtcatggattatcttgataacctgaagcctagagtggaagcacctaaacaagaggagccaccaaacacagaagattccaaaactgaggagaaatctgtcgtacagaagcctgtcgatgtgaagccaaaaattaaggcctgcattgatgaggttaccacaacactggaagaaactaagtttcttaccaataagttactcttgtttgctgatatcaatggtaagctttaccatgattctcagaacatgcttagaggtgaagatatgtctttccttgagaaggatgcaccttacatggtaggtgatgttatcactagtggtgatatcacttgtgttgtaataccctccaaaaaggctggtggcactactgagatgctctcaagagctttgaagaaagtgccagttgatgagtatataaccacgtaccctggacaaggatgtgctggttatacacttgaggaagctaagactgctcttaagaaatgcaaatctgcattttatgtactaccttcagaagcacctaatgctaaggaagagattctaggaactgtatcctggaatttgagagaaatgcttgctcatgctgaagagacaagaaaattaatgcctatatgcatggatgttagagccataatggcaaccatccaacgtaagtataaaggaattaaaattcaagagggcatcgttgactatggtgtccgattcttcttttatactagtaaagagcctgtagcttctattattacgaagctgaactctctaaatgagccgcttgtcacaatgccaattggttatgtgacacatggttttaatcttgaagaggctgcgcgctgtatgcgttctcttaaagctcctgccgtagtgtcagtatcatcaccagatgctgttactacatataatggatacctcacttcgtcatcaaagacatctgaggagcactttgtagaaacagtttctttggctggctcttacagagattggtcctattcaggacagcgtacagagttaggtgttgaatttcttaagcgtggtgacaaaattgtgtaccacactctggagagccccgtcgagtttcatcttgacggtgaggttctttcacttgacaaactaaagagtctcttatccctgcgggaggttaagactataaaagtgttcacaactgtggacaacactaatctccacacacagcttgtggatatgtctatgacatatggacagcagtttggtccaacatacttggatggtgctgatgttacaaaaattaaacctcatgtaaatcatgagggtaagactttctttgtactacctagtgatgacacactacgtagtgaagctttcgagtactaccatactcttgatgagagttttcttggtaggtacatgtctgctttaaaccacacaaagaaatggaaatttcctcaagttggtggtttaacttcaattaaatgggctgataacaattgttatttgtctagtgttttattagcacttcaacagcttgaagtcaaattcaatgcaccagcacttcaagaggcttattatagagcccgtgctggtgatgctgctaacttttgtgcactcatactcgcttacagtaataaaactgttggcgagcttggtgatgtcagagaaactatgacccatcttctacagcatgctaatttggaatctgcaaagcgagttcttaatgtggtgtgtaaacattgtggtcagaaaactactaccttaacgggtgtagaagctgtgatgtatatgggtactctatcttatgataatcttaagacaggtgtttccattccatgtgtgtgtggtcgtgatgctacacaatatctagtacaacaagagtcttcttttgttatgatgtctgcaccacctgctgagtataaattacagcaaggtacattcttatgtgcgaatgagtacactggtaactatcagtgtggtcattacactcatataactgctaaggagaccctctatcgtattgacggagctcaccttacaaagatgtcagagtacaaaggaccagtgactgatgttttctacaaggaaacatcttacactacaaccatcaagcctgtgtcgtataaactcgatggagttacttacacagagattgaaccaaaattggatgggtattataaaaaggataatgcttactatacagagcagcctatagaccttgtaccaactcaaccattaccaaatgcgagttttgataatttcaaactcacatgttctaacacaaaatttgctgatgatttaaatcaaatgacaggcttcacaaagccagcttcacgagagctatctgtcacattcttcccagacttgaatggcgatgtagtggctattgactatagacactattcagcgagtttcaagaaaggtgctaaattactgcataagccaattgtttggcacattaaccaggctacaaccaagacaacgttcaaaccaaacacttggtgtttacgttgtctttggagtacaaagccagtagatacttcaaattcatttgaagttctggcagtagaagacacacaaggaatggacaatcttgcttgtgaaagtcaacaacccacctctgaagaagtagtggaaaatcctaccatacagaaggaagtcatagagtgtgacgtgaaaactaccgaagttgtaggcaatgtcatacttaaaccatcagatgaaggtgttaaagtaacacaagagttaggtcatgaggatcttatggctgcttatgtggaaaacacaagcattaccattaagaaacctaatgagctttcactagccttaggtttaaaaacaattgccactcatggtattgctgcaattaatagtgttccttggagtaaaattttggcttatgtcaaaccattcttaggacaagcagcaattacaacatcaaattgcgctaagagattagcacaacgtgtgtttaacaattatatgccttatgtgtttacattattgttccaattgtgtacttttactaaaagtaccaattctagaattagagcttcactacctacaactattgctaaaaatagtgttaagagtgttgctaaattatgtttggatgccggcattaattatgtgaagtcacccaaattttctaaattgttcacaatcgctatgtggctattgttgttaagtatttgcttaggttctctaatctgtgtaactgctgcttttggtgtactcttatctaattttggtgctccttcttattgtaatggcgttagagaattgtatcttaattcgtctaacgttactactatggatttctgtgaaggttcttttccttgcagcatttgtttaagtggattagactcccttgattcttatccagctcttgaaaccattcaggtgacgatttcatcgtacaagctagacttgacaattttaggtctggccgctgagtgggttttggcatatatgttgttcacaaaattcttttatttattaggtctttcagctataatgcaggtgttctttggctattttgctagtcatttcatcagcaattcttggctcatgtggtttatcattagtattgtacaaatggcacccgtttctgcaatggttaggatgtacatcttctttgcttctttctactacatatggaagagctatgttcatatcatggatggttgcacctcttcgacttgcatgatgtgctataagcgcaatcgtgccacacgcgttgagtgtacaactattgttaatggcatgaagagatctttctatgtctatgcaaatggaggccgtggcttctgcaagactcacaattggaattgtctcaattgtgacacattttgcactggtagtacattcattagtgatgaagttgctcgtgatttgtcactccagtttaaaagaccaatcaaccctactgaccagtcatcgtatattgttgatagtgttgctgtgaaaaatggcgcgcttcacctctactttgacaaggctggtcaaaagacctatgagagacatccgctctcccattttgtcaatttagacaatttgagagctaacaacactaaaggttcactgcctattaatgtcatagtttttgatggcaagtccaaatgcgacgagtctgcttctaagtctgcttctgtgtactacagtcagctgatgtgccaacctattctgttgcttgaccaagctcttgtatcagacgttggagatagtactgaagtttccgttaagatgtttgatgcttatgtcgacaccttttcagcaacttttagtgttcctatggaaaaacttaaggcacttgttgctacagctcacagcgagttagcaaagggtgtagctttagatggtgtcctttctacattcgtgtcagctgcccgacaaggtgttgttgataccgatgttgacacaaaggatgttattgaatgtctcaaactttcacatcactctgacttagaagtgacaggtgacagttgtaacaatttcatgctcacctataataaggttgaaaacatgacgcccagagatcttggcgcatgtattgactgtaatgcaaggcatatcaatgcccaagtagcaaaaagtcacaatgtttcactcatctggaatgtaaaagactacatgtctttatctgaacagctgcgtaaacaaattcgtagtgctgccaagaagaacaacataccttttagactaacttgtgctacaactagacaggttgtcaatgtcataactactaaaatctcactcaagggtggtaagattgttagtacttgttttaaacttatgcttaaggccacattattgtgcgttcttgctgcattggtttgttatatcgttatgccagtacatacattgtcaatccatgatggttacacaaatgaaatcattggttacaaagccattcaggatggtgtcactcgtgacatcatttctactgatgattgttttgcaaataaacatgctggttttgacgcatggtttagccagcgtggtggttcatacaaaaatgacaaaagctgccctgtagtagctgctatcattacaagagagattggtttcatagtgcctggcttaccgggtactgtgctgagagcaatcaatggtgacttcttgcattttctacctcgtgtttttagtgctgttggcaacatttgctacacaccttccaaactcattgagtatagtgattttgctacctctgcttgcgttcttgctgctgagtgtacaatttttaaggatgctatgggcaaacctgtgccatattgttatgacactaatttgctagagggttctatttcttatagtgagcttcgtccagacactcgttatgtgcttatggatggttccatcatacagtttcctaacacttacctggagggttctgttagagtagtaacaacttttgatgctgagtactgtagacatggtacatgcgaaaggtcagaagtaggtatttgcctatctaccagtggtagatgggttcttaataatgagcattacagagctctatcaggagttttctgtggtgttgatgcgatgaatctcatagctaacatctttactcctcttgtgcaacctgtgggtgctttagatgtgtctgcttcagtagtggctggtggtattattgccatattggtgacttgtgctgcctactactttatgaaattcagacgtgtttttggtgagtacaaccatgttgttgctgctaatgcacttttgtttttgatgtctttcactatactctgtctggtaccagcttacagctttctgccgggagtctactcagtcttttacttgtacttgacattctatttcaccaatgatgtttcattcttggctcaccttcaatggtttgccatgttttctcctattgtgcctttttggataacagcaatctatgtattctgtatttctctgaagcactgccattggttctttaacaactatcttaggaaaagagtcatgtttaatggagttacatttagtaccttcgaggaggctgctttgtgtacctttttgctcaacaaggaaatgtacctaaaattgcgtagcgagacactgttgccacttacacagtataacaggtatcttgctctatataacaagtacaagtatttcagtggagccttagatactaccagctatcgtgaagcagcttgctgccacttagcaaaggctctaaatgactttagcaactcaggtgctgatgttctctaccaaccaccacagacatcaatcacttctgctgttctgcagagtggttttaggaaaatggcattcccgtcaggcaaagttgaagggtgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcagttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataacattaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaaggtaagttcaagaaaattgttaagggcactcatcattggatgcttttaactttcttgacatcactattgattcttgttcaaagtacacagtggtcactgtttttctttgtttacgagaatgctttcttgccatttactcttggtattatggcaattgctgcatgtgctatgctgcttgttaagcataagcacgcattcttgtgcttgtttctgttaccttctcttgcaacagttgcttactttaatatggtctacatgcctgctagctgggtgatgcgtatcatgacatggcttgaattggctgacactagcttgtctggttataggcttaaggattgtgttatgtatgcttcagctttagttttgcttattctcatgacagctcgcactgtttatgatgatgctgctagacgtgtttggacactgatgaatgtcattacacttgtttacaaagtctactatggtaatgctttagatcaagctatttccatgtgggccttagttatttctgtaacctctaactattctggtgtcgttacgactatcatgtttttagctagagctatagtgtttgtgtgtgttgagtattacccattgttatttattactggcaacaccttacagtgtatcatgcttgtttattgtttcttaggctattgttgctgctgctactttggccttttctgtttactcaaccgttacttcaggcttactcttggtgtttatgactacttggtctctacacaagaatttaggtatatgaactcccaggggcttttgcctcctaagagtagtattgatgctttcaagcttaacattaagttgttgggtattggaggtaaaccatgtatcaaggttgctactgtacagtctaaaatgtctgacgtaaagtgcacatctgtggtactgctctcggttcttcaacaacttagagtagagtcatcttctaaattgtgggcacaatgtgtacaactccacaatgatattcttcttgcaaaagacacaactgaagctttcgagaagatggtttctcttttgtctgttttgctatccatgcagggtgctgtagacattaataggttgtgcgaggaaatgctcgataaccgtgctactcttcaggctattgcttcagaatttagttctttaccatcatatgccgcttatgccactgcccaggaggcctatgagcaggctgtagctaatggtgattctgaagtcgttctcaaaaagttaaagaaatctttgaatgtggctaaatctgagtttgaccgtgatgctgccatgcaacgcaagttggaaaagatggcagatcaggctatgacccaaatgtacaaacaggcaagatctgaggacaagagggcaaaagtaactagtgctatgcaaacaatgctcttcactatgcttaggaagcttgataatgatgcacttaacaacattatcaacaatgcgcgtgatggttgtgttccactcaacatcataccattgactacagcagccaaactcatggttgttgtccctgattatggtacctacaagaacacttgtgatggtaacacctttacatatgcatctgcactctgggaaatccagcaagttgttgatgcggatagcaagattgttcaacttagtgaaattaacatggacaattcaccaaatttggcttggcctcttattgttacagctctaagagccaactcagctgttaaactacagaataatgaactgagtccagtagcactacgacagatgtcctgtgcggctggtaccacacaaacagcttgtactgatgacaatgcacttgcctactataacaattcgaagggaggtaggtttgtgctggcattactatcagaccaccaagatctcaaatgggctagattccctaagagtgatggtacaggtacaatttacacagaactggaaccaccttgtaggtttgttacagacacaccaaaagggcctaaagtgaaatacttgtacttcatcaaaggcttaaacaacctaaatagaggtatggtgctgggcagtttagctgctacagtacgtcttcaggctggaaatgctacagaagtacctgccaattcaactgtgctttccttctgtgcttttgcagtagaccctgctaaagcatataaggattacctagcaagtggaggacaaccaatcaccaactgtgtgaagatgttgtgtacacacactggtacaggacaggcaattactgtaacaccagaagctaacatggaccaagagtcctttggtggtgcttcatgttgtctgtattgtagatgccacattgaccatccaaatcctaaaggattctgtgacttgaaaggtaagtacgtccaaatacctaccacttgtgctaatgacccagtgggttttacacttagaaacacagtctgtaccgtctgcggaatgtggaaaggttatggctgtagttgtgaccaactccgcgaacccttgatgcagtctgcggatgcatcaacgtttttaaacgggtttgcggtgtaagtgcagcccgtcttacaccgtgcggcacaggcactagtactgatgtcgtctacagggcttttgatatttacaacgaaaaagttgctggttttgcaaagttcctaaaaactaattgctgtcgcttccaggagaaggatgaggaaggcaatttattagactcttactttgtagttaagaggcatactatgtctaactaccaacatgaagagactatttataacttggttaaagattgtccagcggttgctgtccatgactttttcaagtttagagtagatggtgacatggtaccacatatatcacgtcagcgtctaactaaatacacaatggctgatttagtctatgctctacgtcattttgatgagggtaattgtgatacattaaaagaaatactcgtcacatacaattgctgtgatgatgattatttcaataagaaggattggtatgacttcgtagagaatcctgacatcttacgcgtatatgctaacttaggtgagcgtgtacgccaatcattattaaagactgtacaattctgcgatgctatgcgtgatgcaggcattgtaggcgtactgacattagataatcaggatcttaatgggaactggtacgatttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatttgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacctgccttacccagatccatcaagaatattaggcgcaggctgttttgtcgatgatattgtcaaaacagatggtacacttatgattgaaaggttcgtgtcactggctattgatgcttacccacttacaaaacatcctaatcaggagtatgctgatgtctttcacttgtatttacaatacattagaaagttacatgatgagcttactggccacatgttggacatgtattccgtaatgctaactaatgataacacctcacggtactgggaacctgagttttatgaggctatgtacacaccacatacagtcttgcaggctgtaggtgcttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgccactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgccacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcattaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggataagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgcttcaatgtggctatcacaagggcaaaaattggcattttgtgcataatgtctgatagagatctttatgacaaactgcaatttacaagtctagaaataccacgtcgcaatgtggctacattacaagcagaaaatgtaactggactttttaaggactgtagtaagatcattactggtcttcatcctacacaggcacctacacacctcagcgttgatataaagttcaagactgaaggattatgtgttgacataccaggcataccaaaggacatgacctaccgtagactcatctctatgatgggtttcaaaatgaattaccaagtcaatggttaccctaatatgtttatcacccgcgaagaagctattcgtcacgttcgtgcgtggattggctttgatgtagagggctgtcatgcaactagagatgctgtgggtactaacctacctctccagctaggattttctacaggtgttaacttagtagctgtaccgactggttatgttgacactgaaaataacacagaattcaccagagttaatgcaaaacctccaccaggtgaccagtttaaacatcttataccactcatgtataaaggcttgccctggaatgtagtgcgtattaagatagtacaaatgctcagtgatacactgaaaggattgtcagacagagtcgtgttcgtcctttgggcgcatggctttgagcttacatcaatgaagtactttgtcaagattggacctgaaagaacgtgttgtctgtgtgacaaacgtgcaacttgcttttctacttcatcagatacttatgcctgctggaatcattctgtgggttttgactatgtctataacccatttatgattgatgttcagcagtggggctttacgggtaaccttcagagtaaccatgaccaacattgccaggtacatggaaatgcacatgtggctagttgtgatgctatcatgactagatgtttagcagtccatgagtgctttgttaagcgcgttgattggtctgttgaataccctattataggagatgaactgagggttaattctgcttgcagaaaagtacaacacatggttgtgaagtctgcattgcttgctgataagtttccagttcttcatgacattggaaatccaaaggctatcaagtgtgtgcctcaggctgaagtagaatggaagttctacgatgctcagccatgtagtgacaaagcttacaaaatagaggaactcttctattcttatgctacacatcacgataaattcactgatggtgtttgtttgttttggaattgtaacgttgatcgttacccagccaatgcaattgtgtgtaggtttgacacaagagtcttgtcaaacttgaacttaccaggctgtgatggtggtagtttgtatgtgaataagcatgcattccacactccagctttcgataaaagtgcatttactaatttaaagcaattgcctttcttttactattctgatagtccttgtgagtctcatggcaaacaagtagtgtcggatattgattatgttccactcaaatctgctacgtgtattacacgatgcaatttaggtggtgctgtttgcagacaccatgcaaatgagtaccgacagtacttggatgcatataatatgatgatttctgctggatttagcctatggatttacaaacaatttgatacttataacctgtggaatacatttaccaggttacagagtttagaaaatgtggcttataatgttgttaataaaggacactttgatggacacgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttcagtcatggacaacttggcggtcttcatttaatgataggcttagccaagcgctcacaagattcaccacttaaattagaggattttatccctatggacagcacagtgaaaaattacttcataacagatgcgcaaacaggttcatcaaaatgtgtgtgttctgtgattgatcttttacttgatgactttgtcgagataataaagtcacaagatttgtcagtgatttcaaaagtggtcaaggttacaattgactatgctgaaatttcattcatgctttggtgtaaggatggacatgttgaaaccttctacccaaaactacaagcaagtcaagcgtggcaaccaggtgttgcgatgcctaacttgtacaagatgcaaagaatgcttcttgaaaagtgtgaccttcagaattatggtgaaaatgctgttataccaaaaggaataatgatgaatgtcgcaaagtatactcaactgtgtcaatacttaaatacacttactttagctgtaccctacaacatgagagttattcactttggtgctggctctgataaaggagttgcaccaggtacagctgtgctcagacaatggttgccaactggcacactacttgtcgattcagatcttaatgacttcgtctccgacgcagattctactttaattggagactgtgcaacagtacatacggctaataaatgggaccttattattagcgatatgtatgaccctaggaccaaacatgtgacaaaagagaatgactctaaagaagggtttttcacttatctgtgtggatttataaagcaaaaactagccctgggtggttctatagctgtaaagataacagagcattcttggaatgctgacctttacaagcttatgggccatttctcatggtggacagcttttgttacaaatgtaaatgcatcatcatcggaagcatttttaattggggctaactatcttggcaagccgaaggaacaaattgatggctataccatgcatgctaactacattttctggaggaacacaaatcctatccagttgtcttcctattcactctttgacatgagcaaatttcctcttaaattaagaggaactgctgtaatgtctcttaaggagaatcaaatcaatgatatgatttattctcttctggaaaaaggtaggcttatcattagagaaaacaacagagttgtggtttcaagtgatattcttgttaacaactaaacgaacatgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgcgcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataaacgaacttatggatttgtttatgagattttttactcttagatcaattactgcacagccagtaaaaattgacaatgcttctcctgcaagtactgttcatgctacagcaacgataccgctacaagcctcactccctttcggatggcttgttattggcgttgcatttcttgctgtttttcagagcgctaccaaaataattgcgctcaataaaagatggcagctagccctttataagggcttccagttcatttgcaatttactgctgctatttgttaccatctattcacatcttttgcttgtcgctgcaggtatggaggcgcaatttttgtacctctatgccttgatatattttctacaatgcatcaacgcatgtagaattattatgagatgttggctttgttggaagtgcaaatccaagaacccattactttatgatgccaactactttgtttgctggcacacacataactatgactactgtataccatataacagtgtcacagatacaattgtcgttactgaaggtgacggcatttcaacaccaaaactcaaagaagactaccaaattggtggttattctgaggataggcactcaggtgttaaagactatgtcgttgtacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgctaatccagcaatggatccaatttatgatgagccgacgacgactactagcgtgcctttgtaagcacaagaaagtgagtacgaacttatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacactccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaagtgacaacagatgtttcatcttgttgacttccaggttacaatagcagagatattgattatcattatgaggactttcaggattgctatttggaatcttgacgttataataagttcaatagtgagacaattatttaagcctctaactaagaagaattattcggagttagatgatgaagaacctatggagttagattatccataaaacgaacatgaaaattattctcttcctgacattgattgtatttacatcttgcgagctatatcactatcaggagtgtgttagaggtacgactgtactactaaaagaaccttgcccatcaggaacatacgagggcaattcaccatttcaccctcttgctgacaataaatttgcactaacttgcactagcacacactttgcttttgcttgtgctgacggtactcgacatacctatcagctgcgtgcaagatcagtttcaccaaaacttttcatcagacaagaggaggttcaacaagagctctactcgccactttttctcattgttgctgctctagtatttttaatactttgcttcaccattaagagaaagacagaatgaatgagctcactttaattgacttctatttgtgctttttagcctttctgctattccttgttttaataatgcttattatattttggttttcactcgaaatccaggatctagaagaaccttgtaccaaagtctaaacgaacatgaaacttctcattgttttgacttgtatttctctatgcagttgcatatgcactgtagtacagcgctgtgcatctaataaacctcatgtgcttgaagatccttgtaaggtacaacactaggggtaatacttatagcactgcttggctttgtgctctaggaaaggttttaccttttcatagatggcacactatggttcaaacatgcacacctaatgttactatcaactgtcaagatccagctggtggtgcgcttatagctaggtgttggtaccttcatgaaggtcaccaaactgctgcatttagagacgtacttgttgttttaaataaacgaacaaattaaaatgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataaacactcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgacaaaaaaaaaaaaaaaaaaaaaaaa

SEQ ID NO: 15 - DNA, 29727 bp, SARS Coronavirus
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgtgagctcaatggaggtgcagtcactcgctatgtcgacaacaatttctgtggcccagatgggtaccctcttgattgcatcaaagattttctcgcacgcgcgggcaagtcaatgtgcactctttccgaacaacttgattacatcgagtcgaagagaggtgtctactgctgccgtgaccatgagcatgaaattgcctggttcactgagcgctctgataagagctacgagcaccagacacccttcgaaattaagagtgccaagaaatttgacactttcaaaggggaatgcccaaagtttgtgtttcctcttaactcaaaagtcaaagtcattcaaccacgtgttgaaaagaaaaagactgagggtttcatggggcgtatacgctctgtgtaccctgttgcatctccacaggagtgtaacaatatgcacttgtctaccttgatgaaatgtaatcattgcgatgaagtttcatggcagacgtgcgactttctgaaagccacttgtgaacattgtggcactgaaaatttagttattgaaggacctactacatgtgggtacctacctactaatgctgtagtgaaaatgccatgtcctgcctgtcaagacccagagattggacctgagcatagtgttgcagattatcacaaccactcaaacattgaaactcgactccgcaagggaggtaggactagatgttttggaggctgtgtgtttgcctatgttggctgctataataagcgtgcctactgggttcctcgtgctagtgctgatattggctcaggccatactggcattactggtgacaatgtggagaccttgaatgaggatctccttgagatactgagtcgtgaacgtgttaacattaacattgttggcgattttcatttgaatgaagaggttgccatcattttggcatctttctctgcttctacaagtgcctttattgacactataaagagtcttgattacaagtctttcaaaaccattgttgagtcctgcggtaactataaagttaccaagggaaagcccgtaaaaggtgcttggaacattggacaacagagatcagttttaacaccactgtgtggttttccctcacaggctgctggtgttatcagatcaatttttgcgcgcacacttgatgcagcaaaccactcaattcctgatttgcaaagagcagctgtcaccatacttgatggtatttctgaacagtcattacgtcttgtcgacgccatggtttatacttcagacctgctcaccaacagtgtcattattatggcatatgtaactggtggtcttgtacaacagacttctcagtggttgtctaatcttttgggcactactgttgaaaaactcaggcctatctttgaatggattgaggcgaaacttagtgcaggagttgaatttctcaaggatgcttgggagattctcaaatttctcattacaggtgtttttgacatcgtcaagggtcaaatacaggttgcttcagataacatcaaggattgtgtaaaatgcttcattgatgttgttaacaaggcactcgaaatgtgcattgatcaagtcactatcgctggcgcaaagttgcgatcactcaacttaggtgaagtcttcatcgctcaaagcaagggactttaccgtcagtgtatacgtggcaaggagcagctgcaactactcatgcctcttaaggcaccaaaagaagtaacctttcttgaaggtgattcacatgacacagtacttacctctgaggaggttgttctcaagaacggtgaactcgaagcactcgagacgcccgttgatagcttcacaaatggagctatcgttggcacaccagtctgtgtaaatggcctcatgctcttagagattaaggacaaagaacaatactgcgcattgtctcctggtttactggctacaaacaatgtctttcgcttaaaagggggtgcaccaattaaaggtgtaacctttggagaagatactgtttgggaagttcaaggttacaagaatgtgagaatcacatttgagcttgatgaacgtgttgacaaagtgcttaatgaaaagtgctctgtctacactgttgaatccggtaccgaagttactgagtttgcatgtgttgtagcagaggctgttgtgaagactttacaaccagtttctgatctccttaccaacatgggtattgatcttgatgagtggagtgtagctacattctacttatttgatgatgctggtgaagaaaacttttcatcacgtatgtattgttccttttaccctccagatgaggaagaagaggacgatgcagagtgtgaggaagaagaaattgatgaaacctgtgaacatgagtacggtacagaggatgattatcaaggtctccctctggaatttggtgcctcagctgaaacagttcgagttgaggaagaagaagaggaagactggctggatgatactactgagcaatcagagattgagccagaaccagaacctacacctgaagaaccagttaatcagtttactggttatttaaaacttactgacaatgttgccattaaatgtgttgacatcgttaaggaggcacaaagtgctaatcctatggtgattgtaaatgctgctaacatacacctgaaacatggtggtggtgtagcaggtgcactcaacaaggcaaccaatggtgccatgcaaaaggagagtgatgattacattaagctaaatggccctcttacagtaggagggtcttgtttgctttctggacataatcttgctaagaagtgtctgcatgttgttggacctaacctaaatgcaggtgaggacatccagcttcttaaggcagcatatgaaaatttcaattcacaggacatcttacttgcaccattgttgtcagcaggcatatttggtgctaaaccacttcagtctttacaagtgtgcgtgcagacggttcgtacacaggtttatattgcagtcaatgacaaagctctttatgagcaggttgtcatggattatcttgataacctgaagcctagagtggaagcacctaaacaagaggagccaccaaacacagaagattccaaaactgaggagaaatctgtcgtacagaagcctgtcgatgtgaagccaaaaattaaggcctgcattgatgaggttaccacaacactggaagaaactaagtttcttaccaataagttactcttgtttgctgatatcaatggtaagctttaccatgattctcagaacatgcttagaggtgaagatatgtctttccttgagaaggatgcaccttacatggtaggtgatgttatcactagtggtgatatcacttgtgttgtaataccctccaaaaaggctggtggcactactgagatgctctcaagagctttgaagaaagtgccagttgatgagtatataaccacgtaccctggacaaggatgtgctggttatacacttgaggaagctaagactgctcttaagaaatgcaaatctgcattttatgtactaccttcagaagcacctaatgctaaggaagagattctaggaactgtatcctggaatttgagagaaatgcttgctcatgctgaagagacaagaaaattaatgcctatatgcatggatgttagagccataatggcaaccatccaacgtaagtataaaggaattaaaattcaagagggcatcgttgactatggtgtccgattcttcttttatactagtaaagagcctgtagcttctattattacgaagctgaactctctaaatgagccgcttgtcacaatgccaattggttatgtgacacatggttttaatcttgaagaggctgcgcgctgtatgcgttctcttaaagctcctgccgtagtgtcagtatcatcaccagatgctgttactacatataatggatacctcacttcgtcatcaaagacatctgaggagcactttgtagaaacagtttctttggctggctcttacagagattggtcctattcaggacagcgtacagagttaggtgttgaatttcttaagcgtggtgacaaaattgtgtaccacactctggagagccccgtcgagtttcatcttgacggtgaggttctttcacttgacaaactaaagagtctcttatccctgcgggaggttaagactataaaagtgttcacaactgtggacaacactaatctccacacacagcttgtggatatgtctatgacatatggacagcagtttggtccaacatacttggatggtgctgatgttacaaaaattaaacctcatgtaaatcatgagggtaagactttctttgtactacctagtgatgacacactacgtagtgaagctttcgagtactaccatactcttgatgagagttttcttggtaggtacatgtctgctttaaaccacacaaagaaatggaaatttcctcaagttggtggtttaacttcaattaaatgggctgataacaattgttatttgtctagtgttttattagcacttcaacagcttgaagtcaaattcaatgcaccagcacttcaagaggcttattatagagcccgtgctggtgatgctgctaacttttgtgcactcatactcgcttacagtaataaaactgttggcgagcttggtgatgtcagagaaactatgacccatcttctacagcatgctaatttggaatctgcaaagcgagttcttaatgtggtgtgtaaacattgtggtcagaaaactactaccttaacgggtgtagaagctgtgatgtatatgggtactctatcttatgataatcttaagacaggtgtttccattccatgtgtgtgtggtcgtgatgctacacaatatctagtacaacaagagtcttcttttgttatgatgtctgcaccacctgctgagtataaattacagcaaggtacattcttatgtgcgaatgagtacactggtaactatcagtgtggtcattacactcatataactgctaaggagaccctctatcgtattgacggagctcaccttacaaagatgtcagagtacaaaggaccagtgactgatgttttctacaaggaaacatcttacactacaaccatcaagcctgtgtcgtataaactcgatggagttacttacacagagattgaaccaaaattggatgggtattataaaaaggataatgcttactatacagagcagcctatagaccttgtaccaactcaaccattaccaaatgcgagttttgataatttcaaactcacatgttctaacacaaaatttgctgatgatttaaatcaaatgacaggcttcacaaagccagcttcacgagagctatctgtcacattcttcccagacttgaatggcgatgtagtggctattgactatagacactattcagcgagtttcaagaaaggtgctaaattactgcataagccaattgtttggcacattaaccaggctacaaccaagacaacgttcaaaccaaacacttggtgtttacgttgtctttggagtacaaagccagtagatacttcaaattcatttgaagttctggcagtagaagacacacaaggaatggacaatcttgcttgtgaaagtcaacaacccacctctgaagaagtagtggaaaatcctaccatacagaaggaagtcatagagtgtgacgtgaaaactaccgaagttgtaggcaatgtcatacttaaaccatcagatgaaggtgttaaagtaacacaagagttaggtcatgaggatcttatggctgcttatgtggaaaacacaagcattaccattaagaaacctaatgagctttcactagccttaggtttaaaaacaattgccactcatggtattgctgcaattaatagtgttccttggagtaaaattttggcttatgtcaaaccattcttaggacaagcagcaattacaacatcaaattgcgctaagagattagcacaacgtgtgtttaacaattatatgccttatgtgtttacattattgttccaattgtgtacttttactaaaagtaccaattctagaattagagcttcactacctacaactattgctaaaaatagtgttaagagtgttgctaaattatgtttggatgccggcattaattatgtgaagtcacccaaattttctaaattgttcacaatcgctatgtggctattgttgttaagtatttgcttaggttctctaatctgtgtaactgctgcttttggtgtactcttatctaattttggtgctccttcttattgtaatggcgttagagaattgtatcttaattcgtctaacgttactactatggatttctgtgaaggttcttttccttgcagcatttgtttaagtggattagactcccttgattcttatccagctcttgaaaccattcaggtgacgatttcatcgtacaagctagacttgacaattttaggtctggccgctgagtgggttttggcatatatgttgttcacaaaattcttttatttattaggtctttcagctataatgcaggtgttctttggctattttgctagtcatttcatcagcaattcttggctcatgtggtttatcattagtattgtacaaatggcacccgtttctgcaatggttaggatgtacatcttctttgcttctttctactacatatggaagagctatgttcatatcatggatggttgcacctcttcgacttgcatgatgtgctataagcgcaatcgtgccacacgcgttgagtgtacaactattgttaatggcatgaagagatctttctatgtctatgcaaatggaggccgtggcttctgcaagactcacaattggaattgtctcaattgtgacacattttgcactggtagtacattcattagtgatgaagttgctcgtgatttgtcactccagtttaaaagaccaatcaaccctactgaccagtcatcgtatattgttgatagtgttgctgtgaaaaatggcgcgcttcacctctactttgacaaggctggtcaaaagacctatgagagacatccgctctcccattttgtcaatttagacaatttgagagctaacaacactaaaggttcactgcctattaatgtcatagtttttgatggcaagtccaaatgcgacgagtctgcttctaagtctgcttctgtgtactacagtcagctgatgtgccaacctattctgttgcttgaccaagttcttgtatcagacgttggagatagtactgaagtttccgttaagatgtttgatgcttatgtcgacaccttttcagcaacttttagtgttcctatggaaaaacttaaggcacttgttgctacagctcacagcgagttagcaaagggtgtagctttagatggtgtcctttctacattcgtgtcagctgcccgacaaggtgttgttgataccgatgttgacacaaaggatgttattgaatgtctcaaactttcacatcactctgacttagaagtgacaggtgacagttgtaacaatttcatgctcacctataataaggttgaaaacatgacgcccagagatcttggcgcatgtattgactgtaatgcaaggcatatcaatgcccaagtagcaaaaagtcacaatgtttcactcatctggaatgtaaaagactacatgtctttatctgaacagctgcgtaaacaaattcgtagtgctgccaagaagaacaacataccttttagactaacttgtgctacaactagacaggttgtcaatgtcataactactaaaatctcactcaagggtggtaagattgttagtacttgttttaaacttatgcttaaggccacattattgtgcgttcttgctgcattggtttgttatatcgttatgccagtacatacattgtcaatccatgatggttacacaaatgaaatcattggttacaaagccattcaggatggtgtcactcgtgacatcatttctactgatgattgttttgcaaataaacatgctggttttgacgcatggtttagccagcgtggtggttcatacaaaaatgacaaaagctgccctgtagtagctgctatcattacaagagagattggtttcatagtgcctggcttaccgggtactgtgctgagagcaatcaatggtgacttcttgcattttctacctcgtgtttttagtgctgttggcaacatttgctacacaccttccaaactcattgagtatagtgattttgctacctctgcttgcgttcttgctgctgagtgtacaatttttaaggatgctatgggcaaacctgtgccatattgttatgacactaatttgctagagggttctatttcttatagtgagcttcgtccagacactcgttatgtgcttatggatggttccatcatacagtttcctaacacttacctggagggttctgttagagtagtaacaacttttgatgctgagtactgtagacatggtacatgcgaaaggtcagaagtaggtatttgcctatctaccagtggtagatgggttcttaataatgagcattacagagctctatcaggagttttctgtggtgttgatgcgatgaatctcatagctaacatctttactcctcttgtgcaacctgtgggtgctttagatgtgtctgcttcagtagtggctggtggtattattgccatattggtgacttgtgctgcctactactttatgaaattcagacgtgtttttggtgagtacaaccatgttgttgctgctaatgcacttttgtttttgatgtctttcactatactctgtctggtaccagcttacagctttctgccgggagtctactcagtcttttacttgtacttgacattctatttcaccaatgatgtttcattcttggctcaccttcaatggtttgccatgttttctcctattgtgcctttttggataacagcaatctatgtattctgtatttctctgaagcactgccattggttctttaacaactatcttaggaaaagagtcatgtttaatggagttacatttagtaccttcgaggaggctgctttgtgtacctttttgctcaacaaggaaatgtacctaaaattgcgtagcgagacactgttgccacttacacagtataacaggtatcttgctctatataacaagtacaagtatttcagtggagccttagatactaccagctatcgtgaagcagcttgctgccacttagcaaaggctctaaatgactttagcaactcaggtgctgatgttctctaccaaccaccacagacatcaatcacttctgctgttctgcagagtggttttaggaaaatggcattcccgtcaggcaaagttgaagggtgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcagttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataacattaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaaggtaagttcaagaaaattgttaagggcactcatcattggatgcttttaactttcttgacatcactattgattcttgttcaaagtacacagtggtcactgtttttctttgtttacgagaatgctttcttgccatttactcttggtattatggcaattgctgcatgtgctatgctgcttgttaagcataagcacgcattcttgtgcttgtttctgttaccttctcttgcaacagttgcttactttaatatggtctacatgcctgctagctgggtgatgcgtatcatgacatggcttgaattggctgacactagcttgtctggttataggcttaaggattgtgttatgtatgcttcagctttagttttgcttattctcatgacagctcgcactgtttatgatgatgctgctagacgtgtttggacactgatgaatgtcattacacttgtttacaaagtctactatggtaatgctttagatcaagctatttccatgtgggccttagttatttctgtaacctctaactattctggtgtcgttacgactatcatgtttttagctagagctatagtgtttgtgtgtgttgagtattacccattgttatttattactggcaacaccttacagtgtatcatgcttgtttattgtttcttaggctattgttgctgctgctactttggccttttctgtttactcaaccgttacttcaggcttactcttggtgtttatgactacttggtctctacacaagaatttaggtatatgaactcccaggggcttttgcctcctaagagtagtattgatgctttcaagcttaacattaagttgttgggtattggaggtaaaccatgtatcaaggttgctactgtacagtctaaaatgtctgacgtaaagtgcacatctgtggtactgctctcggttcttcaacaacttagagtagagtcatcttctaaattgtgggcacaatgtgtacaactccacaatgatattcttcttgcaaaagacacaactgaagctttcgagaagatggtttctcttttgtctgttttgctatccatgcagggtgctgtagacattaataggttgtgcgaggaaatgctcgataaccgtgctactcttcaggctattgcttcagaatttagttctttaccatcatatgccgcttatgccactgcccaggaggcctatgagcaggctgtagctaatggtgattctgaagtcgttctcaaaaagttaaagaaatctttgaatgtggctaaatctgagtttgaccgtgatgctgccatgcaacgcaagttggaaaagatggcagatcaggctatgacccaaatgtacaaacaggcaagatctgaggacaagagggcaaaagtaactagtgctatgcaaacaatgctcttcactatgcttaggaagcttgataatgatgcacttaacaacattatcaacaatgcgcgtgatggttgtgttccactcaacatcataccattgactacagcagccaaactcatggttgttgtccctgattatggtacctacaagaacacttgtgatggtaacacctttacatatgcatctgcactctgggaaatccagcaagttgttgatgcggatagcaagattgttcaacttagtgaaattaacatggacaattcaccaaatttggcttggcctcttattgttacagctctaagagccaactcagctgttaaactacagaataatgaactgagtccagtagcactacgacagatgtcctgtgcggctggtaccacacaaacagcttgtactgatgacaatgcacttgcctactataacaattcgaagggaggtaggtttgtgctggcattactatcagaccaccaagatctcaaatgggctagattccctaagagtgatggtacaggtacaatttacacagaactggaaccaccttgtaggtttgttacagacacaccaaaagggcctaaagtgaaatacttgtacttcatcaaaggcttaaacaacctaaatagaggtatggtgctgggcagtttagctgctacagtacgtcttcaggctggaaatgctacagaagtacctgccaattcaactgtgctttccttctgtgcttttgcagtagaccctgctaaagcatataaggattacctagcaagtggaggacaaccaatcaccaactgtgtgaagatgttgtgtacacacactggtacaggacaggcaattactgtaacaccagaagctaacatggaccaagagtcctttggtggtgcttcatgttgtctgtattgtagatgccacattgaccatccaaatcctaaaggattctgtgacttgaaaggtaagtacgtccaaatacctaccacttgtgctaatgacccagtgggttttacacttagaaacacagtctgtaccgtctgcggaatgtggaaaggttatggctgtagttgtgaccaactccgcgaacccttgatgcagtctgcggatgcatcaacgtttttaaacgggtttgcggtgtaagtgcagcccgtcttacaccgtgcggcacaggcactagtactgatgtcgtctacagggcttttgatatttacaacgaaaaagttgctggttttgcaaagttcctaaaaactaattgctgtcgcttccaggagaaggatgaggaaggcaatttattagactcttactttgtagttaagaggcatactatgtctaactaccaacatgaagagactatttataacttggttaaagattgtccagcggttgctgtccatgactttttcaagtttagagtagatggtgacatggtaccacatatatcacgtcagcgtctaactaaatacacaatggctgatttagtctatgctctacgtcattttgatgagggtaattgtgatacattaaaagaaatactcgtcacatacaattgctgtgatgatgattatttcaataagaaggattggtatgacttcgtagagaatcctgacatcttacgcgtatatgctaacttaggtgagcgtgtacgccaatcattattaaagactgtacaattctgcgatgctatgcgtgatgcaggcattgtaggcgtactgacattagataatcaggatcttaatgggaactggtacgatttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatttgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacctgccttacccagatccatcaagaatattaggcgcaggctgttttgtcgatgatattgtcaaaacagatggtacacttatgattgaaaggttcgtgtcactggctattgatgcttacccacttacaaaacatcctaatcaggagtatgctgatgtctttcacttgtatttacaatacattagaaagttacatgatgagcttactggccacatgttggacatgtattccgtaatgctaactaatgataacacctcacggtactgggaacctgagttttatgaggctatgtacacaccacatacagtcttgcaggctgtaggtgcttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgctactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgccacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcattaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggataagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgcttcaatgtggctatcacaagggcaaaaattggcattttgtgcataatgtctgatagagatctttatgacaaactgcaatttacaagtctagaaataccacgtcgcaatgtggctacattacaagcagaaaatgtaactggactttttaaggactgtagtaagatcattactggtcttcatcctacacaggcacctacacacctcagcgttgatataaagttcaagactgaaggattatgtgttgacataccaggcataccaaaggacatgacctaccgtagactcatctctatgatgggtttcaaaatgaattaccaagtcaatggttaccctaatatgtttatcacccgcgaagaagctattcgtcacgttcgtgcgtggattggctttgatgtagagggctgtcatgcaactagagatgctgtgggtactaacctacctctccagctaggattttctacaggtgttaacttagtagctgtaccgactggttatgttgacactgaaaataacacagaattcaccagagttaatgcaaaacctccaccaggtgaccagtttaaacatcttataccactcatgtataaaggcttgccctggaatgtagtgcgtattaagatagtacaaatgctcagtgatacactgaaaggattgtcagacagagtcgtgttcgtcctttgggcgcatggctttgagcttacatcaatgaagtactttgtcaagattggacctgaaagaacgtgttgtctgtgtgacaaacgtgcaacttgcttttctacttcatcagatacttatgcctgctggaatcattctgtgggttttgactatgtctataacccatttatgattgatgttcagcagtggggctttacgggtaaccttcagagtaaccatgaccaacattgccaggtacatggaaatgcacatgtggctagttgtgatgctatcatgactagatgtttagcagtccatgagtgctttgttaagcgcgttgattggtctgttgaataccctattataggagatgaactgagggttaattctgcttgcagaaaagtacaacacatggttgtgaagtctgcattgcttgctgataagtttccagttcttcatgacattggaaatccaaaggctatcaagtgtgtgcctcaggctgaagtagaatggaagttctacgatgctcagccatgtagtgacaaagcttacaaaatagaggagctcttctattcttatgctacacatcacgataaattcactgatggtgtttgtttgttttggaattgtaacgttgatcgttacccagccaatgcaattgtgtgtaggtttgacacaagagtcttgtcaaacttgaacttaccaggctgtgatggtggtagtttgtatgtgaataagcatgcattccacactccagctttcgataaaagtgcatttactaatttaaagcaattgcctttcttttactattctgatagtccttgtgagtctcatggcaaacaagtagtgtcggatattgattatgttccactcaaatctgctacgtgtattacacgatgcaatttaggtggtgctgtttgcagacaccatgcaaatgagtaccgacagtacttggatgcatataatatgatgatttctgctggatttagcctatggatttacaaacaatttgatacttataacctgtggaatacatttaccaggttacagagtttagaaaatgtggcttataatgttgttaataaaggacactttgatggacacgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttcagtcatggacaacttggcggtcttcatttaatgataggcttagccaagcgctcacaagattcaccacttaaattagaggattttatccctatggacagcacagtgaaaaattacttcataacagatgcgcaaacaggttcatcaaaatgtgtgtgttctgtgattgatcttttacttgatgactttgtcgagataataaagtcacaagatttgtcagtgatttcaaaagtggtcaaggttacaattgactatgctgaaatttcattcatgctttggtgtaaggatggacatgttgaaaccttctacccaaaactacaagcaagtcaagcgtggcaaccaggtgttgcgatgcctaacttgtacaagatgcaaagaatgcttcttgaaaagtgtgaccttcagaattatggtgaaaatgctgttataccaaaaggaataatgatgaatgtcgcaaagtatactcaactgtgtcaatacttaaatacacttactttagctgtaccctacaacatgagagttattcactttggtgctggctctgataaaggagttgcaccaggtacagctgtgctcagacaatggttgccaactggcacactacttgtcgattcagatcttaatgacttcgtctccgacgcagattctactttaattggagactgtgcaacagtacatacggctaataaatgggaccttattattagcgatatgtatgaccctaggaccaaacatgtgacaaaagagaatgactctaaagaagggtttttcacttatctgtgtggatttataaagcaaaaactagccctgggtggttctatagctgtaaagataacagagcattcttggaatgctgacctttacaagcttatgggccatttctcatggtggacagcttttgttacaaatgtaaatgcatcatcatcggaagcatttttaattggggctaactatcttggcaagccgaaggaacaaattgatggctataccatgcatgctaactacattttctggaggaacacaaatcctatccagttgtcttcctattcactctttgacatgagcaaatttcctcttaaattaagaggaactgctgtaatgtctcttaaggagaatcaaatcaatgatatgatttattctcttctggaaaaaggtaggcttatcattagagaaaacaacagagttgtggtttcaagtgatattcttgttaacaactaaacgaacatgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagctcgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataaacgaacttatggatttgtttatgagattttttactcttggatcaattactgcacagccagtaaaaattgacaatgcttctcctgcaagtactgttcatgctacagcaacgataccgctacaagcctcactccctttcggatggcttgttattggcgttgcatttcttgctgtttttcagagcgctaccaaaataattgcgctcaataaaagatggcagctagccctttataagggcttccagttcatttgcaatttactgctgctatttgttaccatctattcacatcttttgcttgtcgctgcaggtatggaggcgcaatttttgtacctctatgccttgatatattttctacaatgcatcaacgcatgtagaattattatgagatgttggctttgttggaagtgcaaatccaagaacccattactttatgatgccaactactttgtttgctggcacacacataactatgactactgtataccatataacagtgtcacagatacaattgtcgttactgaaggtgacggcatttcaacaccaaaactcaaagaagactaccaaattggtggttattctgaggataggcactcaggtgttaaagactatgtcgttgtacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgctaatccagcaatggatccaatttatgatgagccgacgacgactactagcgtgcctttgtaagcacaagaaagtgagtacgaacttatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacaccccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaagtgacaacagatgtttcatcttgttgacttccaggttacaatagcagagatattgattatcattatgaggactttcaggattgctatttggaatcttgacgttataataagttcaatagtgagacaattatttaagcctctaactaagaagaattattcggagttagatgatgaagaacctatggagttagattatccataaaacgaacatgaaaattattctcttcctgacattgattgtatttacatcttgcgagctatatcactatcaggagtgtgttagaggtacgactgtactactaaaagaaccttgcccatcaggaacatacgagggcaattcaccatttcaccctcttgctgacaataaatttgcactaacttgcactagcacacactttgcttttgcttgtgctgacggtactcgacatacctatcagctgcgtgcaagatcagtttcaccaaaacttttcatcagacaagaggaggttcaacaagagctctactcgccactttttctcattgttgctgctctagtatttttaatactttgcttcaccattaagagaaagacagaatgaatgagctcactttaattgacttctatttgtgctttttagcctttctgctattccttgttttaataatgcttattatattttggttttcactcgaaatccaggatctagaagaaccttgtaccaaagtctaaacgaacatgaaacttctcattgttttgacttgtatttctctatgcagttgcatatgcactgtagtacagcgctgtgcatctaataaacctcatgtgcttgaagatccttgtaaggtacaacactaggggtaatacttatagcactgcttggctttgtgctctaggaaaggttttaccttttcatagatggcacactatggttcaaacatgcacacctaatgttactatcaactgtcaagatccagctggtggtgcgcttatagctaggtgttggtaccttcatgaaggtcaccaaactgctgcatttagagacgtacttgttgttttaaataaacgaacaaattaaaatgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataaacactcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgac

SEQ ID NO: 16 - DNA, 29742 bp, SARS Coronavirus
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgtgagctcaatggaggtgcagtcactcgctatgtcgacaacaatttctgtggcccagatgggtaccctcttgattgcatcaaagattttctcgcacgcgcgggcaagtcaatgtgcactctttccgaacaacttgattacatcgagtcgaagagaggtgtctactgctgccgtgaccatgagcatgaaattgcctggttcactgagcgctctgataagagctacgagcaccagacacccttcgaaattaagagtgccaagaaatttgacactttcaaaggggaatgcccaaagtttgtgtttcctcttaactcaaaagtcaaagtcattcaaccacgtgttgaaaagaaaaagactgagggtttcatggggcgtatacgctctgtgtaccctgttgcatctccacaggagtgtaacaatatgcacttgtctaccttgatgaaatgtaatcattgcgatgaagtttcatggcagacgtgcgactttctgaaagccacttgtgaacattgtggcactgaaaatttagttattgaaggacctactacatgtgggtacctacctactaatgctgtagtgaaaatgccatgtcctgcctgtcaagacccagagattggacctgagcatagtgttgcagattatcacaaccactcaaacattgaaactcgactccgcaagggaggtaggactagatgttttggaggctgtgtgtttgcctatgttggctgctataataagcgtgcctactgggttcctcgtgctagtgctgatattggctcaggccatactggcattactggtgacaatgtggagaccttgaatgaggatctccttgagatactgagtcgtgaacgtgttaacattaacattgttggcgattttcatttgaatgaagaggttgccatcattttggcatctttctctgcttctacaagtgcctttattgacactataaagagtcttgattacaagtctttcaaaaccattgttgagtcctgcggtaactataaagttaccaagggaaagcccgtaaaaggtgcttggaacattggacaacagagatcagttttaacaccactgtgtggttttccctcacaggctgctggtgttatcagatcaatttttgcgcgcacacttgatgcagcaaaccactcaattcctgatttgcaaagagcagctgtcaccatacttgatggtatttctgaacagtcattacgtcttgtcgacgccatggtttatacttcagacctgctcaccaacagtgtcattattatggcatatgtaactggtggtcttgtacaacagacttctcagtggttgtctaatcttttgggcactactgttgaaaaactcaggcctatctttgaatggattgaggcgaaacttagtgcaggagttgaatttctcaaggatgcttgggagattctcaaatttctcattacaggtgtttttgacatcgtcaagggtcaaatacaggttgcttcagataacatcaaggattgtgtaaaatgcttcattgatgttgttaacaaggcactcgaaatgtgcattgatcaagtcactatcgctggcgcaaagttgcgatcactcaacttaggtgaagtcttcatcgctcaaagcaagggactttaccgtcagtgtatacgtggcaaggagcagctgcaactactcatgcctcttaaggcaccaaaagaagtaacctttcttgaaggtgattcacatgacacagtacttacctctgaggaggttgttctcaagaacggtgaactcgaagcactcgagacgcccgttgatagcttcacaaatggagctatcgtcggcacaccagtctgtgtaaatggcctcatgctcttagagattaaggacaaagaacaatactgcgcattgtctcctggtttactggctacaaacaatgtctttcgcttaaaagggggtgcaccaattaaaggtgtaacctttggagaagatactgtttgggaagttcaaggttacaagaatgtgagaatcacatttgagcttgatgaacgtgttgacaaagtgcttaatgaaaagtgctctgtctacactgttgaatccggtaccgaagttactgagtttgcatgtgttgtagcagaggctgttgtgaagactttacaaccagtttctgatctccttaccaacatgggtattgatcttgatgagtggagtgtagctacattctacttatttgatgatgctggtgaagaaaacttttcatcacgtatgtattgttccttttaccctccagatgaggaagaagaggacgatgcagagtgtgaggaagaagaaattgatgaaacctgtgaacatgagtacggtacagaggatgattatcaaggtctccctctggaatttggtgcctcagctgaaacagttcgagttgaggaagaagaagaggaagactggctggatgatactactgagcaatcagagattgagccagaaccagaacctacacctgaagaaccagttaatcagtttactggttatttaaaacttactgacaatgttgccattaaatgtgttgacatcgttaaggaggcacaaagtgctaatcctatggtgattgtaaatgctgctaacatacacctgaaacatggtggtggtgtagcaggtgcactcaacaaggcaaccaatggtgccatgcaaaaggagagtgatgattacattaagctaaatggccctcttacagtaggagggtcttgtttgctttctggacataatcttgctaagaagtgtctgcatgttgttggacctaacctaaatgcaggtgaggacatccagcttcttaaggcagcatatgaaaatttcaattcacaggacatcttacttgcaccattgttgtcagcaggcatatttggtgctaaaccacttcagtctttacaagtgtgcgtgcagacggttcgtacacaggtttatattgcagtcaatgacaaagctctttatgagcaggttgtcatggattatcttgataacctgaagcctagagtggaagcacctaaacaagaggagccaccaaacacagaagattccaaaactgaggagaaatctgtcgtacagaagcctgtcgatgtgaagccaaaaattaaggcctgcattgatgaggttaccacaacactggaagaaactaagtttcttaccaataagttactcttgtttgctgatatcaatggtaagctttaccatgattctcagaacatgcttagaggtgaagatatgtctttccttgagaaggatgcaccttacatggtaggtgatgttatcactagtggtgatatcacttgtgttgtaataccctccaaaaaggctggtggcactactgagatgctctcaagagctttgaagaaagtgccagttgatgagtatataaccacgtaccctggacaaggatgtgctggttatacacttgaggaagctaagactgctcttaagaaatgcaaatctgcattttatgtactaccttcagaagcacctaatgctaaggaagagattctaggaactgtatcctggaatttgagagaaatgcttgctcatgctgaagagacaagaaaattaatgcctatatgcatggatgttagagccataatggcaaccatccaacgtaagtataaaggaattaaaattcaagagggcatcgttgactatggtgtccgattcttcttttatactagtaaagagcctgtagcttctattattacgaagctgaactctctaaatgagccgcttgtcacaatgccaattggttatgtgacacatggttttaatcttgaagaggctgcgcgctgtatgcgttctcttaaagctcctgccgtagtgtcagtatcatcaccagatgctgttactacatataatggatacctcacttcgtcatcaaagacatctgaggagcactttgtagaaacagtttctttggctggctcttacagagattggtcctattcaggacagcgtacagagttaggtgttgaatttcttaagcgtggtgacaaaattgtgtaccacactctggagagccccgtcgagtttcatcttgacggtgaggttctttcacttgacaaactaaagagtctcttatccctgcgggaggttaagactataaaagtgttcacaactgtggacaacactaatctccacacacagcttgtggatatgtctatgacatatggacagcagtttggtccaacatacttggatggtgctgatgttacaaaaattaaacctcatgtaaatcatgagggtaagactttctttgtactacctagtgatgacacactacgtagtgaagctttcgagtactaccatactcttgatgagagttttcttggtaggtacatgtctgctttaaaccacacaaagaaatggaaatttcctcaagttggtggtttaacttcaattaaatgggctgataacaattgttatttgtctagtgttttattagcacttcaacagcttgaagtcaaattcaatgcaccagcacttcaagaggcttattatagagcccgtgctggtgatgctgctaacttttgtgcactcatactcgcttacagtaataaaactgttggcgagcttggtgatgtcagagaaactatgacccatcttctacagcatgctaatttggaatctgcaaagcgagttcttaatgtggtgtgtaaacattgtggtcagaaaactactaccttaacgggtgtagaagctgtgatgtatatgggtactctatcttatgataatcttaagacaggtgtttccattccatgtgtgtgtggtcgtgatgctacacaatatctagtacaacaagagtcttcttttgttatgatgtctgcaccacctgctgagtataaattacagcaaggtacattcttatgtgcgaatgagtacactggtaactatcagtgtggtcattacactcatataactgctaaggagaccctctatcgtattgacggagctcaccttacaaagatgtcagagtacaaaggaccagtgactgatgttttctacaaggaaacatcttacactacaaccatcaagcctgtgtcgtataaactcgatggagttacttacacagagattgaaccaaaattggatgggtattataaaaaggataatgcttactatacagagcagcctatagaccttgtaccaactcaaccattaccaaatgcgagttttgataatttcaaactcacatgttctaacacaaaatttgctgatgatttaaatcaaatgacaggcttcacaaagccagcttcacgagagctatctgtcacattcttcccagacttgaatggcgatgtagtggctattgactatagacactattcagcgagtttcaagaaaggtgctaaattactgcataagccaattgtttggcacattaaccaggctacaaccaagacaacgttcaaaccaaacacttggtgtttacgttgtctttggagtacaaagccagtagatacttcaaattcatttgaagttctggcagtagaagacacacaaggaatggacaatcttgcttgtgaaagtcaacaacccacctctgaagaagtagtggaaaatcctaccatacagaaggaagtcatagagtgtgacgtgaaaactaccgaagttgtaggcaatgtcatacttaaaccatcagatgaaggtgttaaagtaacacaagagttaggtcatgaggatcttatggctgcttatgtggaaaacacaagcattaccattaagaaacctaatgagctttcactagccttaggtttaaaaacaattgccactcatggtattgctgcaattaatagtgttccttggagtaaaattttggcttatgtcaaaccattcttaggacaagcagcaattacaacatcaaattgcgctaagagattagcacaacgtgtgtttaacaattatatgccttatgtgtttacattattgttccaattgtgtacttttactaaaagtaccaattctagaattagagcttcactacctacaactattgctaaaaatagtgttaagagtgttgctaaattatgtttggatgccggcattaattatgtgaagtcacccaaattttctaaattgttcacaatcgctatgtggctattgttgttaagtatttgcttaggttctctaatctgtgtaactgctgcttttggtgtactcttatctaattttggtgctccttcttattgtaatggcgttagagaattgtatcttaattcgtctaacgttactactatggatttctgtgaaggttcttttccttgcagcatttgtttaagtggattagactcccttgattcttatccagctcttgaaaccattcaggtgacgatttcatcgtacaagctagacttgacaattttaggtctggccgctgagtgggttttggcatatatgttgttcacaaaattcttttatttattaggtctttcagctataatgcaggtgttctttggctattttgctagtcatttcatcagcaattcttggctcatgtggtttatcattagtattgtacaaatggcacccgtttctgcaatggttaggatgtacatcttctttgcttctttctactacatatggaagagctatgttcatatcatggatggttgcacctcttcgacttgcatgatgtgctataagcgcaatcgtgccacacgcgttgagtgtacaactattgttaatggcatgaagagatctttctatgtctatgcaaatggaggccgtggcttctgcaagactcacaattggaattgtctcaattgtgacacattttgcactggtagtacattcattagtgatgaagttgctcgtgatttgtcactccagtttaaaagaccaatcaaccctactgaccagtcatcgtatattgttgatagtgttgctgtgaaaaatggcgcgcttcacctctactttgacaaggctggtcaaaagacctatgagagacatccgctctcccattttgtcaatttagacaatttgagagctaacaacactaaaggttcactgcctattaatgtcatagtttttgatggcaagtccaaatgcgacgagtctgcttctaagtctgcttctgtgtactacagtcagctgatgtgccaacctattctgttgcttgaccaagctcttgtatcaaacgttggagatagtactgaagtttccgttaagatgtttgatgcttatgtcgacaccttttcagcaacttttagtgttcctatggaaaaacttaaggcacttgttgctacagctcacagcgagttagcaaagggtgtagctttagatggtgtcctttctacattcgtgtcagctgcccgacaaggtgttgttgataccgatgttgacacaaaggatgttattgaatgtctcaaactttcacatcactctgacttagaagtgacaggtgacagttgtaacaatttcatgctcacctataataaggttgaaaacatgacgcccagagatcttggcgcatgtattgactgtaatgcaaggcatatcaatgcccaagtagcaaaaagtcacaatgtttcactcatctggaatgtaaaagactacatgtctttatctgaacagctgcgtaaacaaattcgtactgctgccaagaagaacaacataccttttacactaacttgtgctacaactagacaggttgtcaatgtcataactactaaaatctcactcaagggtggtaagattgttagtacttgttttaaacttatgcttaaggccacattattgtgcgttcttgctgcattggtttgttatatcgttatgccagtacatacattgtcaatccatgatggttacacaaatgaaatcattggttacaaagccattcaggatggtgtcactcgtgacatcatttctactgatgattgttttgcaaataaacatgctggttttgacgcatggtttagccagcgtggtggttcatacaaaaatgacaaaagctgccctgtagtagctgctatcattacaagagagattggtttcatagtgcctggcttaccgggtactgtgctgagagcaatcaatggtgacttcttgcattttctacctcgtgtttttagtgctgttggcaacatttgctacacaccttccaaactcattgagtatagtgattttgctacctctgcttgcgttcttgctgctgagtgtacaatttttaaggatgctatgggcaaacctgtgccatattgttatgacactaatttgctagagggttctatttcttatagtgagcttcgtccagacactcgttatgtgcttatggatggttccatcatacagtttcctaacacttacctggagggttctgttagagtagtaacaacttttgatgctgagtactgtagacatggtacatgcgaaaggtcagaagtaggtatttgcctatctaccagtggtagatgggttcttaataatgagcattacagagctctatcaggagttttctgtggtgttgatgcgatgaatctcatagctaacatctttactcctcttgtgcaacctgtgggtgctttagatgtgtctgcttcagtagtggctggtggtattattgccatattggtgacttgtgctgcctactactttatgaaattcagacgtgtttttggtgagtacaaccatgttgttgctgctaatgcacttttgtttttgatgtctttcactatactctgtctggtaccagcttacagctttctgccgggagtctactcagtcttttacttgtacttgacattctatttcaccaatgatgtttcattcttggctcaccttcaatggtttgccatgttttctcctattgtgcctttttggataacagcaatctatgtattctgtatttctctgaagcactgccattggttctttaacaactatcttaggaaaagagtcatgtttaatggagttacatttagtaccttcgaggaggctgctttgtgtacctttttgctcaacaaggaaatgtacctaaaattgcgtagcgagacactgttgccacttacacagtataacaggtatcttgctctatataacaagtacaagtatttcagtggagccttagatactaccagctatcgtgaagcagcttgctgccacttagcaaaggctctaaatgactttagcaactcaggtgctgatgttctctaccaaccaccacagacatcaatcacttctgctgttctgcagagtggttttaggaaaatggcattcccgtcaggcaaagttgaagggtgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcagttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataacattaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaaggtaagttcaagaaaattgttaagggcactcatcattggatgcttttaactttcttgacatcactattgattcttgttcaaagtacacagtggtcactgtttttctttgtttacgagaatgctttcttgccatttactcttggtattatggcaattgctgcatgtgctatgctgcttgttaagcataagcacgcattcttgtgcttgtttctgttaccttctcttgcaacagttgcttactttaatatggtctacatgcctgctagctgggtgatgcgtatcatgacatggcttgaattggctgacactagcttgtctggttataggcttaaggattgtgttatgtatgcttcagctttagttttgcttattctcatgacagctcgcactgtttatgatgatgctgctagacgtgtttggacactgatgaatgtcattacacttgtttacaaagtctactatggtaatgctttagatcaagctatttccatgtgggccttagttatttctgtaacctctaactattctggtgtcgttacgactatcatgtttttagctagagctatagtgtttgtgtgtgttgagtattacccattgttatttattactggcaacaccttacagtgtatcatgcttgtttattgtttcttaggctattgttgctgctgctactttggccttttctgtttactcaaccgttacttcaggcttactcttggtgtttatgactacttggtctctacacaagaatttaggtatatgaactcccaggggcttttgcctcctaagagtagtattgatgctttcaagcttaacattaagttgttgggtattggaggtaaaccatgtatcaaggttgctactgtacagtctaaaatgtctgacgtaaagtgcacatctgtggtactgctctcggttcttcaacaacttagagtagagtcatcttctaaattgtgggcacaatgtgtacaactccacaatgatattcttcttgcaaaagacacaactgaagctttcgagaagatggtttctcttttgtctgttttgctatccatgcagggtgctgtagacattaataggttgtgcgaggaaatgctcgataaccgtgctactcttcaggctattgcttcagaatttagttctttaccatcatatgccgcttatgccactgcccaggaggcctatgagcaggctgtagctaatggtgattctgaagtcgttctcaaaaagttaaagaaatctttgaatgtggctaaatctgagtttgaccgtgatgctgccatgcaacgcaagttggaaaagatggcagatcaggctatgacccaaatgtacaaacaggcaagatctgaggacaagagggcaaaagtaactagtgctatgcaaacaatgctcttcactatgcttaggaagcttgataatgatgcacttaacaacattatcaacaatgcgcgtgatggttgtgttccactcaacatcataccattgactacagcagccaaactcatggttgttgtccctgattatggtacctacaagaacacttgtgatggtaacacctttacatatgcatctgcactctgggaaatccagcaagttgttgatgcggatagcaagattgttcaacttagtgaaattaacatggacaattcaccaaatttggcttggcctcttattgttacagctctaagagccaactcagctgttaaactacagaataatgaactgagtccagtagcactacgacagatgtcctgtgcggctggtaccacacaaacagcttgtactgatgacaatgcacttgcctactataacaattcgaagggaggtaggtttgtgctggcattactatcagaccaccaagatctcaaatgggctagattccctaagagtgatggtacaggtacaatttacacagaactggaaccaccttgtaggtttgttacagacacaccaaaagggcctaaagtgaaatacttgtacttcatcaaaggcttaaacaacctaaatagaggtatggtgctgggcagtttagctgctacagtacgtcttcaggctggaaatgctacagaagtacctgccaattcaactgtgctttccttctgtgcttttgcagtagaccctgctaaagcatataaggattacctagcaagtggaggacaaccaatcaccaactgtgtgaagatgttgtgtacacacactggtacaggacaggcaattactgtaacaccagaagctaacatggaccaagagtcctttggtggtgcttcatgttgtctgtattgtagatgccacattgaccatccaaatcctaaaggattctgtgacttgaaaggtaagtacgtccaaatacctaccacttgtgctaatgacccagtgggttttacacttagaaacacagtctgtaccgtctgcggaatgtggaaaggttatggctgtagttgtgaccaactccgcgaacccttgatgcagtctgcggatgcatcaacgtttttaaacgggtttgcggtgtaagtgcagcccgtcttacaccgtgcggcacaggcactagtactgatgtcgtctacagggcttttgatatttacaacgaaaaaagtgctggttttgcaaagttcctaaaaactaattgctgtcgcttccaggagaaggatgaggaaggcaatttattagactcttactttgtagttaagaggcatactatgtctaactaccaacatgaagagactatttataacttggttaaagattgtccagcggttgctgtccatgactttttcaagtttagagtagatggtgacatggtaccacatatatcacgtcagcgtctaactaaatacacaatggctgatttagtctatgctctacgtcattttgatgagggtaattgtgatacattaaaagaaatactcgtcacatacaattgctgtgatgatgattatttcaataagaaggattggtatgacttcgtagagaatcctgacatcttacgcgtatatgctaacttaggtgagcgtgtacgccaatcattattaaagactgtacaattctgcgatgctatgcgtgatgcaggcattgtaggcgtactgacattagataatcaggatcttaatgggaactggtacgatttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatttgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacctgccttacccagatccatcaagaatattaggcgcaggctgttttgtcgatgatattgtcaaaacagatggtacacttatgattgaaaggttcgtgtcactggctattgatgcttacccacttacaaaacatcctaatcaggagtatgctgatgtctttcacttgtatttacaatacattagaaagttacatgatgagcttactggccacatgttggacatgtattccgtaatgctaactaatgataacacctcacggtactgggaacctgagttttatgaggctatgtacacaccacatacagtcttgcaggctgtaggtgcttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgccactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgccacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcattaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggataagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgcttcaatgtggctatcacaagggcaaaaattggcattttgtgcataatgtctgatagagatctttatgacaaactgcaatttacaagtctagaaataccacgtcgcaatgtggctacattacaagcagaaaatgtaactggactttttaaggactgtagtaagatcattactggtcttcatcctacacaggcacctacacacctcagcgttgatataaaattcaagactgaaggattatgtgttgacataccaggcataccaaaggacatgacctaccgtagactcatctctatgatgggtttcaaaatgaattaccaagtcaatggttaccctaatatgtttatcacccgcgaagaagctattcgtcacgttcgtgcgtggattggctttgatgtagagggctgtcatgcaactagagatgctgtgggtactaacctacctctccagctaggattttctacaggtgttaacttagtagctgtaccgactggttatgttgacactgaaaataacacagaattcaccagagttaatgcaaaacctccaccaggtgaccagtttaaacatcttataccactcatgtataaaggcttgccctggaatgtagtgcgtattaagatagtacaaatgctcagtgatacactgaaaggattgtcagacagagtcgtgttcgtcctttgggcgcatggctttgagcttacatcaatgaagtactttgtcaagattggacctgaaagaacgtgttgtctgtgtgacaaacgtgcaacttgcttttctacttcatcagatacttatgcctgctggaatcattctgtgggttttgactatgtctataacccatttatgattgatgttcagcagtggggctttacgggtaaccttcagagtaaccatgaccaacattgccaggtacatggaaatgcacatgtggctagttgtgatgctatcatgactagatgtttagcagtccatgagtgctttgttaagcgcgttgattggtctgttgaataccctattataggagatgaactgagggttaattctgcttgcagaaaagtacaacacatggttgtgaagtctgcattgcttgctgataagtttccagttcttcatgacattggaaatccaaaggctatcaagtgtgtgcctcaggctgaagtagaatggaagttctacgatgctcagccatgtagtgacaaagcttacaaaatagaggaactcttctattcttatgctacacatcacgataaattcactgatggtgtttgtttgttttggaattgtaacgttgatcgttacccagccaatgcaattgtgtgtaggtttgacacaagagtcttgtcaaacttgaacttaccaggctgtgatggtggtagtttgtatgtgaataagcatgcattccacactccagctttcgataaaagtgcatttactaatttaaagcaattgcctttcttttactattctgatagtccttgtgagtctcatggcaaacaagtagtgtcggatattgattatgttccactcaaatctgctacgtgtattacacgatgcaatttaggtggtgctgtttgcagacaccatgcaaatgagtaccgacagtacttggatgcatataatatgatgatttctgctggatttagcctatggatttacaaacaatttgatacttataacctgtggaatacatttaccaggttacagagtttagaaaatgtggcttataatgttgttaataaaggacactttgatggacacgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttcagtcatggacaacttggcggtcttcatttaatgataggcttagccaagcgctcacaagattcaccacttaaattagaggattttatccctatggacagcacagtgaaaaattacttcataacagatgcgcaaacaggttcatcaaaatgtgtgtgttctgtgattgatcttttacttgatgactttgtcgagataataaagtcacaagatttgtcagtgatttcaaaagtggtcaaggttacaattgactatgctgaaatttcattcatgctttggtgtaaggatggacatgttgaaaccttctacccaaaactacaagcaagtcaagcgtggcaaccaggtgttgcgatgcctaacttgtacaagatgcaaagaatgcttcttgaaaagtgtgaccttcagaattatggtgaaaatgctgttataccaaaaggaataatgatgaatgtcgcaaagtatactcaactgtgtcaatacttaaatacacttactttagctgtaccctacaacatgagagttattcactttggtgctggctctgataaaggagttgcaccaggtacagctgtgctcagacaatggttgccaactggcacactacttgtcgattcagatcttaatgacttcgtctccgacgcagattctactttaattggagactgtgcaacagtacatacggctaataaatgggaccttattattagcgatatgtatgaccctaggaccaaacatgtgacaaaagagaatgactctaaagaagggtttttcacttatctgtgtggatttataaagcaaaaactagccctgggtggttctatagctgtaaagataacagagcattcttggaatgctgacctttacaagcttatgggccatttctcatggtggacagcttttgttacaaatgtaaatgcatcatcatcggaagcatttttaattggggctaactatcttggcaagccgaaggaacaaattgatggctataccatgcatgctaactacattttctggaggaacacaaatcctatccagttgtcttcctattcactctttgacatgagcaaatttcctcttaaattaagaggaactgctgtaatgtctcttaaggagaatcaaatcaatgatatgatttattctcttctggaaaaaggtaggcttatcattagagaaaacaacagagttgtggtttcaagtgatattcttgttaacaactaaacgaacatgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataaacgaacttatggatttgtttatgagattttttactcttggatcaattactgcacagccagtaaaaattgacaatgcttctcctgcaagtactgttcatgctacagcaacgataccgctacaagcctcactccctttcggatggcttgttattggcgttgcatttcttgctgtttttcagagcgctaccaaaataattgcgctcaataaaagatggcagctagccctttataagggcttccagttcatttgcaatttactgctgctatttgttaccatctattcacatcttttgcttgtcgctgcaggtaaggaggcgcaatttttgtacctctatgccttgatatattttctacaatgcatcaacgcatgtagaattattatgagatgttggctttgttggaagtgcaaatccaagaacccattactttatgatgccaactactttgtttgctggcacacacataactatgactactgtataccatataacagtgtcacagatacaattgtcgttactgaaggtgacggcatttcaacaccaaaactcaaagaagactaccaaattggtggttattctgaggataggcactcaggtgttaaagactatgtcgttgtacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgctaatccagcaatggatccaatttatgatgagccgacgacgactactagcgtgcctttgtaagcacaagaaagtgagtacgaacttatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgttgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacactccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaagtgacaacagatgtttcatcttgttgacttccaggttacaatagcagagatattgattatcattatgaggactttcaggattgctatttggaatcttgacgttataataagttcaatagtgagacaattatttaagcctctaactaagaagaattattcggagttagatgatgaagaacctatggagttagattatccataaaacgaacatgaaaattattctcttcctgacattgattgtatttacatcttgcgagctatatcactatcaggagtgtgttagaggtacgactgtactactaaaagaaccttgcccatcaggaacatacgagggcaattcaccatttcaccctcttgctgacaataaatttgcactaacttgcactagcacacactttgcttttgcttgtgctgacggtactcgacatacctatcagctgcgtgcaagatcagtttcaccaaaacttttcatcagacaagaggaggttcaacaagagctctactcgccactttttctcattgttgctgctctagtatttttaatactttgcttcaccattaagagaaagacagaatgaatgagctcactttaattgacttctatttgtgctttttagcctttctgctattccttgttttaataatgcttattatattttggttttcactcgaaatccaggatctagaagaaccttgtaccaaagtctaaacgaacatgaaacttctcattgttttgacttgtatttctctatgcagttgcatatgcactgtagtacagcgctgtgcatctaataaacctcatgtgcttgaagatccttgtaaggtacaacactaggggtaatacttatagcactgcttggctttgtgctctaggaaaggttttaccttttcatagatggcacactatggttcaaacatgcacacctaatgttactatcaactgtcaagatccagctggtggtgcgcttatagctaggtgttggtaccttcatgaaggtcaccaaactgctgcatttagagacgtacttgttgttttaaataaacgaacaaattaaaatgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataaacactcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgacaaaaaaaaaaaaaaa

SEQ ID NO: 17 - DNA, 29736 bp, SARS Coronavirus
ctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgtgagctcaatggaggtgcagtcactcgctatgtcgacaacaatttctgtggcccagatgggtaccctcttgattgcatcaaagattttctcgcacgcgcgggcaagtcaatgtgcactctttccgaacaacttgattacatcgagtcgaagagaggtgtctactgctgccgtgaccatgagcatgaaattgcctggttcactgagcgctctgataagagctacgagcaccagacacccttcgaaattaagagtgccaagaaatttgacactttcaaaggggaatgcccaaagtttgtgtttcctcttaactcaaaagtcaaagtcattcaaccacgtgttgaaaagaaaaagactgagggtttcatggggcgtatacgctctgtgtaccctgttgcatctccacaggagtgtaacaatatgcacttgtctaccttgatgaaatgtaatcattgcgatgaagtttcatggcagacgtgcgactttctgaaagccacttgtgaacattgtggcactgaaaatttagttattgaaggacctactacatgtgggtacctacctactaatgctgtagtgaaaatgccatgtcctgcctgtcaagacccagagattggacctgagcatagtgttgcagattatcacaaccactcaaacattgaaactcgactccgcaagggaggtaggactagatgttttggaggctgtgtgtttgcctatgttggctgctataataagcgtgcctactgggttcctcgtgctagtgctgatattggctcaggccatactggcattactggtgacaatgtggagaccttgaatgaggatctccttgagatactgagtcgtgaacgtgttaacattaacattgttggcgattttcatttgaatgaagaggttgccatcattttggcatctttctctgcttctacaagtgcctttattgacactataaagagtcttgattacaagtctttcaaaaccattgttgagtcctgcggtaactataaagttaccaagggaaagcccgtaaaaggtgcttggaacattggacaacagagatcagttttaacaccactgtgtggttttccctcacaggctgctggtgttatcagatcaatttttgcgcgcacacttgatgcagcaaaccactcaattcctgatttgcaaagagcagctgtcaccatacttgatggtatttctgaacagtcattacgtcttgtcgacgccatggtttatacttcagacctgctcaccaacagtgtcattattatggcatatgtaactggtggtcttgtacaacagacttctcagtggttgtctaatcttttgggcactactgttgaaaaactcaggcctatctttgaatggattgaggcgaaacttagtgcaggagttgaatttctcaaggatgcttgggagattctcaaatttctcattacaggtgtttttgacatcgtcaagggtcaaatacaggttgcttcagataacatcaaggattgtgtaaaatgcttcattgatgttgttaacaaggcactcgaaatgtgcattgatcaagtcactatcgctggcgcaaagttgcgatcactcaacttaggtgaagtcttcatcgctcaaagcaagggactttaccgtcagtgtatacgtggcaaggagcagctgcaactactcatgcctcttaaggcaccaaaagaagtaacctttcttgaaggtgattcacatgacacagtacttacctctgaggaggttgttctcaagaacggtgaactcgaagcactcgagacgcccgttgatagcttcacaaatggagctatcgttggcacaccagtctgtgtaaatggcctcatgctcttagagattaaggacaaagaacaatactgcgcattgtctcctggtttactggctacaaacaatgtctttcgcttaaaagggggtgcaccaattaaaggtgtaacctttggagaagatactgtttgggaagttcaaggttacaagaatgtgagaatcacatttgagcttgatgaacgtgttgacaaagtgcttaatgaaaagtgctctgtctacactgttgaatccggtaccgaagttactgagtttgcatgtgttgtagcagaggctgttgtgaagactttacaaccagtttctgatctccttaccaacatgggtattgatcttgatgagtggagtgtagctacattctacttatttgatgatgctggtgaagaaaacttttcatcacgtatgtattgttccttttaccctccagatgaggaagaagaggacgatgcagagtgtgaggaagaagaaattgatgaaacctgtgaacatgagtacggtacagaggatgattatcaaggtctccctctggaatttggtgcctcagctgaaacagttcgagttgaggaagaagaagaggaagactggctggatgatactactgagcaatcagagattgagccagaaccagaacctacacctgaagaaccagttaatcagtttactggttatttaaaacttactgacaatgttgccattaaatgtgttgacatcgttaaggaggcacaaagtgctaatcctatggtgattgtaaatgctgctaacatacacctgaaacatggtggtggtgtagcaggtgcactcaacaaggcaaccaatggtgccatgcaaaaggagagtgatgattacattaagctaaatggccctcttacagtaggagggtcttgtttgctttctggacataatcttgctaagaagtgtctgcatgttgttggacctaacctaaatgcaggtgaggacatccagcttcttaaggcagcatatgaaaatttcaattcacaggacatcttacttgcaccattgttgtcagcaggcatatttggtgctaaaccacttcagtctttacaagtgtgcgtgcagacggttcgtacacaggtttatattgcagtcaatgacaaagctctttatgagcaggttgtcatggattatcttgataacctgaagcctagagtggaagcacctaaacaagaggagccaccaaacacagaagattccaaaactgaggagaaatctgtcgtacagaagcctgtcgatgtgaagccaaaaattaaggcctgcattgatgaggttaccacaacactggaagaaactaagtttcttaccaataagttactcttgtttgctgatatcaatggtaagctttaccatgattctcagaacatgcttagaggtgaagatatgtctttccttgagaaggatgcaccttacatggtaggtgatgttatcactagtggtgatatcacttgtgttgtaataccctccaaaaaggctggtggcactactgagatgctctcaagagctttgaagaaagtgccagttgatgagtatataaccacgtaccctggacaaggatgtgctggttatacacttgaggaagctaagactgctcttaagaaatgcaaatctgcattttatgtactaccttcagaagcacctaatgctaaggaagagattctaggaactgtatcctggaatttgagagaaatgcttgctcatgctgaagagacaagaaaattaatgcctatatgcatggatgttagagccataatggcaaccatccaacgtaagtataaaggaattaaaattcaagagggcatcgttgactatggtgtccgattcttcttttatactagtaaagagcctgtagcttctattattacgaagctgaactctctaaatgagccgcttgtcacaatgccaattggttatgtgacacatggttttaatcttgaagaggctgcgcgctgtatgcgttctcttaaagctcctgccgtagtgtcagtatcatcaccagatgctgttactacatataatggatacctcacttcgtcatcaaagacatctgaggagcactttgtagaaacagtttctttggctggctcttacagagattggtcctattcaggacagcgtacagagttaggtgttgaatttcttaagcgtggtgacaaaattgtgtaccacactctggagagccccgtcgagtttcatcttgacggtgaggttctttcacttgacaaactaaagagtctcttatccctgcgggaggttaagactataaaagtgttcacaactgtggacaacactaatctccacacacagcttgtggatatgtctatgacatatggacagcagtttggtccaacatacttggatggtgctgatgttacaaaaattaaacctcatgtaaatcatgagggtaagactttctttgtactacctagtgatgacacactacgtagtgaagctttcgagtactaccatactcttgatgagagttttcttggtaggtacatgtctgctttaaaccacacaaagaaatggaaatttcctcaagttggtggtttaacttcaattaaatgggctgataacaattgttatttgtctagtgttttattagcacttcaacagcttgaagtcaaattcaatgcaccagcacttcaagaggcttattatagagcccgtgctggtgatgctgctaacttttgtgcactcatactcgcttacagtaataaaactgttggcgagcttggtgatgtcagagaaactatgacccatcttctacagcatgctaatttggaatctgcaaagcgagttcttaatgtggtgtgtaaacattgtggtcagaaaactactaccttaacgggtgtagaagctgtgatgtatatgggtactctatcttatgataatcttaagacaggtgtttccattccatgtgtgtgtggtcgtgatgctacacaatatctagtacaacaagagtcttcttttgttatgatgtctgcaccacctgctgagtataaattacagcaaggtacattcttatgtgcgaatgagtacactggtaactatcagtgtggtcattacactcatataactgctaaggagaccctctatcgtattgacggagctcaccttacaaagatgtcagagtacaaaggaccagtgactgatgttttctacaaggaaacatcttacactacaaccatcaagcctgtgtcgtataaactcgatggagttacttacacagagattgaaccaaaattggatgggtattataaaaaggataatgcttactatacagagcagcctatagaccttgtaccaactcaaccattaccaaatgcgagttttgataatttcaaactcacatgttctaacacaaaatttgctgatgatttaaatcaaatgacaggcttcacaaagccagcttcacgagagctatctgtcacattcttcccagacttgaatggcgatgtagtggctattgactatagacactattcagcgagtttcaagaaaggtgctaaattactgcataagccaattgtttggcacattaaccaggctacaaccaagacaacgttcaaaccaaacacttggtgtttacgttgtctttggagtacaaagccagtagatacttcaaattcatttgaagttctggcagtagaagacacacaaggaatggacaatcttgcttgtgaaagtcaacaacccacctctgaagaagtagtggaaaatcctaccatacagaaggaagtcatagagtgtgacgtgaaaactaccgaagttgtaggcaatgtcatacttaaaccatcagatgaaggtgttaaagtaacacaagagttaggtcatgaggatcttatggctgcttatgtggaaaacacaagcattaccattaagaaacctaatgagctttcactagccttaggtttaaaaacaattgccactcatggtattgctgcaattaatagtgttccttggagtaaaattttggcttatgtcaaaccattcttaggacaagcagcaattacaacatcaaattgcgctaagagattagcacaacgtgtgtttaacaattatatgccttatgtgtttacattattgttccaattgtgtacttttactaaaagtaccaattctagaattagagcttcactacctacaactattgctaaaaatagtgttaagagtgttgctaaattatgtttggatgccggcattaattatgtgaagtcacccaaattttctaaattgttcacaatcgctatgtggctattgttgttaagtatttgcttaggttctctaatctgtgtaactgctgcttttggtgtactcttatctaattttggtgctccttcttattgtaatggcgttagagaattgtatcttaattcgtctaacgttactactatggatttctgtgaaggttcttttccttgcagcatttgtttaagtggattagactcccttgattcttatccagctcttgaaaccattcaggtgacgatttcatcgtacaagctagacttgacaattttaggtctggccgctgagtgggttttggcatatatgttgttcacaaaattcttttatttattaggtctttcagctataatgcaggtgttctttggctattttgctagtcatttcatcagcaattcttggctcatgtggtttatcattagtattgtacaaatggcacccgtttctgcaatggttaggatgtacatcttctttgcttctttctactacatatggaagagctatgttcatatcatggatggttgcacctcttcgacttgcatgatgtgctataagcgcaatcgtgccacacgcgttgagtgtacaactattgttaatggcatgaagagatctttctatgtctatgcaaatggaggccgtggcttctgcaagactcacaattggaattgtctcaattgtgacacattttgcactggtagtacattcattagtgatgaagttgctcgtgatttgtcactccagtttaaaagaccaatcaaccctactgaccagtcatcgtatattgttgatagtgttgctgtgaaaaatggcgcgcttcacctctactttgacaaggctggtcaaaagacctatgagagacatcctctctcccattttgtcaatttagacaatttgagagctaacaacactaaaggttcactgcctattaatgtcatagtttttgatggcaagtccaaatgcgacgagtctgcttctaagtctgcttctgtgtactacagtcagctgatgtgccaacctattctgttgcttgaccaagctcttgtatcagacgttggagatagtactgaagtttccgttaagatgtttgatgcttatgtcgacaccttttcagcaacttttagtgttcctatggaaaaacttaaggcacttgttgctacagctcacagcgagttagcaaagggtgtagctttagatggtgtcctttctacattcgtgtcagctgcccgacaaggtgttgttgataccgatgttgacacaaaggatgttattgaatgtctcaaactttcacatcactctgacttagaagtgacaggtgacagttgtaacaatttcatgctcacctataataaggttgaaaacatgacgcccagagatcttggcgcatgtattgactgtaatgcaaggcatatcaatgcccaagtagcaaaaagtcacaatgtttcactcatctggaatgtaaaagactacatgtctttatctgaacagctgcgtaaacaaattcgtagtgctgccaagaagaacaacataccttttagactaacttgtgctacaactagacaggttgtcaatgtcataactactaaaatctcactcaagggtggtaagattgttagtacttgttttaaacttatgcttaaggccacattattgtgcgttcttgctgcattggtttgttatatcgttatgccagtacatacattgtcaatccatgatggttacacaaatgaaatcattggttacaaagccattcaggatggtgtcactcgtgacatcatttctactgatgattgttttgcaaataaacatgctggttttgacgcatggtttagccagcgtggtggttcatacaaaaatgacaaaagctgccctgtagtagctgctatcattacaagagagattggtttcatagtgcctggcttaccgggtactgtgctgagagcaatcaatggtgacttcttgcattttctacctcgtgtttttagtgctgttggcaacatttgctacacaccttccaaactcattgagtatagtgattttgctacctctgcttgcgttcttgctgctgagtgtacaatttttaaggatgctatgggcaaacctgtgccatattgttatgacactaatttgctagagggttctatttcttatagtgagcttcgtccagacactcgttatgtgcttatggatggttccatcatacagtttcctaacacttacctggagggttctgttagagtagtaacaacttttgatgctgagtactgtagacatggtacatgcgaaaggtcagaagtaggtatttgcctatctaccagtggtagatgggttcttaataatgagcattacagagctctatcaggagttttctgtggtgttgatgcgatgaatctcatagctaacatctttactcctcttgtgcaacctgtgggtgctttagatgtgtctgcttcagtagtggctggtggtattattgccatattggtgacttgtgctgcctactactttatgaaattcagacgtgcttttggtgagtacaaccatgttgttgctgctaatgcacttttgtttttgatgtctttcactatactctgtctggcaccagcttacagctttctgccgggagtctactcagtcttttacttgtacttgacattctatttcaccaatgatgtttcattcttggctcaccttcaatggtttgccatgttttctcctattgtgcctttttggataacagcaatctatgtattctgtatttctctgaagcactgccattggttctttaacaactatcttaggaaaagagtcatgtttaatggagttacatttagtaccttcgaggaggctgctttgtgtacctttttgctcaacaaggaaatgtacctaaaattgcgtagcgagacactgttgccacttacacagtataacaggtatcttgctctatataacaagtacaagtatttcagtggagccttagatactaccagctatcgtgaagcagcttgctgccacttagcaaaggctctaaatgactttagcaactcaggtgctgatgttctctaccaaccaccacagacatcaatcacttctgctgttctgcagagtggttttaggaaaatggcattcccgtcaggcaaagttgaagggtgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcagttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataacattaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaaggtaagttcaagaaaattgttaagggcactcatcattggatgcttttaactttcttgacatcactattgattcttgttcaaagtacacagtggtcactgtttttctttgtttacgagaatgctttcttgccatttactcttggtattatggcaattgctgcatgtgctatgctgcttgttaagcataagcacgcattcttgtgcttgtttctgttaccttctcttgcaacagttgcttactttaatatggtctacatgcctgctagctgggtgatgcgtatcatgacatggcttgaattggctgacactagcttgtctggttataggcttaaggattgtgttatgtatgcttcagctttagttttgcttattctcatgacagctcgcactgtttatgatgatgctgctagacgtgtttggacactgatgaatgtcattacacttgtttacaaagtctactatggtaatgctttagatcaagctatttccatgtgggccttagttatttctgtaacctctaactattctggtgtcgttacgactatcatgtttttagctagagctatagtgtttgtgtgtgttgagtattacccattgttatttattactggcaacaccttacagtgtatcatgcttgtttattgtttcttaggctattgttgctgctgctactttggccttttctgtttactcaaccgttacttcaggcttactcttggtgtttatgactacttggtctctacacaagaatttaggtatatgaactcccaggggcttttgcctcctaagagtagtattgatgctttcaagcttaacattaagttgttgggtattggaggtaaaccatgtatcaaggttgctactgtacagtctaaaatgtctgacgtaaagtgcacatctgtggtactgctctcggttcttcaacaacttagagtagagtcatcttctaaattgtgggcacaatgtgtacaactccacaatgatattcttcttgcaaaagacacaactgaagctttcgagaagatggtttctcttttgtctgttttgctatccatgcagggtgctgtagacattaataggttgtgcgaggaaatgctcgataaccgtgctactcttcaggctattgcttcagaatttagttctttaccatcatatgccgcttatgccactgcccaggaggcctatgagcaggctgtagctaatggtgattctgaagtcgttctcaaaaagttaaagaaatctttgaatgtggctaaatctgagtttgaccgtgatgctgccatgcaacgcaagttggaaaagatggcagatcaggctatgacccaaatgtacaaacaggcaagatctgaggacaagagggcaaaagtaactagtgctatgcaaacaatgctcttcactatgcttaggaagcttgataatgatgcacttaacaacattatcaacaatgcgcgtgatggttgtgttccactcaacatcataccattgactacagcagccaaactcatggttgttgtccctgattatggtacctacaagaacacttgtgatggtaacacctttacatatgcatctgcactctgggaaatccagcaagttgttgatgcggatagcaagattgttcaacttagtgaaattaacatggacaattcaccaaatttggcttggcctcttattgttacagctctaagagccaactcagctgttaaactacagaataatgaactgagtccagtagcactacgacagatgtcctgtgcggctggtaccacacaaacagcttgtactgatgacaatgcacttgcctactataacaattcgaagggaggtaggtttgtgctggcattactatcagaccaccaagatctcaaatgggctagattccctaagagtgatggtacaggtacaatttacacagaactggaaccaccttgtaggtttgttacagacacaccaaaagggcctaaagtgaaatacttgtacttcatcaaaggcttaaacaacctaaatagaggtatggtgctgggcagtttagctgctacagtacgtcttcaggctggaaatgctacagaagtacctgccaattcaactgtgctttccttctgtgcttttgcagtagaccctgctaaagcatataaggattacctagcaagtggaggacaaccaatcaccaactgtgtgaagatgttgtgtacacacactggtacaggacaggcaattactgtaacaccagaagctaacatggaccaagagtcctttggtggtgcttcatgttgtctgtattgtagatgccacattgaccatccaaatcctaaaggattctgtgacttgaaaggtaagtacgtccaaatacctaccacttgtgctaatgacccagtgggttttacacttagaaacacagtctgtaccgtctgcggaatgtggaaaggttatggctgtagttgtgaccaactccgcgaacccttgatgcagtctgcggatgcatcaacgtttttaaacgggtttgcggtgtaagtgcagcccgtcttacaccgtgcggcacaggcactagtactgatgtcgtctacagggcttttgatatttacaacgaaaaagttgctggttttgcaaagttcctaaaaactaattgctgtcgcttccaggagaaggatgaggaaggcaatttattagactcttactttgtagttaagaggcatactatgtctaactaccaacatgaagagactatttataacttggttaaagattgtccagcggttgctgtccatgactttttcaagtttagagtagatggtgacatggtaccacatatatcacgtcagcgtctaactaaatacacaatggctgatttagtctatgctctacgtcattttgatgagggtaattgtgatacattaaaagaaatactcgtcacatacaattgctgtgatgatgattatttcaataagaaggattggtatgacttcgtagagaatcctgacatcttacgcgtatatgctaacttaggtgagcgtgtacgccaatcattattaaagactgtacaattctgcgatgctatgcgtgatgcaggcattgtaggcgtactgacattagataatcaggatcttaatgggaactggtacgatttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatttgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacctgccttacccagatccatcaagaatattaggcgcaggctgttttgtcgatgatattgtcaaaacagatggtacacttatgattgaaaggttcgtgtcactggctattgatgcttacccacttacaaaacatcctaatcaggagtatgctgatgtctttcacttgtatttacaatacattagaaagttacatgatgagcttactggccacatgttggacatgtattccgtaatgctaactaatgataacacctcacggtactgggaacctgagttttatgaggctatgtacacaccacatacagtcttgcaggctgtaggtgcttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgccactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgccacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcattaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggagaagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgtttcaatgtggctatcacaagggcaaaaattggcattttgtgcataatgtctgatagagatctttatgacaaactgcaatttacaagtctagaaataccacgtcgcaatgtggctacattacaagcagaaaatgtaactggactttttaaggactgtagtaagatcattactggtcttcatcctacacaggcacctacacacctcagcgttgatataaagttcaagactgaaggattatgtgttgacataccaggcataccaaaggacatgacctaccgtagactcatctctatgatgggtttcaaaatgaattaccaagtcaatggttaccctaatatgtttatcacccgcgaagaagctattcgtcacgttcgtgcgtggattggctttgatgtagagggctgtcatgcaactagagatgctgtgggtactaacctacctctccagctaggattttctacaggtgttaacttagtagctgtaccgactggttatgttgacactgaaaataacacagaattcaccagagttaatgcaaaacctccaccaggtgaccagtttaaacatcttataccactcatgtataaaggcttgccctggaatgtagtgcgtattaagatagtacaaatgctcagtgatacactgaaaggattgtcagacagagtcgtgttcgtcctttgggcgcatggctttgagcttacatcaatgaagtactttgtcaagattggacctgaaagaacgtgttgtctgtgtgacaaacgtgcaacttgcttttctacttcatcagatacttatgcctgctggaatcattctgtgggttttgactatgtctataacccatttatgattgatgttcagcagtggggctttacgggtaaccttcagagtaaccatgaccaacattgccaggtacatggaaatgcacatgtggctagttgtgatgctatcatgactagatgtttagcagtccatgagtgctttgttaagcgcgttgattggtctgttgaataccctattataggagatgaactgagggttaattctgcttgcagaaaagtacaacacatggttgtgaagtctgcattgcttgctgataagtttccagttcttcatgacattggaaatccaaaggctatcaagtgtgtgcctcaggctgaagtagaatggaagttctacgatgctcagccatgtagtgacaaagcttacaaaatagaggagctcttctattcttatgctacacatcacgataaattcactgatggtgtttgtttgttttggaattgtaacgttgatcgttacccagccaatgcaattgtgtgtaggtttgacacaagagtcttgtcaaacttgaacttaccaggctgtgatggtggtagtttgtatgtgaataagcatgcattccacactccagctttcgataaaagtgcatttactaatttaaagcaattgcctttcttttactattctgatagtccttgtgagtctcatggcaaacaagtagtgtcggatattgattatgttccactcaaatctgctacgtgtattacacgatgcaatttaggtggtgctgtttgcagacaccatgcaaatgagtaccgacagtacttggatgcatataatatgatgatttctgctggatttagcctatggatttacaaacaatttgatacttataacctgtggaatacatttaccaggttacagagtttagaaaatgtggcttataatgttgttaataaaggacactttgatggacacgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttcagtcatggacaacttggcggtcttcatttaatgataggcttagccaagcgctcacaagattcaccacttaaattagaggattttatccctatggacagcacagtgaaaaattacttcataacagatgcgcaaacaggttcatcaaaatgtgtgtgttctgtgattgatcttttacttgatgactttgtcgagataataaagtcacaagatttgtcagtgatttcaaaagtggtcaaggttacaattgactatgctgaaatttcattcatgctttggtgtaaggatggacatgttgaaaccttctacccaaaactacaagcaagtcaagcgtggcaaccaggtgttgcgatgcctaacttgtacaagatgcaaagaatgcttcttgaaaagtgtgaccttcagaattatggtgaaaatgctgttataccaaaaggaataatgatgaatgtcgcaaagtatactcaactgtgtcaatacttaaatacacttactttagctgtaccctacaacatgagagttattcactttggtgctggctctgataaaggagttgcaccaggtacagctgtgctcagacaatggttgccaactggcacactacttgtcgattcagatcttaatgacttcgtctccgacgcagattctactttaattggagactgtgcaacagtacatacggctaataaatgggaccttattattagcgatatgtatgaccctaggaccaaacatgtgacaaaagagaatgactctaaagaagggtttttcacttatctgtgtggatttataaagcaaaaactagccctgggtggttctatagctgtaaagataacagagcattcttggaatgctgacctttacaagcttatgggccatttctcatggtggacagcttttgttacaaatgtaaatgcatcatcatcggaagcatttttaattggggctaactatcttggcaagccgaaggaacaaattgatggctataccatgcatgctaactacattttctggaggaacacaaatcctatccagttgtcttcctattcactctttgacatgagcaaatttcctcttaaattaagaggaactgctgtaatgtctcttaaggagaatcaaatcaatgatatgatttattctcttctggaaaaaggtaggcttatcattagagaaaacaacagagttgtggtttcaagtgatattcttgttaacaactaaacgaacatgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttgacaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacacttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataaacgaacttatggatttgtttatgagattttttactcttggatcaattactgcacagccagtaaaaattgacaatgcttctcctgcaagtactgttcatgctacagcaacgataccgctacaagcctcactccctttcggatggcttgttattggcgttgcatttcttgctgtttttcagagcgctaccaaaataattgcgctcaataaaagatggcagctagccctttataagggcttccagttcatttgcaatttactgctgctatttgttaccatctattcacatcttttgcttgtcgctgcaggtatggaggcgcaatttttgtacctctatgccttgatatattttctacaatgcatcaacgcatgtagaattattatgagatgttggctttgttggaagtgcaaatccaagaacccattactttatgatgccaactactttgtttgctggcacacacataactatgactactgtataccatataacagtgtcacagatacaattgtcgttactgaaggtgacggcatttcaacaccaaaactcaaagaagactaccaaattggtggttattctgaggataggcactcaggtgttaaagactatgtcgttgtacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgctaatccagcaatggatccaatttatgatgagccgacgacgactactagcgtgcctttgtaagcacaagaaagtgagtacgaacttatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacactccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaagtgacaacagatgtttcatcttgttgacttccaggttacaatagcagagatattgattatcattatgaggactttcaggattgctatttggaatcttgacgttataataagttcaatagtgagacaattatttaagcctctaactaagaagaattattcggagttagatgatgaagaacctatggagttagattatccataaaacgaacatgaaaattattctcttcctgacattgattgtatttacatcttgcgagctatatcactatcaggagtgtgttagaggtacgactgtactactaaaagaaccttgcccatcaggaacatacgagggcaattcaccatttcaccctcttgctgacaataaatttgcactaacttgcactagcacacactttgcttttgcttgtgctgacggtactcgacatacctatcagctgcgtgcaagatcagtttcaccaaaacttttcatcagacaagaggaggttcaacaagagctctactcgccactttttctcattgttgctgctctagtatttttaatactttgcttcaccattaagagaaagacagaatgaatgagctcactttaattgacttctatttgtgctttttagcctttctgctattccttgttttaataatgcttattatattttggttttcactcgaaatccaggatctagaagaaccttgtaccaaagtctaaacgaacatgaaacttctcattgttttgacttgtatttctctatgcagttgcatacgcactgtagtacagcgctgtgcatctaataaacctcatgtgcttgaagatccttgtaaggtacaacactaggggtaatacttatagcactgcttggctttgtgctctaggaaaggttttaccttttcatagatggcacactatggttcaaacatgcacacctaatgttactatcaactgtcaagatccagctggtggtgcgcttatagctaggtgttggtaccttcatgaaggtcaccaaactgctgcatttagagacgtacttgttgttttaaataaacgaacaaattaaaatgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataaacactcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgacaaaaaaaaaaaaaaaaaaaaaaaa

SEQ ID NO: 18 - DNA, 3765 bp, SARS Coronavirus
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagctcgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacaca

SEQ ID NO: 19 - DNA, 663 bp, SARS Coronavirus
atggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacaccccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacag

SEQ ID NO: 20 - DNA, 228 bp, SARS Coronavirus
atgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtc

SEQ ID NO: 21 - DNA, 743 bp, Mahoney Strain Poliovirus Type I
ttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttggttagcactcaaccccagagtgtagcttaggctgatgagtctggacatccctcaccggtgacggtgttccaggctgcgttggcggcctacctatggctaacgcatgggacgctagttgtgaacaaggtgtgaagagcctattgagctacataagaatcctccggcccctgaatgcggctaatcccaacctcggagcaggtggttcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatg

SEQ ID NO: 22 - DNA, 397 bp, Rous Sarcoma Virus
cgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctcc

SEQ ID NO: 23 - DNA, 228 bp, Simian Virus; identical to SEQ ID NO: 20

SEQ ID NO: 24 - DNA, 32 bp, Artificial Sequence
  Synthetic oligonucleotide primer sequence
aataactgcagcgatgtacgggccagatatac

SEQ ID NO: 25 - DNA, 33 bp, Artificial Sequence
  Synthetic oligonucleotide primer sequence
aataagcggccgcggaggtgcacaccaatgtgg

SEQ ID NO: 26 - DNA, 2999 bp, Artificial Sequence
  Plasmid pVAX1
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 27 - DNA, 33 bp, Artificial Sequence
  Synthetic oligonucleotide primer sequence
ttattaagcttatgtactcattcgtttcggaag

SEQ ID NO: 28 - DNA, 31 bp, Artificial Sequence
  Synthetic oligonucleotide primer sequence
tattggtaccgaccagaagatcaggaactcc

SEQ ID NO: 29 - DNA, 52 bp, Artificial Sequence
  Synthetic oligonucleotide primer sequence
taatatctagagccgccgccatgtttattttcttattatttcttactctcac

SEQ ID NO: 30 - DNA, 41 bp, Artificial Sequence
  Synthetic oligonucleotide primer sequence
taatagtttaaacttatcatgtgtaatgtaatttgacaccc

SEQ ID NO: 31 - DNA, 13 bp, Artificial Sequence
  KOZAK sequence
gccgccnccatgg

SEQ ID NO: 32 - DNA, 43 bp, Artificial Sequence
  Synthetic oligonucleotide primer sequence
taatagctagcgccgccgccatggcagacaacggtactattac

SEQ ID NO: 33 - DNA, 42 bp, Artificial Sequence
  Synthetic oligonucleotide primer sequence
gagctgttttaatcattactgtactagcaaagcaatattgtc

SEQ ID NO: 34 - DNA, 33 bp, Artificial Sequence
  Synthetic oligonucleotide primer sequence
gctagtacagttaaaacagctctggggttgtac

SEQ ID NO: 35 - DNA, 34 bp, Artificial Sequence
  Synthetic oligonucleotide primer sequence
cgaatgagtacattatgatacaattgtctgattg

SEQ ID NO: 36 - DNA, 32 bp, Artificial Sequence
  Synthetic oligonucleotide primer sequence
ttgtatcataatgtactcattcgtttcggaag

SEQ ID NO: 37 - DNA, 38 bp, Artificial Sequence
  Synthetic oligonucleotide primer sequence
tattacttaagttatcagaccagaagatcaggaactcc

SEQ ID NO: 38 - DNA, 43 bp, Artificial Sequence; identical to SEQ ID NO: 32
  Synthetic oligonucleotide primer sequence

SEQ ID NO: 39 - DNA, 40 bp, Artificial Sequence
  Synthetic oligonucleotide primer sequence
tattagtttaaacttatcagaccagaagatcaggaactcc

SEQ ID NO: 40 - DNA, 43 bp, Artificial Sequence; identical to SEQ ID NO: 32
  Synthetic oligonucleotide primer sequence

SEQ ID NO: 41 - DNA, 43 bp, Artificial Sequence
  Synthetic oligonucleotide primer sequence
taatagtttaaactcattactgtactagcaaagcaatattgtc

SEQ ID NO: 42 - DNA, 33 bp, Artificial Sequence
  Synthetic oligonucleotide primer sequence
ttattgctagcatgtactcattcgtttcggaag

SEQ ID NO: 43 - DNA, 40 bp, Artificial Sequence; identical to SEQ ID NO: 39
  Synthetic oligonucleotide primer sequence

SEQ ID NO: 44 - protein, 15 aa, Artificial Sequence
  Synthetically prepared polypeptide sequence
DLDRCTTFDDVQAPN

SEQ ID NO: 45 - protein, 15 aa, Artificial Sequence
  Synthetically prepared polypeptide sequence
DDSEPVLKGVKLHYT

SEQ ID NO: 46 - protein, 15 aa, Artificial Sequence
  Synthetically prepared polypeptide sequence
MADNGTITVEELKQL

SEQ ID NO: 47 - protein, 15 aa, Artificial Sequence
  Synthetically prepared polypeptide sequence
TDHAGSNDNIALLVQ

SEQ ID NO: 48 - protein, 15 aa, Artificial Sequence
  Synthetically prepared polypeptide sequence
MYSFVSEETGTLIVN

SEQ ID NO: 49 - protein, 15 aa, Artificial Sequence
  Synthetically prepared polypeptide sequence
VKNLNSSEGVPDLLV